SEQUENCE LISTING (203 sequences)

SEQ ID NO: 1 - protein, 736 aa, Adeno-associated dependoparvovirus A
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, Adeno-associated dependoparvovirus A [sequence omitted]

SEQ ID NO: 3 - protein, 736 aa, Adeno-associated dependoparvovirus A
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 734 aa, Adeno-associated dependoparvovirus A
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 5 - protein, 724 aa, Adeno-associated dependoparvovirus B
MAFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRSTRPIGTRYLTRPL

SEQ ID NO: 6 - protein, 736 aa, Adeno-associated dependoparvovirus A
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - protein, 737 aa, Adeno-associated dependoparvovirus A
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 8 - protein, 738 aa, Adeno-associated dependoparvovirus A
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 9 - protein, 736 aa, Adeno-associated dependoparvovirus A
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 738 aa, Adeno-associated dependoparvovirus A
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 11 - protein, 733 aa, Adeno-associated dependoparvovirus A
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 12 - protein, 742 aa, Adeno-associated dependoparvovirus A
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 13 - protein, 733 aa, Adeno-associated dependoparvovirus A
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 14 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 15 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 16 - protein, 733 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 17 - protein, 10 aa, synthetic construct
QNQSGSAQNK

SEQ ID NO: 18 - protein, 7 aa, synthetic construct
NQSGSAQ

SEQ ID NO: 19 - protein, 10 aa, synthetic construct
NTPSGTTTQS

SEQ ID NO: 20 - protein, 7 aa, synthetic construct
TPSGTTT

SEQ ID NO: 21 - protein, 10 aa, synthetic construct
QGTTSGTTNQ

SEQ ID NO: 22 - protein, 7 aa, synthetic construct
GTTSGTT

SEQ ID NO: 23 - protein, 10 aa, synthetic construct
TTGTTLNAGT

SEQ ID NO: 24 - protein, 7 aa, synthetic construct
TGTTLNA

SEQ ID NO: 25 - protein, 10 aa, synthetic construct
NNTGGVQFNK

SEQ ID NO: 26 - protein, 7 aa, synthetic construct
NTGGVQF

SEQ ID NO: 27 - protein, 10 aa, synthetic construct
QNQSGSAQNK

SEQ ID NO: 28 - protein, 7 aa, synthetic construct
NQSGSAQ

SEQ ID NO: 29 - protein, 10 aa, synthetic construct
QSNPGGTAGN

SEQ ID NO: 30 - protein, 7 aa, synthetic construct
SNPGGTA

SEQ ID NO: 31 - protein, 10 aa, synthetic construct
QTTGGTANTQ

SEQ ID NO: 32 - protein, 7 aa, synthetic construct
TTGGTAN

SEQ ID NO: 33 - protein, 10 aa, synthetic construct
INGSGQNQQT

SEQ ID NO: 34 - protein, 7 aa, synthetic construct
NGSGQNQ

SEQ ID NO: 35 - protein, 10 aa, synthetic construct
QSTGGTQGTQ

SEQ ID NO: 36 - protein, 7 aa, synthetic construct
STGGTQG

SEQ ID NO: 37 - protein, 10 aa, synthetic construct
TSGETLNQGN

SEQ ID NO: 38 - protein, 7 aa, synthetic construct
SGETLNQ

SEQ ID NO: 39 - protein, 10 aa, synthetic construct
TTGNSLNQGT

SEQ ID NO: 40 - protein, 7 aa, synthetic construct
TGNSLNQ

SEQ ID NO: 41 - protein, 10 aa, synthetic construct
QTASGTQQSR

SEQ ID NO: 42 - protein, 7 aa, synthetic construct
TASGTQQ

SEQ ID NO: 43 - protein, 10 aa, synthetic construct
QTTGTGGTQT

SEQ ID NO: 44 - protein, 7 aa, synthetic construct
TTGTGGT

SEQ ID NO: 45 - protein, 10 aa, synthetic construct
QSTGGTAGTQ

SEQ ID NO: 46 - protein, 7 aa, synthetic construct
STGGTAG

SEQ ID NO: 47 - protein, 10 aa, synthetic construct
TSGETLNQGN

SEQ ID NO: 48 - protein, 7 aa, synthetic construct
SGETLNQ

SEQ ID NO: 49 - protein, 7 aa, synthetic construct
EGGTVHA

SEQ ID NO: 50 - protein, 7 aa, synthetic construct
FYGTDSA

SEQ ID NO: 51 - protein, 7 aa, synthetic construct
HGQSASR

SEQ ID NO: 52 - protein, 7 aa, synthetic construct
DTPTNQA

SEQ ID NO: 53 - protein, 7 aa, synthetic construct
ITRQAYQ

SEQ ID NO: 54 - protein, 7 aa, synthetic construct
RMFKSNQ

SEQ ID NO: 55 - protein, 7 aa, synthetic construct
GVSLGGG

SEQ ID NO: 56 - protein, 7 aa, synthetic construct
KHFLQGE

SEQ ID NO: 57 - protein, 7 aa, synthetic construct
MGRERAG

SEQ ID NO: 58 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIEGGTVHAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 59 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIFYGTDSAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 60 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIHGQSASRQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 61 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIDTPTNQAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 62 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIITRQAYQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 63 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIRMFKSNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 64 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 65 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKHFLQGEQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 66 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIMGRERAGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 67 - protein, 7 aa, synthetic construct
LNSSVPS

SEQ ID NO: 68 - protein, 7 aa, synthetic construct
YMDHQVS

SEQ ID NO: 69 - protein, 7 aa, synthetic construct
TSDSLVS

SEQ ID NO: 70 - protein, 7 aa, synthetic construct
NAVGALS

SEQ ID NO: 71 - protein, 7 aa, synthetic construct
MPISHHE

SEQ ID NO: 72 - protein, 7 aa, synthetic construct
DSGARGA

SEQ ID NO: 73 - protein, 7 aa, synthetic construct
NVALALG

SEQ ID NO: 74 - protein, 7 aa, synthetic construct
GALRMGM

SEQ ID NO: 75 - protein, 7 aa, synthetic construct
LSGEGAV

SEQ ID NO: 76 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLNSSVPSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 77 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQYMDHQVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 78 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTSDSLVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 79 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNAVGALSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 80 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMPISHHETGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 81 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDSGARGATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 82 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVALALGTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 83 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGALRMGMTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 84 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLSGEGAVTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 85 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIEGGTVHAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLNSSVPSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 86 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIEGGTVHAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQYMDHQVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 87 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIEGGTVHAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTSDSLVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 88 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIEGGTVHAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNAVGALSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 89 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIEGGTVHAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMPISHHETGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 90 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIEGGTVHAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDSGARGATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 91 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIEGGTVHAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVALALGTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 92 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIEGGTVHAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGALRMGMTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 93 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIEGGTVHAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLSGEGAVTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 94 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIFYGTDSAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLNSSVPSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 95 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIFYGTDSAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQYMDHQVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 96 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIFYGTDSAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTSDSLVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 97 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIFYGTDSAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNAVGALSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 98 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIFYGTDSAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMPISHHETGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 99 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIFYGTDSAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDSGARGATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 100 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIFYGTDSAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVALALGTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 101 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIFYGTDSAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGALRMGMTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 102 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIFYGTDSAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLSGEGAVTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 103 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIHGQSASRQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLNSSVPSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 104 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIHGQSASRQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQYMDHQVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 105 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIHGQSASRQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTSDSLVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 106 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIHGQSASRQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNAVGALSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 107 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIHGQSASRQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMPISHHETGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 108 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIHGQSASRQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDSGARGATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 109 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIHGQSASRQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVALALGTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 110 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIHGQSASRQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGALRMGMTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 111 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIHGQSASRQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLSGEGAVTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 112 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIDTPTNQAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLNSSVPSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 113 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIDTPTNQAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQYMDHQVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 114 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIDTPTNQAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTSDSLVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 115 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIDTPTNQAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNAVGALSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 116 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIDTPTNQAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMPISHHETGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 117 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIDTPTNQAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDSGARGATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 118 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIDTPTNQAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVALALGTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 119 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIDTPTNQAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGALRMGMTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 120 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIDTPTNQAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLSGEGAVTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 121 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIITRQAYQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLNSSVPSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 122 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIITRQAYQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQYMDHQVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 123 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIITRQAYQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTSDSLVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 124 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIITRQAYQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNAVGALSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 125 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIITRQAYQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMPISHHETGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 126 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIITRQAYQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDSGARGATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 127 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIITRQAYQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVALALGTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 128 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIITRQAYQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGALRMGMTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 129 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIITRQAYQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLSGEGAVTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 130 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIRMFKSNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLNSSVPSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 131 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIRMFKSNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQYMDHQVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 132 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIRMFKSNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTSDSLVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 133 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIRMFKSNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNAVGALSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 134 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIRMFKSNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMPISHHETGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 135 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIRMFKSNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDSGARGATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 136 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIRMFKSNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVALALGTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 137 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIRMFKSNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGALRMGMTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 138 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIRMFKSNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLSGEGAVTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 139 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLNSSVPSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 140 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQYMDHQVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 141 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTSDSLVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 142 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNAVGALSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 143 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMPISHHETGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 144 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDSGARGATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 145 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVALALGTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 146 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGALRMGMTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 147 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLSGEGAVTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 148 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKHFLQGEQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLNSSVPSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 149 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKHFLQGEQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQYMDHQVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 150 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKHFLQGEQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTSDSLVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 151 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKHFLQGEQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNAVGALSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 152 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKHFLQGEQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMPISHHETGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 153 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKHFLQGEQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDSGARGATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 154 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKHFLQGEQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVALALGTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 155 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKHFLQGEQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGALRMGMTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 156 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKHFLQGEQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLSGEGAVTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 157 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIMGRERAGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLNSSVPSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 158 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIMGRERAGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQYMDHQVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 159 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIMGRERAGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTSDSLVSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 160 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIMGRERAGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNAVGALSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 161 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIMGRERAGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMPISHHETGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 162 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIMGRERAGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDSGARGATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 163 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIMGRERAGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVALALGTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 164 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIMGRERAGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGALRMGMTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 165 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIMGRERAGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLSGEGAVTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 166 - protein, 737 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTSNGRGVTLGFSQGGPNTMANQAKNWLPGPCYRQQRVSTYPLQNNNSNFAWTAGTKYHLNGRNSLANPGIAMATHKDDEERFFPSNGILIFGKQNAARDNADYSDVMLTSEEEIKTTNPVATEEYGIVADNGQTQTTAPQIGTVNSQGALPGMVWQNRDVYLQGPIWAKIPHTDGNFHPSPLMGGFGLKHPPPQILIKNTPVPADPRSTFNGDKLNSFITQYSTGQVSVEIEWELQKENSKRWNPEIQYTSNYYKSTSVDFAVNTEGVYSEPRPIGTRYLTRNL

SEQ ID NO: 167 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 168 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSSERRDLLFSRGSPAGMSVQPKNWLPGPCYRQQRVSKTPGGNATRNFTWTGASKYNLNGRESIINPGTAMASHKDDEDKFFPMSGVMIFGKESAGASNTALDNVMITDEEEIKATNPVATERFGTVAVNFQSSDLDPKATEVEAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 169 - protein, 737 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 170 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSVAGPSNMAVQGRNWLPGPCYRQQRVSKTSADNNNSEFAWTGATKYHLNGRDSLVNPGPAMASHKDDEEKFFPQSGVLIFGKQGSEKTNVDIEKVMITDEEEIRTTNPVATEQYGSVSTNLQQQNTAPATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 171 - protein, 735 aa, synthetic construct
MAADGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYEIPATGSDSLIEYETHSTLDGRWSALTPGPPMATAGPADSKFSNSQLIFAGPKQNGNTATVPGTLIFTSEEELAATNATDTDMWGNLPGGDQSSSNLPPVMGLGALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 172 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQXXXXXXXNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 173 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNXXXXXXXQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 174 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQXXXXXXXNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 175 - protein, 734 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTXXXXXXXGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 176 - protein, 724 aa, synthetic construct
MAFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNXXXXXXXNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRSTRPIGTRYLTRPL

SEQ ID NO: 177 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQXXXXXXXNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 178 - protein, 737 aa, synthetic construct
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQXXXXXXXGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 179 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQXXXXXXXTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 180 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIXXXXXXXQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 181 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQXXXXXXXTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 182 - protein, 733 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTXXXXXXXGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 183 - protein, 742 aa, synthetic construct
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTXXXXXXXGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 184 - protein, 733 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQXXXXXXXSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 185 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQXXXXXXXQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 186 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQXXXXXXXTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 187 - protein, 733 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTXXXXXXXGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 188 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTXXXXXXXXXXDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 189 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTXXXXXXXXXXRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 190 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTXXXXXXXXXXSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 191 - protein, 734 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTXXXXXXXXXXATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 192 - protein, 724 aa, synthetic construct
MAFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTXXXXXXXXXXNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRSTRPIGTRYLTRPL

SEQ ID NO: 193 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTXXXXXXXXXXDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 194 - protein, 737 aa, synthetic construct
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTXXXXXXXXXXRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 195 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTXXXXXXXXXXTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 196 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTXXXXXXXXXXLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 197 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTXXXXXXXXXXQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 198 - protein, 733 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTXXXXXXXXXXAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 199 - protein, 742 aa, synthetic construct
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTXXXXXXXXXXATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 200 - protein, 733 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTXXXXXXXXXXLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 201 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTXXXXXXXXXXLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 202 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTXXXXXXXXXXQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 203 - protein, 733 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTXXXXXXXXXXAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

(19) United States
(12) Patent Application Publication
(10) Pub. No.: US 2026/0234659 A1
(43) Pub. Date: Aug. 13, 2026

(54) COMPOSITIONS COMPRISING AAVS AND METHODS FOR TREATING GASTROINTESTINAL DISEASES

(71) Applicant: Duke University, Durham, NC (US)

(72) Inventors: Jatin Roper, Durham, NC (US); Aravind Asokan, Durham, NC (US); Lawrence Patrick Havlik, Durham, NC (US)

(21) Appl. No.: 19/154,135

(22) PCT Filed: Feb. 7, 2024
(86) PCT No.: PCT/US2024/014888

§ 371 Date: Aug. 6, 2025

Related U.S. Application Data
(60) Provisional application No. 63/443,742, filed on Feb. 7, 2023.

(51) Int. Cl. C12N 15/86; A61K 48/00; C07K 14/005; C12N 9/22; C12N 15/11

(52) U.S. Cl. CPC C12N 15/86; A61K 48/0041; A61K 48/005; C07K 14/005; C12N 9/226; C12N 15/111; C12N 2310/20; C12N 2750/14122; C12N 2750/14143; C12N 2750/14145

(57) ABSTRACT

Recombinant adeno-associated virus (rAAV) vectors are leading gene delivery platforms, and several rAAV-mediated therapies have recently been approved for clinical use. Disclosed herein are compositions comprising variant AAV capsid proteins demonstrating improved tropism and improved transduction efficiency for intestinal epithelium and colonic epithelium, and methods of using vectors comprising these variant AAV capsid proteins to efficiently deliver a payload to target cells or tissues and to treat a subject in need thereof.

### I. CROSS-REFERENCE TO RELATED APPLICATIONS

This application claims the benefit of priority to U.S. Provisional Patent Application No. 63/443,742 filed 7 Feb. 2023, which is incorporated by reference herein in its entirety.

### II. REFERENCE TO THE SEQUENCE LISTING

The Sequence Listing submitted 7 Feb. 2024 as an XML file named “23-2072-WO Sequence Listing”, created on 6 Feb. 2024 and having a size of 285 kilobytes is hereby incorporated by reference pursuant to 37 C.F.R. § 1.52(e)(5).

### III. BACKGROUND OF THE INVENTION

Cystic fibrosis is the result of a defect in the cystic fibrosis transmembrane regulator (CFTR), which is responsible for the excretion of salt. The defect results in viscous secretions in multiple organ systems. Improvements in nutrition have improved the average life expectancy of patients with cystic fibrosis. However, there remains an urgent need to restore the functionality of CFTR in these patients.

### IV. BRIEF SUMMARY OF THE INVENTION

Disclosed herein is a variant AAV capsid protein. Disclosed herein is a variant AAV capsid protein having one or more substitutions in variable region IV (VR-IV). Disclosed herein is a variant AAV capsid protein having one or more substitutions in variable region VIII (VR-VIII). Disclosed herein is a variant AAV capsid protein having one or more substitutions in variable region IV and/or variable region VIII. Disclosed herein is an adeno-associated virus (AAV) capsid protein comprising the sequence set forth in SEQ ID NO:172 or SEQ ID NO:188. Disclosed herein is an adeno-associated virus (AAV) capsid protein comprising the sequence set forth in SEQ ID NO:173 or SEQ ID NO:189. Disclosed herein is an adeno-associated virus (AAV) capsid protein comprising the sequence set forth in SEQ ID NO:174 or SEQ ID NO:190. Disclosed herein is an adeno-associated virus (AAV) capsid protein comprising the sequence set forth in SEQ ID NO:175 or SEQ ID NO:191. Disclosed herein is an adeno-associated virus (AAV) capsid protein comprising the sequence set forth in SEQ ID NO:176 or SEQ ID NO:192. Disclosed herein is an adeno-associated virus (AAV) capsid protein comprising the sequence set forth in SEQ ID NO: 177 or SEQ ID NO:193. Disclosed herein is an adeno-associated virus (AAV) capsid protein comprising the sequence set forth in SEQ ID NO:178 or SEQ ID NO:194. Disclosed herein is an adeno-associated virus (AAV) capsid protein comprising the sequence set forth in SEQ ID NO:179 or SEQ ID NO:195. Disclosed herein is an adeno-associated virus (AAV) capsid protein comprising the sequence set forth in SEQ ID NO: 180 or SEQ ID NO: 196. Disclosed herein is an adeno-associated virus (AAV) capsid protein comprising the sequence set forth in SEQ ID NO:181 or SEQ ID NO:197. Disclosed herein is an adeno-associated virus (AAV) capsid protein comprising the sequence set forth in SEQ ID NO:182 or SEQ ID NO:198. Disclosed herein is an adeno-associated virus (AAV) capsid protein comprising the sequence set forth in SEQ ID NO: 183 or SEQ ID NO: 199. Disclosed herein is an adeno-associated virus (AAV) capsid protein comprising the sequence set forth in SEQ ID NO:184 or SEQ ID NO:200. Disclosed herein is an adeno-associated virus (AAV) capsid protein comprising the sequence set forth in SEQ ID NO:185 or SEQ ID NO:201. Disclosed herein is an adeno-associated virus (AAV) capsid protein comprising the sequence set forth in SEQ ID NO: 186 or SEQ ID NO:202. Disclosed herein is an adeno-associated virus (AAV) capsid protein comprising the sequence set forth in SEQ ID NO: 187 or SEQ ID NO:203.

Disclosed herein is a method for delivering a payload, the method comprising contacting one or more cells in a subject with a therapeutically effective amount of a disclosed nucleic acid molecule encoding a gene of interest or a transgene; and expressing the encoded gene of interest or transgene. Disclosed herein is a method for delivering a payload, the method comprising contacting one or more cells in a subject with a therapeutically effective amount of an AAV vector encoding a gene of interest or a transgene; and expressing the encoded gene of interest or transgene. Disclosed herein is a method for delivering a payload, the method comprising contacting one or more cells in a subject with a therapeutically effective amount of a disclosed AAV vector (i) comprising a disclosed variant capsid protein and (ii) encoding a gene of interest or a transgene; and expressing the encoded gene of interest or transgene. Disclosed herein is a method of delivering a payload, the method comprising contacting one or more cells with a disclosed nucleic acid molecule or with a disclosed rAAV vector, wherein the contacting allows for expression of the payload in the one or more cells.

Disclosed herein is a method of treating a subject, the method comprising administering one or more times to a subject in need thereof a therapeutically effective amount of a disclosed AAV vector or a disclosed pharmaceutical composition. Disclosed herein is a method for treating a subject, the method comprising contacting one or more cells in a subject with a therapeutically effective amount of a disclosed AAV vector encoding a payload, a gene of interest or a transgene; and expressing the encoded payload, the encoded gene of interest, or the encoded transgene. Disclosed herein is a method for treating a subject, the method comprising contacting one or more cells in a subject with a therapeutically effective amount of a disclosed AAV vector (i) comprising a disclosed variant capsid protein and (ii) encoding a payload, a gene of interest, or a transgene; and expressing the encoded payload, the encoded gene of interest, or the encoded transgene. Disclosed herein is a method of treating a subject, the method comprising contacting one or more cells in the subject with a disclosed nucleic acid molecule or with a disclosed rAAV vector, wherein the contacting allows for expression of the encoded payload, the encoded gene of interest, or the encoded transgene in the one or more cells.

### V. BRIEF DESCRIPTION OF THE FIGURES

FIG. 1A-FIG. 1C show somatic gene editing in the intestine in vivo by base editing is predicted to repair the W1282X nonsense mutation in accordance with one embodiment of the present disclosure.

FIG. 1A shows the intestinal epithelium demonstrating the crypt-villi structure (orange boxes indicate the position of intestinal stem cells) (adopted from Gehart H, et al. (2018) Nat Rev Gastroenterol Hepatol. 16:19-34).

FIG. 1B shows the ABE is guided to a specific DNA segment by a guide RNA (SgRNA), with a target base (A₆) within the editing window of the ABE (position 4-7). The ABE results in an A to G conversion (adopted from Yarra R, et al. (2021) Plant Cell Rep. 40:595-604).

FIG. 1C shows the proposed BE approach in the W1282X CF mouse model. The protospacer adjacent motif (PAM) of the sgRNA is in brackets. The most common (73%) edit restores the wild-type tryptophan AA. The second most common (17.4%) produces tryptophan and a likely clinically insignificant R1283G mutation. Together, the expected likelihood to restore the CFTR protein to functionality is 90.4% of all edits produced by the BE strategy. BE predictions from BE HIVE (www.crisprbehive.design).

FIG. 2A-FIG. 2B show images and schematics of colonoscopy-guided injection and evolution scheme of cross-species compatible AAVs (ccAAVs) in accordance with one embodiment of the present disclosure.

FIG. 2A is a schematic of colonoscopy-guided injection demonstrating placement of the needle into the submucosa, resulting in a “bubble” under the mucosa, as well as representative colonoscopic images from mouse, pig, and human, demonstrating the cross-species utility of the experimental approach.

FIG. 2B shows capsid libraries are cycled through mice, pigs and monkeys. The parental capsid library is injected in mice and AAV genomes are amplified from murine tissues, generating anew capsid library, which is then injected into a pig. This process is repeated until a final capsid library is generated from the rhesus macaque tissue harvest. This process generates AAVs with the highest tropism for target tissues in multiple species and greatest potential for clinical translation into humans.

FIG. 3A-FIG. 3C shows data showing that colonoscopy-guided injection of adenoviral GFP efficiently transduces colonic stem cells and AAV.cc47 transduces target tissues with high efficiency compared to AAV9 and AAV9 and AAV.cc47 packaging self-complementary Cbh-mCherry were delivered IV (1e12 vg/mouse) in accordance with one embodiment of the present disclosure.

FIG. 3A shows a representative image of immunohistochemistry labeling of GFP+ cells (brown staining) in murine colonic tissue collected 4 days after colonoscopy-guided injection of adenoviral-GFP. Scale bar is 200 μm. Insets demonstrate GFP+ colonic epithelium. Scale bar is 20 m.

FIG. 3B shows the number of colonic crypts with >2 GFP+ cells were quantified as a percentage of the injected area for adenoviral-GFP and lentiviral-GFP (identified as the cross section with GFP positivity in the muscularis mucosae). n=10 animals; ****p<0.0001.

FIG. 3C shows representative images and total mCherry Fluorescence in the kidney 28 days post injection are shown (insets=white dotted line). *p<0.05.

FIG. 4A-FIG. 4D provide images, graphs, and schematics showing AAVcc.47 results in high efficiency of intestinal epithelial transfection.

FIG. 4A shows a schematic of evaluation of AAVcc.47 carrying either a fluorescent marker, GFP or Cre (for injection into R26R-LSL-tdTomato mice). Mice were injected by intravenous or colonoscopy-guided routes, then sacrificed at various timepoints to evaluate efficiency of viral transfection.

FIG. 4B provides representative IHC images of intestine collected 4 days after injection of AAVcc.47-scCbh-GFP by IV or colonoscopy routes (n=10 per group). Brown-stained cells have expressed the GFP introduced by the virus.

FIG. 4C shows representative IHC images of intestine collected 4 days or one month after injection of either AAVcc.47-CMV-Cre or AAVcc.47-scCbh-Cre by IV or colonoscopy routes. Brown-stained cells have expressed tdTomato after removal of the STOP codon by Cre expression.

FIG. 4D shows preliminary quantification of the % of lineage trace events (indicating stem cell transfection) in the indicated groups. n=1 (IV, AAVcc.47-scCbh-Cre), n=3 (IV, AAVcc.47-CMV-Cre).

FIG. 5A-FIG. 5D are images, schematics and data showing successful electroporation of CFTR^{W1282X/W1282X} mouse embryonic fibroblasts with ABE and sgRNA to edit W1282X mutation.

FIG. 5A shows forskolin stimulation assay was performed on organoids isolated from CFTR^{wt/wt} and CFTR^{W1282X/W1282X} littermates. Organoids were imaged pre and post application of 5 mM forskolin. Scale bar is 20 m.

FIG. 5B shows representative images of CFTR^{W1282X/W1282X} mouse embryonic fibroblasts post electroporation with either pABEmax-GFP or pNG-ABEmax and sgRNA targeting the W1282X CFTR mutation. Scale bar is 50 μm. Electroporation efficiency based on ABEmax-GFP (% of GFP+ cells/total cells) averaged 24% (n=5).

FIG. 5C shows steps for validation of on-target editing using the electroporated MEFs in FIG. 5A.

FIG. 5D shows the percentage of base editing in MEFs. After the Sanger sequencing was performed, the sequences were analyzed using EditR software. Percentage of edits was quantified at each adenosine base within the guide sequence as these are the potential sites of base editing with an ABE. The mutated base, and therefore the position of interest, is located at position 6 (denoted by the dashed box). For NG-ABEmax+CFTR sgRNA, n=2. For the other two conditions, n=1. Samples had a transfection efficiency of approximately 25%.

FIG. 6A-FIG. 6B show that AAV.cc47 efficiently transduced intestinal and colonic epithelium. Cbh-Cre packaged by AAV.cc47 were delivered at a dose of 6e11 vg/mouse into LSL-tdTomato reporter mice.

FIG. 6A shows tdTomato immunohistochemistry in jejunum.

FIG. 6B shows fluorescence in the (D) duodenum, (J) jejunum, (I) ileum, and (C) colon epithelium, which was quantified at one month. Scale bar 20 mm.

sequences were analyzed using EditR software. Percentage of edits was quantified at each adenosine base within the guide sequence as these are the potential sites of base editing with an ABE. The mutated base, and therefore the position of interest, is located at position 6 (denoted by the dashed box). For NG-ABEmax+CFTR sgRNA. n=2. For the other two conditions. n=1. Samples had a transfection efficiency of approximately 25%.

FIG. 7A-FIG. 7B shows that AAV.cc47 mCherry transduced target tissues with high efficiency. AAV9/AAV.cc47 vectors were delivered through intravenous injection at a dose of 7.42e9vg per wild-type B6 mouse. Mice were harvested at the indicated time points, for immunohistochemistry and quantification of the transduction efficiency of AAVcc.47 scCbh-mCherry (FIG. 7A), and AAV9 scCbh-mCherry (FIG. 7B). Intestinal crypts with RFP+ cells were quantified as a percentage of positive crypts, and the native mCherry expression was analyzed using immunofluorescence staining. tdTomato (Red), Blue (DAPI). Pancreas was also analyzed from selected mice.

FIG. 8A-FIG. 8D shows that AAV.cc47-Cre transduced target tissues with high efficiency. AAV.cc47-scCbh-Cre and CMV-Cre vectors were delivered through intravenous injection at a dose of 7.5e9vg per tdTomato (Ai9) fluorescent reporter mouse. Mice were harvested at the indicated time points, for immunohistochemistry and quantification of the transduction efficiency of AAVcc.47 scCbh-Cre and AAVcc.47 CMV-Cre. Intestinal crypts with RFP+ cells were quantified as a percentage of positive crypts, and the native mCherry expression was analyzed using immunofluorescence staining. tdTomato (Red), Blue (DAPI). Pancreas and lung were also analyzed from selected mice.

FIG. 9 shows AAV.cc47-scCbh-GFP transduced the intestinal epithelium in a large animal pig model. AAVcc.47-scCbh-GFP was delivered directly into the superior mesenteric artery via surgical access at a dose of 1e14vg/pig and then sacrificed at day 2 or 3. Analysis of the jejunum showed efficient transduction of the intestinal epithelium and notable GFP positive stem cells.

FIG. 10 shows the alignment of sequences for SEQ ID NO:01-SEQ ID NO: 16. The boxes reflect target area #1 (VR-IV) and target area #2 (VR-VIII). In FIG. 10, AAV1 is SEQ ID NO:01, AAV2 is SEQ ID NO:02, AAV3b is SEQ ID NO:03, AAV4 is SEQ ID NO:04, AAV5 is SEQ ID NO:05. AAV6 is SEQ ID NO:06, AAV7 is SEQ ID NO:07, AAV8 is SEQ ID NO:08, AAV9 is SEQ ID NO:09, AAV10 is SEQ ID NO:10, AAV1l is SEQ ID NO:11, AAV12 is SEQ ID NO:12, AAV13 is SEQ ID NO:13, AAVrh8 is SEQ ID NO:14, AAVrh10 is SEQ ID NO:15, and AAVrh32.33 is SEQ ID NO:16.

### VI. DETAILED DESCRIPTION OF THE INVENTION

The present disclosure describes isolated nucleic acid molecules, viral vectors, viral capsid proteins, viral particles, plasmids, cells, kits, pharmaceutical formulations, and compositions thereof and methods of using the disclosed compositions. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, vectors, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “approximately” or “about.” as applied to one or more values of interest, refers to a value that is similar to a stated reference value. In an aspect, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction of the stated reference value unless otherwise stated or otherwise evident from the context.

As used herein, the term “in vitro” refers to events or experiments that occur in an artificial environment, e.g., in a petri dish, test tube, cell culture, etc., rather than within a multicellular organism. As used herein, the term “in vivo” refers to events or experiments that occur within a multicellular organism.

As used herein, the term “comparable” in the context of a particular value and a reference value means that the particular value is consistent with the reference value, or that the deviation from the reference value (above or below) is at most 10%.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration. In an aspect, a subject can be a human being. The term “subject” includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a disease, a disorder, an infection, a symptom, and/or a complication, be suspected of having a disease, a disease, a disorder, an infection, a symptom, and/or a complication, or be at risk of developing a disease, a disorder, an infection, a symptom, and/or a complication. For example, a subject can have risk factors for developing a disease, a disorder, an infection, a symptom, and/or a complication. A subject can be at risk due to genetic predisposition, employment type (e.g., a health care worker, a miner), attendance at a specific location (e.g., school), attendance at social events (e.g., sporting events, concerns, religious services, political rallies and events, social justice rallies, marches, and events, etc.), by use of public transportation or public services, exposure to natural and man-made disasters (e.g., Chernobyl, 9/11 attacks, etc.).

In an aspect, a subject can have cystic fibrosis. In an aspect, a subject can have any genetic disease or disorder that affects the gastrointestinal system. In an aspect, a subject can have colitis (e.g., infections colitis, ulcerative colitis, Crohn's disease, ischemic colitis, or any combination thereof). In an aspect, a subject can have peptic ulcer disease, gastritis, gastroenteritis, celiac disease, Crohn's disease, gallstones, fecal incontinence, lactose intolerance, Hirschsprung disease, abdominal adhesions, Barrett's esophagus, appendicitis, indigestion (dyspepsia), intestinal pseudo-obstruction, pancreatitis, short bowel syndrome, Whipple's disease, Zollinger-Ellison syndrome, malabsorption syndromes, and hepatitis.

In an aspect, a subject can have a genetic disorder. Genetic disorders include but are not limited to cystic fibrosis, Hurler Syndrome, α-1-antitrypsin (A1AT) deficiency, Parkinson's disease, Alzheimer's disease, albinism, Amyotrophic lateral sclerosis, Asthma, Thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa. Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, and cancer.

In an aspect, a subject can have cancer. Cancer includes, but is not limited to, ovarian cancer, epithelial ovarian cancer, non-Hodgkin's lymphomas (such as diffuse large B-cell lymphoma), acute myeloid leukemia, thymus cancer, brain cancer, lung cancer, squamous cell cancer, skin cancer, eye cancer, retinoblastoma, intraocular melanoma, oral cavity and oropharyngeal cancer, bladder cancer, gastric cancer, stomach cancer, pancreatic cancer, breast cancer, cervical cancer, head and neck cancer, renal cancer, kidney cancer, liver cancer, prostate, colorectal cancer, bone (e.g., metastatic bone), esophageal cancer, testicular cancer, gynecological cancer, thyroid cancer, central nervous system lymphomas, AIDS-related cancers (e.g., lymphoma and Kaposi's sarcoma), viral-induced cancers such as cervical carcinoma (human papillomavirus). B-cell lymphoproliferative disease and nasopharyngeal carcinoma (Epstein-Barr virus), Kaposi's sarcoma and primary effusion lymphomas, hepatocellular carcinoma (hepatitis B and hepatitis C viruses), and T-cell leukemias (human T-cell leukemia virus-1), B cell acute lymphoblastic leukemia, Burkitt's leukemia, juvenile myelomonocytic leukemia, hairy cell leukemia, Hodgkin's disease, multiple myeloma, mast cell leukemia, and mastocytosis.

As used herein, “effective amount” and “amount effective” can refer to an amount that is sufficient to achieve the desired result such as, for example, the treatment and/or prevention of a disease, a disorder, an infection, a symptom, and/or a complication, or a suspected disease, disorder, infection, symptom, and/or complication. In an aspect, an effective amount can alleviate and/or improve one or more symptoms and/or complications associated with cystic fibrosis. In an aspect, a “therapeutically effective amount” refers to an amount (i.e., vector genome/body weight or vg/kg) that is sufficient to achieve the desired therapeutic result or to have an effect on undesired symptoms, but is generally insufficient to cause adverse side effects. In an aspect, “therapeutically effective amount” means an amount of a disclosed composition that (i) treats the particular disease, disorder, and/or infection, (ii) attenuates, ameliorates, or eliminates one or more symptoms of the particular disease, condition, and/or disorder, or (iii) delays the onset of one or more symptoms of the particular disease, condition, and/or disorder described herein. The specific therapeutically effective dose level for any particular patient will depend upon a variety of factors including the disorder being treated and the severity of the disorder; the specific disclosed compositions and/or a pharmaceutical preparation comprising one or more disclosed compositions, or methods employed; the age, body weight, general health, sex and diet of the patient; the time of administration; the route of administration; the rate of excretion of the disclosed compositions and/or a pharmaceutical preparation comprising one or more disclosed compositions employed; the duration of the treatment; drugs used in combination or coincidental with a disclosed compositions and/or a pharmaceutical preparation comprising one or more disclosed compositions employed, and other like factors well known in the medical arts. For example, it is well within the skill of the art to start doses of a disclosed composition and/or a pharmaceutical preparation comprising one or more disclosed composition at levels lower than those required to achieve the desired therapeutic effect and to gradually increase the dosage until the desired effect is achieved. If desired, then the effective daily dose can be divided into multiple doses for purposes of administration. Consequently, a single dose of a disclosed compositions and/or a pharmaceutical preparation comprising one or more disclosed compositions, or methods can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease, a disorder, an infection, a symptom, and/or a complication.

“Control” as used herein refers a standard or reference condition, against which results are compared. In an aspect, a control is used at the same time as a test variable or subject to provide a comparison. In an aspect, a control is a historical control that has been performed previously, a result or amount that has been previously known, or an otherwise existing record. A control may can be a positive or negative control.

As used herein, the term “diagnosed” means having been subjected to a physical examination by a person of skill, for example, a physician, and found to have a disease, a disorder, an infection, a symptom, and/or a complication that can be diagnosed or treated by one or more of the disclosed variant capsid proteins, the disclosed vectors, the disclosed nucleic acid molecules, the disclosed compositions thereof, the disclosed pharmaceutical formulations, and/or the disclosed methods. For example. “suspected of having” can mean having been subjected to a physical examination by a person of skill, for example, a physician, and found to have a condition that can likely be treated by one or more of the disclosed variant capsid proteins, the disclosed vectors, the disclosed nucleic acid molecules, the disclosed compositions thereof, the disclosed pharmaceutical formulations, and/or the disclosed methods.

The words “treat” or “treating” or “treatment” refer to therapeutic or medical treatment wherein the object is to slow down (lessen), ameliorate, and/or diminish an undesired physiological change, disease, pathological condition, or disorder in a subject. As used herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Treatment may not necessarily result in the complete clearance of an infection but may reduce or minimize complications, the side effects, and/or the progression of a disease, a disorder, an infection, a symptom, and/or a complication. The success or otherwise of treatment can be monitored by physical examination of the subject as well as cytopathological, DNA, and/or mRNA detection techniques. The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating an infection can reduce the severity of an established infection in a subject by 1%-100% as compared to a control (such as, for example, a subject not having the disease, the disorder, the infection, the symptom, and/or the complication. In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of an established disease, disorder, infection, symptom, and/or complication. In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms. It is understood that treatment does not necessarily refer to a cure or complete ablation or eradication of the disease, disorder, infection, symptom, and/or complication. However, in an aspect, treatment can refer to a cure or complete ablation or eradication of the disease, disorder, infection, symptom, and/or complication.

Methods and techniques to monitor a subject's response to a disclosed method can comprise qualitative (or subjective) means as well as quantitative (or objective) means. In an aspect, qualitative means (or subjective means) can comprise a subject's own perspective. For example, a subject can report how he/she is feeling, whether he/she has experienced improvements and/or setbacks, whether he/she has experienced an amelioration or an intensification of one or more symptoms, or a combination thereof. In an aspect, quantitative means (or objective means) can comprise methods and techniques that include, but are not limited to, the following: (i) fluid analysis (e.g., tests of a subject's fluids including but not limited to aqueous humor and vitreous humor, bile, blood, blood serum, breast milk, cerebrospinal fluid, cerumen (earwax), digestive fluids, endolymph and perilymph, female ejaculate, gastric juice, mucus (including nasal drainage and phlegm), peritoneal fluid, pleural fluid, saliva, sebum (skin oil), semen, sweat, synovial fluid, tears, vaginal secretion, vomit, and urine), (ii) imaging (e.g., ordinary x-rays, ultrasonography, radioisotope (nuclear) scanning, computed tomography (CT), magnetic resonance imaging (MRI), positron emission tomography (PET), and angiography), (iii) endoscopy (e.g., laryngoscopy, bronchoscopy, esophagoscopy, gastroscopy, GI endoscopy, coloscopy, cystoscopy, hysteroscopy, arthroscopy, laparoscopy, mediastinoscopy, and thoracoscopy), (iv) analysis of organ activity (e.g., electrocardiography (ECG), electroencephalography (EEG), and pulse oximetry), (v) biopsy (e.g., removal of tissue samples for microscopic evaluation), and (vi) genetic testing.

A “patient” refers to a subject afflicted with a disease, disorder, infection, symptom, and/or complication. In an aspect, a patient can refer to a subject that has been diagnosed with or is suspected of having a disease, disorder, infection, symptom, and/or complication. In an aspect, a patient can refer to a subject that has been diagnosed with or is suspected of having an established disease, disorder, infection, symptom, and/or complication and is seeking treatment or receiving treatment.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease, disorder, infection, symptom, and/or complication is intended. The words “prevent” and “preventing” and “prevention” also refer to prophylactic or preventative measures for protecting or precluding a subject (e.g., an individual) not having a given infection related complication from progressing to that complication. Individuals in which prevention is required include those who have an infection.

As used herein, the terms “administering” and “administration” refer to any method of providing one or more of the disclosed variant capsid proteins, the disclosed vectors, the disclosed nucleic acid molecules, the disclosed compositions thereof, the disclosed pharmaceutical formulations, and/or the disclosed methods to a subject. Such methods are well known to those skilled in the art and include, but are not limited to, the following: oral administration, transdermal administration, administration by inhalation, nasal administration, topical administration, intravaginal administration, ophthalmic administration, intraaural administration, otic administration, inter utero administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent.

As used herein, “modifying the method” can comprise modifying or changing one or more features or aspects of one or more steps of a disclosed method. For example, in an aspect, a method can be altered by changing the amount of one or more of the disclosed variant capsid proteins, the disclosed vectors, the disclosed nucleic acid molecules, the disclosed compositions thereof, the disclosed pharmaceutical formulations, or any combination thereof administered to a subject, or by changing the frequency of administration, or by changing the duration of time of administration or between administrations to a subject.

As used herein, “concurrently” means (1) simultaneously in time, or (2) at different times during the course of a common treatment schedule.

The term “contacting” as used herein refers to bringing one or more of the disclosed variant capsid proteins, the disclosed vectors, the disclosed nucleic acid molecules, the disclosed compositions thereof, the disclosed pharmaceutical formulations, or any combination thereof with a target area or intended target area (e.g., the intestinal epithelium) in such a manner that the one or more of the disclosed variant capsid proteins, the disclosed vectors, the disclosed nucleic acid molecules, the disclosed compositions thereof, the disclosed pharmaceutical formulations, or any combination thereof can exert an effect on the intended target or targeted area either directly or indirectly.

As used herein, “determining” can refer to measuring or ascertaining the presence and severity of a disease, disorder, infection, symptom, and/or complication. Methods and techniques used to determining the presence and/or severity of a disease, disorder, infection, symptom, and/or complication are typically known to the medical arts. For example, the art is familiar with the ways to identify and/or diagnose the presence, severity, or both of a disease, disorder, infection, symptom, and/or complication.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “derivative” refers to a compound having a structure derived from the structure of a parent compound (such as, e.g., a polypeptide having the sequence set forth in any of SEQ ID NO: 141-SEQ ID NO: 155 or a nucleic acid having the sequence set forth in any of SEQ ID NO:156-SEQ ID NO:170) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include fragments of a disclosed protein (e.g., SEQ ID NO: 141-SEQ ID NO:155) or nucleic acid sequence (e.g., SEQ ID NO:156-SEQ ID NO:170).

As used herein, the term “analog” refers to a compound having a structure derived from the structure of a parent compound (such as, e.g., a polypeptide having the sequence set forth in any of SEQ ID NO: 141-SEQ ID NO:155 or a nucleic acid having the sequence set forth in any of SEQ ID NO:156-SEQ ID NO:170) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds.

As used herein, AAV genomes or AAV vectors according to the invention, when present in a suitable producer cell and in the presence of AAV Rep and Cap proteins, can replicate and package into viral particles, particularly infectious viral particles.

As used herein. “adeno-associated virus” or “AAV” refers to a viral particle consisting of at least one AAV capsid protein VP1, VP2, and/or VP3, preferably all capsid proteins of a wild-type AAV, and an encapsidated polynucleotide AAV genome or AAV vector. An AAV of the invention can typically be a recombinant AAV. An AAV can be a non-naturally occurring AAV. The AAV can comprise one or more heterologous polynucleotides, i.e., polynucleotides other than wild-type AAV polynucleotides, such as transgenes. An example of a transgene is a therapeutic gene.

As used herein, “transfer plasmid” or “pTransfer” contains the viral genome. The pTransfer further comprises two ITRs, a transgene, gene of interest, and/or payload, a promoter, and one or more cis-regulatory elements (e.g., Lox sites, WPRE, poly A, etc.).

As used herein, a “transgene” is a polynucleotide encoding a gene that is delivered to a cell by a disclosed AAV vector.

A “gene” refers to a polynucleotide containing at least one open reading frame that is capable of encoding a particular gene product after being transcribed, and sometimes also translated. The term “gene” or “coding sequence” refers to a nucleotide sequence in vitro or in vivo that encodes a gene product. In some instances, the gene consists or consists essentially of coding sequence, that is, sequence that encodes the gene product. In other instances, the gene comprises additional, non-coding, sequence that permits, facilitates or directs the cellular expression machinery to express the encoded product. Such sequences can include, but are not limited to promoters, enhancers, transcriptional termination and/or poly(A) addition signals, and elements that affect transcript processing and/or stability. A gene may or may not include regions preceding and following the coding region. e.g. 5′ untranslated (5′ UTR) or “leader” sequences and 3′ UTR or “trailer” sequences, as well as intervening sequences (introns) between individual coding segments (exons).

A “gene product” is a molecule resulting from expression of a particular gene or fragment thereof. Gene products can include, for example, a polypeptide, an aptamer, an interfering RNA, an mRNA, and the like. A “gene product” can be a polypeptide, peptide, protein or interfering RNA including short interfering RNA (siRNA), miRNA or small hairpin RNA (shRNA). In an aspect, a disclosed gene product can be a therapeutic gene product, e.g., a therapeutic protein or a therapeutic RNA.

As used herein, a “therapeutic gene” refers to a gene that, when expressed, produces a therapeutic gene product that confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include, but are not limited to, genes that correct a genetic deficiency in a cell or mammal.

AAV can be replication competent or replication incompetent. By “replication competent” is meant that the virus or viral particle is infectious and capable of replication in a suitable infected cell. In an aspect, the disclosed AAV can be replication-incompetent.

As used herein, the term “viral vector” refers to a nucleic acid vector construct that includes at least one element of viral origin and has the capacity to be packaged into a viral particle. The viral vector can contain a nucleic acid (e.g., a transgene, a gene of interest, and/or a payload) encoding a polypeptide as described herein in place of non-essential viral genes. The vector and/or particle can be utilized for the purpose of transferring nucleic acids into cells either in vitro or in vivo. Numerous forms of viral vectors are known in the art.

As used herein, an “AAV virus” or “AAV viral particle” refers to a viral particle composed of at least one AAV capsid protein such as VP1 (typically by all of the capsid proteins of a wild-type AAV) and an encapsidated polynucleotide rAAV vector. If the particle comprises a heterologous polynucleotide (i.e. a polynucleotide other than a wild-type AAV genome, such as a transgene to be delivered to a mammalian cell), it is typically referred to as a “recombinant AAV vector particle” or simply a “rAAV vector”. Thus, production of a rAAV particle necessarily includes production of a rAAV vector, as such a vector is contained within a rAAV particle.

As used herein, “viral capsid polypeptide” refers to the proteinaceous shell or coat of a viral particle. A viral capsid polypeptide permits packaging or assembly of the capsid polypeptide into a viral particle that is competent for delivery of nucleic acid to the host cell. Capsids function to encapsidate, protect, transport, and release into a host cell a viral genome. Capsids are generally comprised of oligomeric structural subunits of a polypeptide of the viral capsid polypeptides.

As used herein, “encapsidated” means enclosed within a viral capsid. As an example, the AAV genome comprises three overlapping sequences which encode capsid proteins, VP1, VP2 and VP3, which start from one promoter, p40. The AAV capsid is composed of a mixture of VP1, VP2, and VP3 totaling 60 monomers arranged in icosahedral symmetry in a ratio of 1:1:10.

As used herein, “packaging” refers to a series of intracellular events that result in the assembly and encapsidation of an AAV particle.

As used herein, “payload” refers to a nucleic acid that is encapsidated within a viral vector, e.g., an AAV vector. A payload nucleic acid can encode a polypeptide, an inhibitory RNA, an antibody or antibody reagent, an oligonucleotide, or a miRNA. As used herein, a “payload” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid. In an aspect, a disclosed payload can comprise any nucleic acid that is useful for modulating the expression in a target cell transduced or contacted with the AAV particle carrying the payload. In an aspect, modulation can be by supplementation of the payload in a target cell or tissue. In an aspect, modulation can be gene replacement of the payload in a target cell or tissue. In an aspect, modulation can be by inhibition using a modulatory nucleic acid of the payload in a target cell or tissue. In an aspect, a disclosed payload can comprise a combination of coding and non-coding nucleic acid sequences, and can be codon-optimized. In an aspect, a payload can comprise one or more regulatable elements. In an aspect, a disclosed payload can encode a messenger RNA (mRNA) can be encoded by a disclosed payload. In an aspect, a disclosed payload can encode a gene therapy product. A gene therapy product can comprise a polypeptide, RNA molecule, or other gene product that, when expressed in a target cell, provides a desired therapeutic effect. In an aspect, a gene therapy product can comprise a substitute for a non-functional gene that is absent or mutated. In an aspect, a disclosed payload nucleic acid can encode a transgene having a beneficial or desirable gene product.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide can be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using computer programs commonly employed for this purpose, e.g., that are freely available on the world wide web (e.g., BLASTp or BLASTn with default settings). Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

“Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the term “corresponding to,” when used in reference to an amino acid or polynucleotide sequence means that a given amino acid or polynucleotide sequence in one polypeptide or polynucleotide molecule has structural properties, functional properties, or both that are similar relative to an amino acid or polynucleotide sequence in a similar location in another polypeptide or polynucleotide molecule. Homologues of a given polypeptide in different species “correspond to” each other, as do regions or domains of homologous polypeptides from different species. Similarly, capsid polypeptides of different serotypes of viral vectors, including but not limited to adeno-associated virus (AAV) vectors, “correspond to” each other, as do regions of such polypeptides, defined, for example by alignment of their amino acid sequences. While other alignment parameters can be used to define such regions, for the avoidance of doubt, alignment can be performed using BLAST® (Basic Local Alignment Search Tool) using default parameters.

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

“Neuron-specific promoters” are known to the art and include, but are not limited to, the synapsin I (SYN) promoter, the calcium/calmodulin-dependent protein kinase II promoter, the tubulin alpha I promoter, the neuron-specific enolase promoter, and the platelet-derived growth factor beta chain promoter.

“Liver-specific promoters” are known to the art and include, but are not limited to, the al-microglobulin/bikunin enhancer/thyroid hormone-binding globulin promoter, the human albumin (hALB) promoter, the thyroid hormone-binding globulin promoter, thyroxin binding globulin promoter, the α-1-anti-trypsin promoter, the bovine albumin (bAlb) promoter, the murine albumin (mAlb) promoter, the human α1-antitrypsin (hAAT) promoter, the ApoEhAAT promoter composed of the ApoE enhancer and the hAAT promoter, the transthyretin (TTR) promoter, the liver fatty acid binding protein promoter, the hepatitis B virus (HBV) promoter, the DC172 promoter consisting of the hAAT promoter and the α1-microglobulin enhancer, the DC190 promoter containing the human albumin promoter and the prothrombin enhancer, and other natural and synthetic liver-specific promoters.

“Muscle-specific promoters” are known to the art and include, but are not limited to, the MHCK7 promoter, the muscle creatine kinase (MCK) promoter/enhancer, the slow isoform of troponin I (TnIS) promoter, the MYODI promoter, the MYLK2 promoter, the SPc5-12 promoter, the desmin (Des) promoter, the unc45b promoter, and other natural and synthetic muscle-specific promoters.

“Skeletal muscle-specific promoters” are known to the art and include, but are not limited to, the HSA promoter, the human α-skeletal actin promoter.

“Heart-specific promoters” are known to the art and include, but art not limited to, the MYH6 promoter, the TNNI3 promoter, the cardiac troponin C (cTnC) promoter, the alpha-myosin heavy chain (α-MHC) promoter, myosin light chain 2 (MLC-2), and the MYBPC3 promoter.

As used herein, the term “immunotolerant” refers to unresponsiveness to an antigen (e.g., a vector, a therapeutic protein derived from a human, a non-human animal, a plant, or a microorganism, such as, for example, a microbial GBE. An immunotolerant promoter can reduce, ameliorate, or prevent transgene-induced immune responses that can be associated with gene therapy. Assays known in the art to measure immune responses, such as immunohistochemical detection of cytotoxic T cell responses, can be used to determine whether one or more promoters can confer immunotolerant properties.

As used herein, a “ubiquitous/constitutive promoter” refer to a promoter that allows for continual transcription of its associated gene. A ubiquitous/constitutive promoter is always active and can be used to express genes in a wide range of cells and tissues, including, but not limited to, the liver, kidney, skeletal muscle, cardiac muscle, smooth muscle, diaphragm muscle, brain, spinal cord, endothelial cells, intestinal cells, pulmonary cells (e.g., smooth muscle or epithelium), peritoneal epithelial cells, and fibroblasts. Ubiquitous/constitutive promoters include, but are not limited to, a CMV major immediate-early enhancer/chicken beta-actin promoter, a cytomegalovirus (CMV) major immediate-early promoter, an Elongation Factor 1-α (EF1α) promoter, a simian vacuolating virus 40 (SV40) promoter, an AmpR promoter, a PγK promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, a β-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous/constitutive promoters.

As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

As used herein, an “isolated” biological component (such as a nucleic acid molecule, protein, or virus) has been substantially separated or purified away from other biological components (e.g., other chromosomal and extra-chromosomal DNA and RNA, proteins and/or organelles). Nucleic acids, proteins, and/or viruses that have been “isolated” include nucleic acids, proteins, and viruses purified by standard purification methods. The term also embraces nucleic acids, proteins, and viruses prepared by recombinant expression in a host cell, as well as chemically synthesized nucleic acids or proteins. The term “isolated” (or purified) does not require absolute purity; rather, it is intended as a relative term. Thus, for example, an isolated or purified nucleic acid, protein, virus, or other active compound is one that is isolated in whole or in part from associated nucleic acids, proteins, and other contaminants. In an aspect, the term “substantially purified” refers to a nucleic acid, protein, virus or other active compound that has been isolated from a cell, cell culture medium, or other crude preparation and subjected to fractionation to remove various components of the initial preparation, such as proteins, cellular debris, and other components.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of AAV capsid proteins. It should be understood that sequence with substantial sequence identity do not necessarily have the same length and may differ in length. For example, sequences that have the same nucleotide sequence but of which one has additional nucleotides on the 3′- and/or 5′-side are 100% identical.

A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

Disclosed are the components to be used to prepare one or more of the disclosed variant capsid proteins, the disclosed vectors, the disclosed nucleic acid molecules, the disclosed compositions thereof, the disclosed pharmaceutical formulations, and/or the disclosed methods used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations. A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspects or combination of aspects of the methods of the invention.

### B. Compositions

### 1. Variant AAV Capsid Proteins

Disclosed herein is a variant AAV capsid protein. Disclosed herein is a variant AAV capsid protein having one or more substitutions in variable region IV (VR-IV). Disclosed herein is a variant AAV capsid protein having one or more substitutions in variable region VIII (VR-VIII). Disclosed herein is a variant AAV capsid protein having one or more substitutions in variable region IV and/or variable region VIII.

Disclosed herein is an adeno-associated virus (AAV) capsid protein comprising a sequence having one or more substitutions relative to the sequence set forth in SEQ ID NO:01. Disclosed herein is an adeno-associated virus (AAV) capsid protein comprising a sequence having one or more substitutions relative to the sequence set forth in SEQ ID NO:02. Disclosed herein is an adeno-associated virus (AAV) capsid protein comprising a sequence having one or more substitutions relative to the sequence set forth in SEQ ID NO:03. Disclosed herein is an adeno-associated virus (AAV) capsid protein comprising a sequence having one or more substitutions relative to the sequence set forth in SEQ ID NO:04. Disclosed herein is an adeno-associated virus (AAV) capsid protein comprising a sequence having one or more substitutions relative to the sequence set forth in SEQ ID NO:05. Disclosed herein is an adeno-associated virus (AAV) capsid protein comprising a sequence having one or more substitutions relative to the sequence set forth in SEQ ID NO:06. Disclosed herein is an adeno-associated virus (AAV) capsid protein comprising a sequence having one or more substitutions relative to the sequence set forth in SEQ ID NO:07. Disclosed herein is an adeno-associated virus (AAV) capsid protein comprising a sequence having one or more substitutions relative to the sequence set forth in SEQ ID NO:08. Disclosed herein is an adeno-associated virus (AAV) capsid protein comprising a sequence having one or more substitutions relative to the sequence set forth in SEQ ID NO:10. Disclosed herein is an adeno-associated virus (AAV) capsid protein comprising a sequence having one or more substitutions relative to the sequence set forth in SEQ ID NO:11. Disclosed herein is an adeno-associated virus (AAV) capsid protein comprising a sequence having one or more substitutions relative to the sequence set forth in SEQ ID NO:12. Disclosed herein is an adeno-associated virus (AAV) capsid protein comprising a sequence having one or more substitutions relative to the sequence set forth in SEQ ID NO:13. Disclosed herein is an adeno-associated virus (AAV) capsid protein comprising a sequence having one or more substitutions relative to the sequence set forth in SEQ ID NO:14. Disclosed herein is an adeno-associated virus (AAV) capsid protein comprising a sequence having one or more substitutions relative to the sequence set forth in SEQ ID NO:15. Disclosed herein is an adeno-associated virus (AAV) capsid protein comprising a sequence having one or more substitutions relative to the sequence set forth in SEQ ID NO:16. For example, in an aspect, a disclosed wild-type sequence (as presented below in Table 1) has one or more substitutions in one or more regions of the protein.

TABLE 1
Sequences for Wild-Type or Non-
Substituted AAV Capsid Proteins

| | Sequence for Wild- |
|---|---|
| AAV | Type Capsid Protein |
| AAV1 | SEQ ID NO: 01 |
| AAV2 | SEQ ID NO: 02 |
| AAV3b | SEQ ID NO: 03 |
| AAV4 | SEQ ID NO: 04 |
| AAV5 | SEQ ID NO: 05 |
| AAV6 | SEQ ID NO: 06 |
| AAV7 | SEQ ID NO: 07 |
| AAV8 | SEQ ID NO: 08 |
| AAV9 | SEQ ID NO: 09 |
| AAV10 | SEQ ID NO: 10 |
| AAV11 | SEQ ID NO: 11 |
| AAV12 | SEQ ID NO: 12 |
| AAV13 | SEQ ID NO: 13 |
| AAVrh8 | SEQ ID NO: 14 |
| AAVrh10 | SEQ ID NO: 15 |
| AAVrh32.33 | SEQ ID NO: 16 |
| AAVhum.8 | SEQ ID NO: 166 |
| AAV DJ | SEQ ID NO: 167 |
| AAV CAM130 | SEQ ID NO: 168 |
| AAV 1RX | SEQ ID NO: 169 |
| AAV 2i8j9 | SEQ ID NO: 170 |
| AAV 4.69 | SEQ ID NO: 171 |

In an aspect, the disclosed one or more substitutions can comprise 1 substitution, 2 substitutions, 3 substitutions, 4 substitutions, 5 substitutions, 6 substitutions, 7 substitutions, 8 substitutions, 9 substitutions, or 10 substitutions. In an aspect, the disclosed one or more substitutions can comprise 7 substitutions.

Disclosed herein is an adeno-associated virus (AAV) capsid protein comprising the sequence set forth in SEQ ID NO: 172 or SEQ ID NO: 188. Disclosed herein is an adeno-associated virus (AAV) capsid protein comprising the sequence set forth in SEQ ID NO: 173 or SEQ ID NO: 189. Disclosed herein is an adeno-associated virus (AAV) capsid protein comprising the sequence set forth in SEQ ID NO:174 or SEQ ID NO:190. Disclosed herein is an adeno-associated virus (AAV) capsid protein comprising the sequence set forth in SEQ ID NO:175 or SEQ ID NO:191. Disclosed herein is an adeno-associated virus (AAV) capsid protein comprising the sequence set forth in SEQ ID NO:176 or SEQ ID NO:192. Disclosed herein is an adeno-associated virus (AAV) capsid protein comprising the sequence set forth in SEQ ID NO:177 or SEQ ID NO:193. Disclosed herein is an adeno-associated virus (AAV) capsid protein comprising the sequence set forth in SEQ ID NO:178 or SEQ ID NO:194. Disclosed herein is an adeno-associated virus (AAV) capsid protein comprising the sequence set forth in SEQ ID NO:179 or SEQ ID NO:195. Disclosed herein is an adeno-associated virus (AAV) capsid protein comprising the sequence set forth in SEQ ID NO:180 or SEQ ID NO:196. Disclosed herein is an adeno-associated virus (AAV) capsid protein comprising the sequence set forth in SEQ ID NO: 181 or SEQ ID NO: 197. Disclosed herein is an adeno-associated virus (AAV) capsid protein comprising the sequence set forth in SEQ ID NO:182 or SEQ ID NO: 198. Disclosed herein is an adeno-associated virus (AAV) capsid protein comprising the sequence set forth in SEQ ID NO: 183 or SEQ ID NO:199. Disclosed herein is an adeno-associated virus (AAV) capsid protein comprising the sequence set forth in SEQ ID NO:184 or SEQ ID NO:200. Disclosed herein is an adeno-associated virus (AAV) capsid protein comprising the sequence set forth in SEQ ID NO:185 or SEQ ID NO:201. Disclosed herein is an adeno-associated virus (AAV) capsid protein comprising the sequence set forth in SEQ ID NO:186 or SEQ ID NO:202. Disclosed herein is an adeno-associated virus (AAV) capsid protein comprising the sequence set forth in SEQ ID NO: 187 or SEQ ID NO:203. For example, in an aspect, a disclosed AAV capsid variant can comprise the sequence set forth below in Table 2.

TABLE 2
Sequences for Variant AAV Capsid Proteins

| AAV | Sequence Identifier |
|---|---|
| AAV1 | SEQ ID NO: 172 or SEQ ID NO: 188 |
| AAV2 | SEQ ID NO: 173 or SEQ ID NO: 189 |
| AAV3b | SEQ ID NO: 174 or SEQ ID NO: 190 |
| AAV4 | SEQ ID NO: 175 or SEQ ID NO: 191 |
| AAV5 | SEQ ID NO: 176 or SEQ ID NO: 192 |
| AAV6 | SEQ ID NO: 177 or SEQ ID NO: 193 |
| AAV7 | SEQ ID NO: 178 or SEQ ID NO: 194 |
| AAV8 | SEQ ID NO: 179 or SEQ ID NO: 195 |
| AAV9 | SEQ ID NO: 180 or SEQ ID NO: 196 |
| AAV10 | SEQ ID NO: 181 or SEQ ID NO: 197 |
| AAV11 | SEQ ID NO: 182 or SEQ ID NO: 198 |
| AAV12 | SEQ ID NO: 183 or SEQ ID NO: 199 |
| AAV13 | SEQ ID NO: 184 or SEQ ID NO: 200 |
| AAVrh8 | SEQ ID NO: 185 or SEQ ID NO: 201 |
| AAVrh10 | SEQ ID NO: 186 or SEQ ID NO: 202 |
| AAVrh32.33 | SEQ ID NO: 187 or SEQ ID NO: 203 |

Disclosed herein is an adeno-associated virus (AAV) capsid protein comprising the sequence set forth in SEQ ID NO:01 but for one or more substitutions in the region of SEQ ID NO:17. Disclosed herein is an adeno-associated virus (AAV) capsid protein comprising the sequence set forth in SEQ ID NO:02 but for one or more substitutions in the region of SEQ ID NO: 19. Disclosed herein is an adeno-associated virus (AAV) capsid protein comprising the sequence set forth in SEQ ID NO:03 but for one or more substitutions in the region of SEQ ID NO:21. Disclosed herein is an adeno-associated virus (AAV) capsid protein comprising the sequence set forth in SEQ ID NO:04 but for one or more substitutions in the region of SEQ ID NO:23. Disclosed herein is an adeno-associated virus (AAV) capsid protein comprising the sequence set forth in SEQ ID NO:05 but for one or more substitutions in the region of SEQ ID NO:25. Disclosed herein is an adeno-associated virus (AAV) capsid protein comprising the sequence set forth in SEQ ID NO:06 but for one or more substitutions in the region of SEQ ID NO:27. Disclosed herein is an adeno-associated virus (AAV) capsid protein comprising the sequence set forth in SEQ ID NO:07 but for one or more substitutions in the region of SEQ ID NO:29. Disclosed herein is an adeno-associated virus (AAV) capsid protein comprising the sequence set forth in SEQ ID NO:08 but for one or more substitutions in the region of SEQ ID NO:31. Disclosed herein is an adeno-associated virus (AAV) capsid protein comprising the sequence set forth in SEQ ID NO:09 but for one or more substitutions in the region of SEQ ID NO:33. Disclosed herein is an adeno-associated virus (AAV) capsid protein comprising the sequence set forth in SEQ ID NO:10 but for one or more substitutions in the region of SEQ ID NO:35. Disclosed herein is an adeno-associated virus (AAV) capsid protein comprising the sequence set forth in SEQ ID NO:11 but for one or more substitutions in the region of SEQ ID NO:37. Disclosed herein is an adeno-associated virus (AAV) capsid protein comprising the sequence set forth in SEQ ID NO: 12 but for one or more substitutions in the region of SEQ ID NO:39. Disclosed herein is an adeno-associated virus (AAV) capsid protein comprising the sequence set forth in SEQ ID NO:13 but for one or more substitutions in the region of SEQ ID NO:41. Disclosed herein is an adeno-associated virus (AAV) capsid protein comprising the sequence set forth in SEQ ID NO:14 but for one or more substitutions in the region of SEQ ID NO:43. Disclosed herein is an adeno-associated virus (AAV) capsid protein comprising the sequence set forth in SEQ ID NO:15 but for one or more substitutions in the region of SEQ ID NO:45. Disclosed herein is an adeno-associated virus (AAV) capsid protein comprising the sequence set forth in SEQ ID NO:16 but for one or more substitutions in the region of SEQ ID NO:47.

Disclosed herein is an adeno-associated virus (AAV) capsid protein comprising the sequence set forth in SEQ ID NO:01 but for one or more substitutions in the region of SEQ ID NO:18. Disclosed herein is an adeno-associated virus (AAV) capsid protein comprising the sequence set forth in SEQ ID NO:02 but for one or more substitutions in the region of SEQ ID NO:20. Disclosed herein is an adeno-associated virus (AAV) capsid protein comprising the sequence set forth in SEQ ID NO:03 but for one or more substitutions in the region of SEQ ID NO:22. Disclosed herein is an adeno-associated virus (AAV) capsid protein comprising the sequence set forth in SEQ ID NO:04 but for one or more substitutions in the region of SEQ ID NO:24. Disclosed herein is an adeno-associated virus (AAV) capsid protein comprising the sequence set forth in SEQ ID NO:05 but for one or more substitutions in the region of SEQ ID NO:26. Disclosed herein is an adeno-associated virus (AAV) capsid protein comprising the sequence set forth in SEQ ID NO:06 but for one or more substitutions in the region of SEQ ID NO:28. Disclosed herein is an adeno-associated virus (AAV) capsid protein comprising the sequence set forth in SEQ ID NO:07 but for one or more substitutions in the region of SEQ ID NO:30. Disclosed herein is an adeno-associated virus (AAV) capsid protein comprising the sequence set forth in SEQ ID NO:08 but for one or more substitutions in the region of SEQ ID NO:32. Disclosed herein is an adeno-associated virus (AAV) capsid protein comprising the sequence set forth in SEQ ID NO:09 but for one or more substitutions in the region of SEQ ID NO:34. Disclosed herein is an adeno-associated virus (AAV) capsid protein comprising the sequence set forth in SEQ ID NO:10 but for one or more substitutions in the region of SEQ ID NO:36. Disclosed herein is an adeno-associated virus (AAV) capsid protein comprising the sequence set forth in SEQ ID NO: 11 but for one or more substitutions in the region of SEQ ID NO:38. Disclosed herein is an adeno-associated virus (AAV) capsid protein comprising the sequence set forth in SEQ ID NO:12 but for one or more substitutions in the region of SEQ ID NO:40. Disclosed herein is an adeno-associated virus (AAV) capsid protein comprising the sequence set forth in SEQ ID NO: 13 but for one or more substitutions in the region of SEQ ID NO:42. Disclosed herein is an adeno-associated virus (AAV) capsid protein comprising the sequence set forth in SEQ ID NO:14 but for one or more substitutions in the region of SEQ ID NO:44. Disclosed herein is an adeno-associated virus (AAV) capsid protein comprising the sequence set forth in SEQ ID NO: 15 but for one or more substitutions in the region of SEQ ID NO:46. Disclosed herein is an adeno-associated virus (AAV) capsid protein comprising the sequence set forth in SEQ ID NO:16 but for one or more substitutions in the region of SEQ ID NO:48.

Disclosed herein is an adeno-associated virus (AAV) capsid protein comprising a sequence having at least 95% identity to the sequence set forth in SEQ ID NO:01 with one or more substitutions in variable region IV (VR-IV). Disclosed herein is an adeno-associated virus (AAV) capsid protein comprising a sequence having at least 95% identity to the sequence set forth in SEQ ID NO:02 with one or more substitutions in variable region IV (VR-IV). Disclosed herein is an adeno-associated virus (AAV) capsid protein comprising a sequence having at least 95% identity to the sequence set forth in SEQ ID NO:03 with one or more substitutions in variable region IV (VR-IV). Disclosed herein is an adeno-associated virus (AAV) capsid protein comprising a sequence having at least 95% identity to the sequence set forth in SEQ ID NO:04 with one or more substitutions in variable region IV (VR-IV). Disclosed herein is an adeno-associated virus (AAV) capsid protein comprising a sequence having at least 95% identity to the sequence set forth in SEQ ID NO:05 with one or more substitutions in variable region IV (VR-IV). Disclosed herein is an adeno-associated virus (AAV) capsid protein comprising a sequence having at least 95% identity to the sequence set forth in SEQ ID NO:06 with one or more substitutions in variable region IV (VR-IV). Disclosed herein is an adeno-associated virus (AAV) capsid protein comprising a sequence having at least 95% identity to the sequence set forth in SEQ ID NO:07 with one or more substitutions in variable region IV (VR-IV). Disclosed herein is an adeno-associated virus (AAV) capsid protein comprising a sequence having at least 95% identity to the sequence set forth in SEQ ID NO:08 with one or more substitutions in variable region IV (VR-IV). Disclosed herein is an adeno-associated virus (AAV) capsid protein comprising a sequence having at least 95% identity to the sequence set forth in SEQ ID NO:09 with one or more substitutions in variable region IV (VR-IV). Disclosed herein is an adeno-associated virus (AAV) capsid protein comprising a sequence having at least 95% identity to the sequence set forth in SEQ ID NO:10 with one or more substitutions in variable region IV (VR-IV). Disclosed herein is an adeno-associated virus (AAV) capsid protein comprising a sequence having at least 95% identity to the sequence set forth in SEQ ID NO:11 with one or more substitutions in variable region IV (VR-IV). Disclosed herein is an adeno-associated virus (AAV) capsid protein comprising a sequence having at least 95% identity to the sequence set forth in SEQ ID NO:12 with one or more substitutions in variable region IV (VR-IV). Disclosed herein is an adeno-associated virus (AAV) capsid protein comprising a sequence having at least 95% identity to the sequence set forth in SEQ ID NO: 13 with one or more substitutions in variable region IV (VR-IV). Disclosed herein is an adeno-associated virus (AAV) capsid protein comprising a sequence having at least 95% identity to the sequence set forth in SEQ ID NO:14 with one or more substitutions in variable region IV (VR-IV).

Disclosed herein is an adeno-associated virus (AAV) capsid protein comprising a sequence having at least 95% identity to the sequence set forth in SEQ ID NO:15 with one or more substitutions in variable region IV (VR-IV). Disclosed herein is an adeno-associated virus (AAV) capsid protein comprising a sequence having at least 95% identity to the sequence set forth in SEQ ID NO: 16 with one or more substitutions in variable region IV (VR-IV). Disclosed herein is an adeno-associated virus (AAV) capsid protein comprising a sequence having at least 95% identity to the sequence set forth in SEQ ID NO:01 with one or more substitutions in variable region VIII (VR-VIII). Disclosed herein is an adeno-associated virus (AAV) capsid protein comprising a sequence having at least 95% identity to the sequence set forth in SEQ ID NO:02 with one or more substitutions in variable region VIII (VR-VIII). Disclosed herein is an adeno-associated virus (AAV) capsid protein comprising a sequence having at least 95% identity to the sequence set forth in SEQ ID NO:03 with one or more substitutions in variable region VIII (VR-VIII). Disclosed herein is an adeno-associated virus (AAV) capsid protein comprising a sequence having at least 95% identity to the sequence set forth in SEQ ID NO:04 with one or more substitutions in variable region VIII (VR-VIII). Disclosed herein is an adeno-associated virus (AAV) capsid protein comprising a sequence having at least 95% identity to the sequence set forth in SEQ ID NO:05 with one or more substitutions in variable region VIII (VR-VIII). Disclosed herein is an adeno-associated virus (AAV) capsid protein comprising a sequence having at least 95% identity to the sequence set forth in SEQ ID NO:06 with one or more substitutions in variable region VIII (VR-VIII). Disclosed herein is an adeno-associated virus (AAV) capsid protein comprising a sequence having at least 95% identity to the sequence set forth in SEQ ID NO:07 with one or more substitutions in variable region VIII (VR-VIII). Disclosed herein is an adeno-associated virus (AAV) capsid protein comprising a sequence having at least 95% identity to the sequence set forth in SEQ ID NO:08 with one or more substitutions in variable region VIII (VR-VIII). Disclosed herein is an adeno-associated virus (AAV) capsid protein comprising a sequence having at least 95% identity to the sequence set forth in SEQ ID NO:09 with one or more substitutions in variable region VIII (VR-VIII). Disclosed herein is an adeno-associated virus (AAV) capsid protein comprising a sequence having at least 95% identity to the sequence set forth in SEQ ID NO: 10 with one or more substitutions in variable region VIII (VR-VIII). Disclosed herein is an adeno-associated virus (AAV) capsid protein comprising a sequence having at least 95% identity to the sequence set forth in SEQ ID NO:11 with one or more substitutions in variable region VIII (VR-VIII). Disclosed herein is an adeno-associated virus (AAV) capsid protein comprising a sequence having at least 95% identity to the sequence set forth in SEQ ID NO: 12 with one or more substitutions in variable region VIII (VR-VIII). Disclosed herein is an adeno-associated virus (AAV) capsid protein comprising a sequence having at least 95% identity to the sequence set forth in SEQ ID NO:13 with one or more substitutions in variable region VIII (VR-VIII). Disclosed herein is an adeno-associated virus (AAV) capsid protein comprising a sequence having at least 95% identity to the sequence set forth in SEQ ID NO:14 with one or more substitutions in variable region VIII (VR-VIII). Disclosed herein is an adeno-associated virus (AAV) capsid protein comprising a sequence having at least 95% identity to the sequence set forth in SEQ ID NO: 15 with one or more substitutions in variable region VIII (VR-VIII). Disclosed herein is an adeno-associated virus (AAV) capsid protein comprising a sequence having at least 95% identity to the sequence set forth in SEQ ID NO: 16 with one or more substitutions in variable region VIII (VR-VIII).

Disclosed herein is an adeno-associated virus (AAV) capsid protein comprising a sequence having one or more substitutions in variable region IV (VR-IV) and variable region VIII (VR-VIII) relative to SEQ ID NO:01. Disclosed herein is an adeno-associated virus (AAV) capsid protein comprising a sequence having one or more substitutions in variable region IV (VR-IV) and variable region VIII (VR-VIII) relative to SEQ ID NO:02. Disclosed herein is an adeno-associated virus (AAV) capsid protein comprising a sequence having one or more substitutions in variable region IV (VR-IV) and variable region VIII (VR-VIII) relative to SEQ ID NO:03. Disclosed herein is an adeno-associated virus (AAV) capsid protein comprising a sequence having one or more substitutions in variable region IV (VR-IV) and variable region VIII (VR-VIII) relative to SEQ ID NO:04. Disclosed herein is an adeno-associated virus (AAV) capsid protein comprising a sequence having one or more substitutions in variable region IV (VR-IV) and variable region VIII (VR-VIII) relative to SEQ ID NO:05. Disclosed herein is an adeno-associated virus (AAV) capsid protein comprising a sequence having one or more substitutions in variable region IV (VR-IV) and variable region VIII (VR-VIII) relative to SEQ ID NO:06. Disclosed herein is an adeno-associated virus (AAV) capsid protein comprising a sequence having one or more substitutions in variable region IV (VR-IV) and variable region VIII (VR-VIII) relative to SEQ ID NO:07. Disclosed herein is an adeno-associated virus (AAV) capsid protein comprising a sequence having one or more substitutions in variable region IV (VR-IV) and variable region VIII (VR-VIII) relative to SEQ ID NO:08. Disclosed herein is an adeno-associated virus (AAV) capsid protein comprising a sequence having one or more substitutions in variable region IV (VR-IV) and variable region VIII (VR-VIII) relative to SEQ ID NO:09. Disclosed herein is an adeno-associated virus (AAV) capsid protein comprising a sequence having one or more substitutions in variable region IV (VR-IV) and variable region VIII (VR-VIII) relative to SEQ ID NO:10. Disclosed herein is an adeno-associated virus (AAV) capsid protein comprising a sequence having one or more substitutions in variable region IV (VR-IV) and variable region VIII (VR-VIII) relative to SEQ ID NO:11. Disclosed herein is an adeno-associated virus (AAV) capsid protein comprising a sequence having one or more substitutions in variable region IV (VR-IV) and variable region VIII (VR-VIII) relative to SEQ ID NO:12. Disclosed herein is an adeno-associated virus (AAV) capsid protein comprising a sequence having one or more substitutions in variable region IV (VR-IV) and variable region VIII (VR-VIII) relative to SEQ ID NO:13. Disclosed herein is an adeno-associated virus (AAV) capsid protein comprising a sequence having one or more substitutions in variable region IV (VR-IV) and variable region VIII (VR-VIII) relative to SEQ ID NO:14. Disclosed herein is an adeno-associated virus (AAV) capsid protein comprising a sequence having one or more substitutions in variable region IV (VR-IV) and variable region VIII (VR-VIII) relative to SEQ ID NO:15. Disclosed herein is an adeno-associated virus (AAV) capsid protein comprising a sequence having one or more substitutions in variable region IV (VR-IV) and variable region VIII (VR-VIII) relative to SEQ ID NO:16.

In an aspect, a disclosed variant AAV capsid protein can be used to improve and/or can be used to enhance gene transfer efficiency in intestinal epithelial cells and/or colonic epithelial cells. In an aspect, a disclosed variant AAV capsid protein can be used to improve and/or can be used to enhance gene transfer efficiency in intestinal epithelial cells and/or colonic epithelial cells. In an aspect, a disclosed variant AAV capsid protein can be used to improve and/or can be used to enhance gene transfer efficiency in intestinal epithelial cells and/or colonic epithelial cells. In an aspect, a disclosed variant AAV capsid protein can be used to improve and/or can be used to enhance the quality of the subject's life when compared to a pre-treatment level. In an aspect, a disclosed variant AAV capsid protein can be used to diminish and/or decrease one or more symptoms associated with and/or related to the subject's diseases and/or disorder.

In an aspect, a disclosed variant AAV capsid protein can be used to prevent an undesired physiological change, disease, pathological condition, or disorder from occurring in the subject. In an aspect, a disclosed variant AAV capsid protein can be used to inhibit a physiological change, disease, pathological condition, or disorder, i.e., arresting its development, in the subject. In an aspect, a disclosed variant AAV capsid protein can be used to relieve a physiological change, disease, pathological condition, or disorder. i.e., causing regression of the disease, in the subject. In an aspect, a disclosed variant AAV capsid protein can be used to improve the subject's quality of life by at least 50% when compared to the subject's pre-treatment quality of life.

In an aspect, a disclosed variant AAV capsid protein can be used to improve and/or can be used to alleviate one or more gastrointestinal tract manifestations of cystic fibrosis in the subject. In an aspect, a GI tract manifestation of cystic fibrosis can comprise mucous inspissation, dysmotility, meconium ileus (MI), constipation, distal intestinal obstruction syndrome (DIOS), gastroesophageal reflux disease (GERD), small bowel bacterial overgrowth, or any combination thereof.

In an aspect, a disclosed variant AAV capsid protein can be used to improve and/or can be used to alleviate nutritional failure in the subject. In an aspect, a disclosed variant AAV capsid protein can be used to improve the malabsorption of fat, protein, and/or fat-soluble vitamins in the subject.

In an aspect, a disclosed variant AAV capsid protein can be used to improve pancreatic insufficiency in the subject. In an aspect, a disclosed variant AAV capsid protein can be used to decrease the risk for recurrent pancreatitis in the subject. In an aspect, a disclosed variant AAV capsid protein can be used to reduce the risk of developing chronic lung disease in the subject.

In an aspect, a disclosed variant AAV capsid protein can be used to reduce the risk of developing a pulmonary infection and/or progressive pulmonary infection in the subject. In an aspect, a disclosed variant AAV capsid protein can be used to repair diseased and/or dysfunctional intestinal epithelium and/or colonic epithelium.

In an aspect, a disclosed variant AAV capsid protein can be used to reduce intestinal and/or colonic inflammation in the subject. In an aspect, a disclosed variant AAV capsid protein can be used to treat a subject in need thereof. In an aspect, a disclosed variant AAV capsid protein can be used in a method of delivering gene therapy to a subject in need thereof. In an aspect, a disclosed variant AAV capsid protein can be incorporated into a disclosed AAV capsid. In an aspect, a disclosed variant AAV capsid protein can demonstrate improved tropism for one or more cell types and/or one or more tissue types (such as, for example, intestinal epithelium and/or colonic epithelium). In an aspect, a disclosed variant AAV capsid protein can exhibit improved transduction efficiency and/or properties when introduced to one or more cell types and/or one or more tissue types. In an aspect, AAVcc47 can efficiently transduce the cells of the intestinal epithelium and/or colonic epithelium. In an aspect, AAVcc47 can outperform wild-type AAV9 in the cells of the intestinal epithelium and/or colonic epithelium.

### 2. Isolated Nucleic Acid Molecules

Disclosed herein is an isolated nucleic acid molecule comprising a nucleic acid sequence encoding a disclosed variant AAV capsid protein. Disclosed herein is an isolated nucleic acid molecule comprising a nucleic acid sequence encoding a variant AAV capsid protein. Disclosed herein is an isolated nucleic acid molecule comprising a nucleic acid sequence encoding a variant AAV capsid protein having one or more substitutions in variable region IV (VR-IV). Disclosed herein is an isolated nucleic acid molecule comprising a nucleic acid sequence encoding a variant AAV capsid protein having one or more substitutions in variable region VIII (VR-VIII). Disclosed herein is an isolated nucleic acid molecule comprising a nucleic acid sequence encoding a variant AAV capsid protein having one or more substitutions in variable region IV and/or variable region VIII.

*Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO:01. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO:02. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO:03. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO:04. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO:05. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO:06. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO:07. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO:08. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO: 10. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO:11. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO:12. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO:13. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO: 14. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO: 15. Disclosed herein is an isolated nucleic acid molecule comprising a nucleic acid sequence encoding an adeno-associated virus (AAV) capsid protein comprising a sequence having one or more substitutions relative to the sequence set forth in SEQ ID NO:16. For example, in an aspect, a disclosed wild-type sequence (Table 1) has one or more substitutions in one or more regions of the protein. In an aspect, the disclosed one or more substitutions can comprise 1 substitution, 2 substitutions, 3 substitutions, 4 substitutions, 5 substitutions, 6 substitutions, 7 substitutions, 8 substitutions, 9 substitutions, or 10 substitutions. In an aspect, the disclosed one or more substitutions can comprise 7 substitutions.

Disclosed herein is an isolated nucleic acid molecule comprising a nucleic acid sequence encoding an adeno-associated virus (AAV) capsid protein comprising the sequence set forth in SEQ ID NO:172 or SEQ ID NO:188. Disclosed herein is an isolated nucleic acid molecule comprising a nucleic acid sequence encoding an adeno-associated virus (AAV) capsid protein comprising the sequence set forth in SEQ ID NO:173 or SEQ ID NO:189. Disclosed herein is an adeno-associated virus (AAV) capsid protein comprising the sequence set forth in SEQ ID NO:174 or SEQ ID NO:190. Disclosed herein is an isolated nucleic acid molecule comprising a nucleic acid sequence encoding an adeno-associated virus (AAV) capsid protein comprising the sequence set forth in SEQ ID NO:175 or SEQ ID NO:191. Disclosed herein is an isolated nucleic acid molecule comprising a nucleic acid sequence encoding an adeno-associated virus (AAV) capsid protein comprising the sequence set forth in SEQ ID NO:176 or SEQ ID NO:192. Disclosed herein is an isolated nucleic acid molecule comprising a nucleic acid sequence encoding an adeno-associated virus (AAV) capsid protein comprising the sequence set forth in SEQ ID NO:177 or SEQ ID NO: 193. Disclosed herein is an isolated nucleic acid molecule comprising a nucleic acid sequence encoding an adeno-associated virus (AAV) capsid protein comprising the sequence set forth in SEQ ID NO: 178 or SEQ ID NO:194. Disclosed herein is an isolated nucleic acid molecule comprising a nucleic acid sequence encoding an adeno-associated virus (AAV) capsid protein comprising the sequence set forth in SEQ ID NO:179 or SEQ ID NO:195. Disclosed herein is an isolated nucleic acid molecule comprising a nucleic acid sequence encoding an adeno-associated virus (AAV) capsid protein comprising the sequence set forth in SEQ ID NO:180 or SEQ ID NO: 196. Disclosed herein is an isolated nucleic acid molecule comprising a nucleic acid sequence encoding an adeno-associated virus (AAV) capsid protein comprising the sequence set forth in SEQ ID NO:181 or SEQ ID NO:197. Disclosed herein is an isolated nucleic acid molecule comprising a nucleic acid sequence encoding an adeno-associated virus (AAV) capsid protein comprising the sequence set forth in SEQ ID NO:182 or SEQ ID NO:198. Disclosed herein is an isolated nucleic acid molecule comprising a nucleic acid sequence encoding an adeno-associated virus (AAV) capsid protein comprising the sequence set forth in SEQ ID NO:183 or SEQ ID NO:199. Disclosed herein is an isolated nucleic acid molecule comprising a nucleic acid sequence encoding an adeno-associated virus (AAV) capsid protein comprising the sequence set forth in SEQ ID NO:184 or SEQ ID NO:200. Disclosed herein is an isolated nucleic acid molecule comprising a nucleic acid sequence encoding an adeno-associated virus (AAV) capsid protein comprising the sequence set forth in SEQ ID NO: 185 or SEQ ID NO:201. Disclosed herein is an isolated nucleic acid molecule comprising a nucleic acid sequence encoding an adeno-associated virus (AAV) capsid protein comprising the sequence set forth in SEQ ID NO:186 or SEQ ID NO:202. Disclosed herein is an isolated nucleic acid molecule comprising a nucleic acid sequence encoding an adeno-associated virus (AAV) capsid protein comprising the sequence set forth in SEQ ID NO: 187 or SEQ ID NO:203. For example, in an aspect, a disclosed encoded AAV capsid variant can comprise the sequence set forth in Table 2.

Disclosed herein is an isolated nucleic acid molecule comprising a nucleic acid sequence encoding an adeno-associated virus (AAV) capsid protein comprising the sequence set forth in SEQ ID NO:01 but for one or more substitutions in the region of SEQ ID NO: 17. Disclosed herein is an isolated nucleic acid molecule comprising a nucleic acid sequence encoding an adeno-associated virus (AAV) capsid protein comprising the sequence set forth in SEQ ID NO:02 but for one or more substitutions in the region of SEQ ID NO:19. Disclosed herein is an isolated nucleic acid molecule comprising a nucleic acid sequence encoding an adeno-associated virus (AAV) capsid protein comprising the sequence set forth in SEQ ID NO:03 but for one or more substitutions in the region of SEQ ID NO:21. Disclosed herein is an isolated nucleic acid molecule comprising a nucleic acid sequence encoding an adeno-associated virus (AAV) capsid protein comprising the sequence set forth in SEQ ID NO:04 but for one or more substitutions in the region of SEQ ID NO:23. Disclosed herein is an isolated nucleic acid molecule comprising a nucleic acid sequence encoding an adeno-associated virus (AAV) capsid protein comprising the sequence set forth in SEQ ID NO:05 but for one or more substitutions in the region of SEQ ID NO:25. Disclosed herein is an isolated nucleic acid molecule comprising a nucleic acid sequence encoding an adeno-associated virus (AAV) capsid protein comprising the sequence set forth in SEQ ID NO:06 but for one or more substitutions in the region of SEQ ID NO:27. Disclosed herein is an isolated nucleic acid molecule comprising a nucleic acid sequence encoding an adeno-associated virus (AAV) capsid protein comprising the sequence set forth in SEQ ID NO:07 but for one or more substitutions in the region of SEQ ID NO:29. Disclosed herein is an isolated nucleic acid molecule comprising a nucleic acid sequence encoding an adeno-associated virus (AAV) capsid protein comprising the sequence set forth in SEQ ID NO:08 but for one or more substitutions in the region of SEQ ID NO:31. Disclosed herein is an isolated nucleic acid molecule comprising a nucleic acid sequence encoding an adeno-associated virus (AAV) capsid protein comprising the sequence set forth in SEQ ID NO:09 but for one or more substitutions in the region of SEQ ID NO:33. Disclosed herein is an isolated nucleic acid molecule comprising a nucleic acid sequence encoding an adeno-associated virus (AAV) capsid protein comprising the sequence set forth in SEQ ID NO: 10 but for one or more substitutions in the region of SEQ ID NO:35. Disclosed herein is an isolated nucleic acid molecule comprising a nucleic acid sequence encoding an adeno-associated virus (AAV) capsid protein comprising the sequence set forth in SEQ ID NO:11 but for one or more substitutions in the region of SEQ ID NO:37. Disclosed herein is an isolated nucleic acid molecule comprising a nucleic acid sequence encoding an adeno-associated virus (AAV) capsid protein comprising the sequence set forth in SEQ ID NO:12 but for one or more substitutions in the region of SEQ ID NO:39. Disclosed herein is an isolated nucleic acid molecule comprising a nucleic acid sequence encoding an adeno-associated virus (AAV) capsid protein comprising the sequence set forth in SEQ ID NO: 13 but for one or more substitutions in the region of SEQ ID NO:41. Disclosed herein is an isolated nucleic acid molecule comprising a nucleic acid sequence encoding an adeno-associated virus (AAV) capsid protein comprising the sequence set forth in SEQ ID NO:14 but for one or more substitutions in the region of SEQ ID NO:43. Disclosed herein is an isolated nucleic acid molecule comprising a nucleic acid sequence encoding an adeno-associated virus (AAV) capsid protein comprising the sequence set forth in SEQ ID NO:15 but for one or more substitutions in the region of SEQ ID NO:45. Disclosed herein is an isolated nucleic acid molecule comprising a nucleic acid sequence encoding an adeno-associated virus (AAV) capsid protein comprising the sequence set forth in SEQ ID NO: 16 but for one or more substitutions in the region of SEQ ID NO:47.

Disclosed herein is an isolated nucleic acid molecule comprising a nucleic acid sequence encoding an adeno-associated virus (AAV) capsid protein comprising the sequence set forth in SEQ ID NO:01 but for one or more substitutions in the region of SEQ ID NO:18. Disclosed herein is an isolated nucleic acid molecule comprising a nucleic acid sequence encoding an adeno-associated virus (AAV) capsid protein comprising the sequence set forth in SEQ ID NO:02 but for one or more substitutions in the region of SEQ ID NO:20. Disclosed herein is an isolated nucleic acid molecule comprising a nucleic acid sequence encoding an adeno-associated virus (AAV) capsid protein comprising the sequence set forth in SEQ ID NO:03 but for one or more substitutions in the region of SEQ ID NO:22. Disclosed herein is an isolated nucleic acid molecule comprising a nucleic acid sequence encoding an adeno-associated virus (AAV) capsid protein comprising the sequence set forth in SEQ ID NO:04 but for one or more substitutions in the region of SEQ ID NO:24. Disclosed herein is an isolated nucleic acid molecule comprising a nucleic acid sequence encoding an adeno-associated virus (AAV) capsid protein comprising the sequence set forth in SEQ ID NO:05 but for one or more substitutions in the region of SEQ ID NO:26. Disclosed herein is an isolated nucleic acid molecule comprising a nucleic acid sequence encoding an adeno-associated virus (AAV) capsid protein comprising the sequence set forth in SEQ ID NO:06 but for one or more substitutions in the region of SEQ ID NO:28. Disclosed herein is an isolated nucleic acid molecule comprising a nucleic acid sequence encoding an adeno-associated virus (AAV) capsid protein comprising the sequence set forth in SEQ ID NO:07 but for one or more substitutions in the region of SEQ ID NO:30. Disclosed herein is an isolated nucleic acid molecule comprising a nucleic acid sequence encoding an adeno-associated virus (AAV) capsid protein comprising the sequence set forth in SEQ ID NO:08 but for one or more substitutions in the region of SEQ ID NO:32. Disclosed herein is an isolated nucleic acid molecule comprising a nucleic acid sequence encoding an adeno-associated virus (AAV) capsid protein comprising the sequence set forth in SEQ ID NO:09 but for one or more substitutions in the region of SEQ ID NO:34. Disclosed herein is an isolated nucleic acid molecule comprising a nucleic acid sequence encoding an adeno-associated virus (AAV) capsid protein comprising the sequence set forth in SEQ ID NO: 10 but for one or more substitutions in the region of SEQ ID NO:36. Disclosed herein is an isolated nucleic acid molecule comprising a nucleic acid sequence encoding an adeno-associated virus (AAV) capsid protein comprising the sequence set forth in SEQ ID NO:11 but for one or more substitutions in the region of SEQ ID NO:38. Disclosed herein is an isolated nucleic acid molecule comprising a nucleic acid sequence encoding an adeno-associated virus (AAV) capsid protein comprising the sequence set forth in SEQ ID NO:12 but for one or more substitutions in the region of SEQ ID NO:40. Disclosed herein is an isolated nucleic acid molecule comprising a nucleic acid sequence encoding an adeno-associated virus (AAV) capsid protein comprising the sequence set forth in SEQ ID NO: 13 but for one or more substitutions in the region of SEQ ID NO:42. Disclosed herein is an isolated nucleic acid molecule comprising a nucleic acid sequence encoding an adeno-associated virus (AAV) capsid protein comprising the sequence set forth in SEQ ID NO: 14 but for one or more substitutions in the region of SEQ ID NO:44. Disclosed herein is an isolated nucleic acid molecule comprising a nucleic acid sequence encoding an adeno-associated virus (AAV) capsid protein comprising the sequence set forth in SEQ ID NO: 15 but for one or more substitutions in the region of SEQ ID NO:46. Disclosed herein is an isolated nucleic acid molecule comprising a nucleic acid sequence encoding an adeno-associated virus (AAV) capsid protein comprising the sequence set forth in SEQ ID NO:16 but for one or more substitutions in the region of SEQ ID NO:48.

Disclosed herein is an isolated nucleic acid molecule comprising a nucleic acid sequence encoding an adeno-associated virus (AAV) capsid protein comprising a sequence having at least 95% identity to the sequence set forth in SEQ ID NO:01 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:02 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:03 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:04 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:05 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:06 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:07 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:08 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:09 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO: 10 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO: 11 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:12 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:13 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO: 14 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:15 with one or more substitutions in variable region IV (VR-IV). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:16 with one or more substitutions in variable region IV (VR-IV).

Disclosed herein is an isolated nucleic acid molecule comprising a nucleic acid sequence encoding an adeno-associated virus (AAV) capsid protein comprising a sequence having at least 95% identity to the sequence set forth in SEQ ID NO:01 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:02 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:03 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:04 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:05 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:06 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:07 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:08 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:09 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:10 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:11 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:12 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO: 13 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:14 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:15 with one or more substitutions in variable region VIII (VR-VIII). Disclosed herein is an isolated nucleic acid molecule comprising a nucleic acid sequence encoding an adeno-associated virus (AAV) capsid protein comprising a sequence having at least 95% identity to the sequence set forth in SEQ ID NO:16 with one or more substitutions in variable region VIII (VR-VIII).

Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:01. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:02. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:03. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:04. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:05. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:06. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:07. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:08. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:09. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO: 10. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:11. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:12. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:13. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO: 14. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:15. Disclosed herein is an isolated nucleic acid molecule comprising a nucleic acid sequence encoding an adeno-associated virus (AAV) capsid protein comprising a sequence having one or more substitutions in variable region IV (VR-IV) and variable region VIII (VR-VIII) relative to SEQ ID NO:16.

In an aspect, a disclosed isolated nucleic acid molecule can comprise the sequence for one or more regulatory elements. For example, a disclosed regulatory element can comprise a promoter operably linked to a disclosed isolated nucleic acid molecule, wherein the promoter drives the expression of a disclosed variant capsid protein, a disclosed encoded polypeptide, a disclosed encoded therapeutic agent, or both.

In an aspect, a disclosed isolated nucleic acid molecule can comprise the sequence for at least one of poly peptide, a glycopeptide, a polysaccharide, a glycolipid, a lipid, or a nucleic acid polymer, or a combination thereof. In an aspect, a disclosed isolated nucleic acid molecule can comprise the sequence for at least one therapeutic agent. In an aspect, a disclosed therapeutic agent can be an oligonucleotide therapeutic agent. In an aspect, a disclosed oligonucleotide therapeutic agent can be a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed therapeutic agent can be an ASO or an RNAi. In an aspect, a disclosed nucleic acid-based molecule can comprise one or more modifications at any position applicable. In an aspect, a disclosed therapeutic agent can comprise a CRISPR-based endonuclease (e.g., Cas9). In an aspect, a disclosed CRISPR-based endonuclease can be derived from a CRISPR/Cas type I, type II, or type III system.

In an aspect, a disclosed encoded variant AAV capsid protein can be used to improve and/or can be used to enhance gene transfer efficiency in intestinal epithelial cells and/or colonic epithelial cells. In an aspect, a disclosed encoded variant AAV capsid protein can be used to improve and/or can be used to enhance gene transfer efficiency in intestinal epithelial cells and/or colonic epithelial cells. In an aspect, a disclosed encoded variant AAV capsid protein can be used to improve and/or can be used to enhance gene transfer efficiency in intestinal epithelial cells and/or colonic epithelial cells. In an aspect, a disclosed encoded variant AAV capsid protein can be used to improve and/or can be used to enhance the quality of the subject's life when compared to a pre-treatment level.

In an aspect, a disclosed encoded variant AAV capsid protein can be used to diminish and/or decrease one or more symptoms associated with and/or related to the subject's diseases and/or disorder. In an aspect, a disclosed encoded variant AAV capsid protein can be used to prevent an undesired physiological change, disease, pathological condition, or disorder from occurring in the subject. In an aspect, a disclosed encoded variant AAV capsid protein can be used to inhibit a physiological change, disease, pathological condition, or disorder, i.e., arresting its development, in the subject. In an aspect, a disclosed encoded variant AAV capsid protein can be used to relieve a physiological change, disease, pathological condition, or disorder, i.e., causing regression of the disease, in the subject. In an aspect, a disclosed encoded variant AAV capsid protein can be used to improve the subject's quality of life by at least 50% when compared to the subject's pre-treatment quality of life.

In an aspect, a disclosed encoded variant AAV capsid protein can be used to improve and/or can be used to alleviate one or more gastrointestinal tract manifestations of cystic fibrosis in the subject. In an aspect, a GI tract manifestation of cystic fibrosis can comprise mucous inspissation, dysmotility, meconium ileus (MI), constipation, distal intestinal obstruction syndrome (DIOS), gastroesophageal reflux disease (GERD), small bowel bacterial overgrowth, or any combination thereof.

In an aspect, a disclosed encoded variant AAV capsid protein can be used to improve and/or can be used to alleviate nutritional failure in the subject. In an aspect, a disclosed encoded variant AAV capsid protein can be used to improve the malabsorption of fat, protein, and/or fat-soluble vitamins in the subject.

In an aspect, a disclosed encoded variant AAV capsid can be used to improve pancreatic insufficiency in the subject. In an aspect, a disclosed encoded variant AAV capsid protein can be used to decrease the risk for recurrent pancreatitis in the subject.

In an aspect, a disclosed encoded variant AAV capsid protein can be used to reduce the risk of developing chronic lung disease in the subject. In an aspect, a disclosed encoded variant AAV capsid protein can be used to reduce the risk of developing a pulmonary infection and/or progressive pulmonary infection in the subject.

In an aspect, a disclosed encoded variant AAV capsid protein can be used to repair diseased and/or dysfunctional intestinal epithelium and/or colonic epithelium. In an aspect, a disclosed encoded variant AAV capsid protein can be used to reduce intestinal and/or colonic inflammation in the subject. In an aspect, a disclosed encoded variant AAV capsid protein can be used to treat a subject in need thereof. In an aspect, a disclosed encoded variant AAV capsid protein can be used in a method of delivering gene therapy to a subject in need thereof.

In an aspect, a disclosed variant encoded AAV capsid protein can be incorporated into a disclosed AAV capsid.

In an aspect, a disclosed variant encoded AAV capsid protein can demonstrate improved tropism for one or more cell types and/or one or more tissue types. In an aspect, a disclosed variant AAV capsid protein can exhibit improved transduction efficiency and/or properties when introduced to one or more cell types and/or one or more tissue types. In an aspect, AAVcc47 can efficiently transduce the cells of the intestinal epithelium and/or colonic epithelium. In an aspect, AAVcc47 can outperform wild-type AAV9 in the cells of the intestinal epithelium and/or colonic epithelium.

### 3. AAV Cap Gene

Disclosed herein is an AAV cap gene comprising a disclosed nucleic acid molecule. Disclosed herein is an AAV cap gene comprising a disclosed nucleic acid sequence for a variant AAV capsid protein. Disclosed herein is a recombinant AAV cap gene encoding a variant AAV capsid protein. Disclosed herein is a recombinant AAV cap gene encoding a variant AAV capsid protein having one or more substitutions in variable region IV (VR-IV). Disclosed herein is a recombinant AAV cap gene encoding a variant AAV capsid protein having one or more substitutions in variable region VIII (VR-VIII). Disclosed herein is a recombinant AAV cap gene encoding a variant AAV capsid protein having one or more substitutions in variable region IV and/or variable region VIII.

Disclosed herein is a recombinant AAV cap gene encoding an adeno-associated virus (AAV) capsid protein comprising a sequence having one or more substitutions relative to the sequence set forth in SEQ ID NO:01. Disclosed herein is a recombinant AAV cap gene encoding an adeno-associated virus (AAV) capsid protein comprising a sequence having one or more substitutions relative to the sequence set forth in SEQ ID NO:02. Disclosed herein is a recombinant AAV cap gene encoding an adeno-associated virus (AAV) capsid protein comprising a sequence having one or more substitutions relative to the sequence set forth in SEQ ID NO:03. Disclosed herein is a recombinant AAV cap gene encoding an adeno-associated virus (AAV) capsid protein comprising a sequence having one or more substitutions relative to the sequence set forth in SEQ ID NO:04. Disclosed herein is a recombinant AAV cap gene encoding an adeno-associated virus (AAV) capsid protein comprising a sequence having one or more substitutions relative to the sequence set forth in SEQ ID NO:05. Disclosed herein is a recombinant AAV cap gene encoding an adeno-associated virus (AAV) capsid protein comprising a sequence having one or more substitutions relative to the sequence set forth in SEQ ID NO:06. Disclosed herein is a recombinant AAV cap gene encoding an adeno-associated virus (AAV) capsid protein comprising a sequence having one or more substitutions relative to the sequence set forth in SEQ ID NO:07. Disclosed herein is a recombinant AAV cap gene encoding an adeno-associated virus (AAV) capsid protein comprising a sequence having one or more substitutions relative to the sequence set forth in SEQ ID NO:08. Disclosed herein is a recombinant AAV cap gene encoding an adeno-associated virus (AAV) capsid protein comprising a sequence having one or more substitutions relative to the sequence set forth in SEQ ID NO:10. Disclosed herein is a recombinant AAV cap gene encoding an adeno-associated virus (AAV) capsid protein comprising a sequence having one or more substitutions relative to the sequence set forth in SEQ ID NO:11. Disclosed herein is a recombinant AAV cap gene encoding an adeno-associated virus (AAV) capsid protein comprising a sequence having one or more substitutions relative to the sequence set forth in SEQ ID NO:12. Disclosed herein is a recombinant AAV cap gene encoding an adeno-associated virus (AAV) capsid protein comprising a sequence having one or more substitutions relative to the sequence set forth in SEQ ID NO:13. Disclosed herein is a recombinant AAV cap gene encoding an adeno-associated virus (AAV) capsid protein comprising a sequence having one or more substitutions relative to the sequence set forth in SEQ ID NO: 14. Disclosed herein is a recombinant AAV cap gene encoding an adeno-associated virus (AAV) capsid protein comprising a sequence having one or more substitutions relative to the sequence set forth in SEQ ID NO:15. Disclosed herein is a recombinant AAV cap gene encoding an adeno-associated virus (AAV) capsid protein comprising a sequence having one or more substitutions relative to the sequence set forth in SEQ ID NO: 16. For example, in an aspect, a disclosed wild-type sequence (as presented below in Table 1) has one or more substitutions in one or more regions of the protein.

In an aspect, the disclosed one or more substitutions can comprise 1 substitution, 2 substitutions, 3 substitutions, 4 substitutions, 5 substitutions, 6 substitutions, 7 substitutions, 8 substitutions, 9 substitutions, or 10 substitutions. In an aspect, the disclosed one or more substitutions can comprise 7 substitutions.

Disclosed herein is a recombinant AAV cap gene encoding an adeno-associated virus (AAV) capsid protein comprising the sequence set forth in SEQ ID NO:172 or SEQ ID NO: 188. Disclosed herein is a recombinant AAV cap gene encoding an adeno-associated virus (AAV) capsid protein comprising the sequence set forth in SEQ ID NO:173 or SEQ ID NO:189. Disclosed herein is a recombinant AAV cap gene encoding an adeno-associated virus (AAV) capsid protein comprising the sequence set forth in SEQ ID NO:174 or SEQ ID NO:190. Disclosed herein is a recombinant AAV cap gene encoding an adeno-associated virus (AAV) capsid protein comprising the sequence set forth in SEQ ID NO: 175 or SEQ ID NO:191. Disclosed herein is a recombinant AAV cap gene encoding an adeno-associated virus (AAV) capsid protein comprising the sequence set forth in SEQ ID NO:176 or SEQ ID NO:192. Disclosed herein is a recombinant AAV cap gene encoding an adeno-associated virus (AAV) capsid protein comprising the sequence set forth in SEQ ID NO:177 or SEQ ID NO:193. Disclosed herein is a recombinant AAV cap gene encoding an adeno-associated virus (AAV) capsid protein comprising the sequence set forth in SEQ ID NO:178 or SEQ ID NO:194. Disclosed herein is a recombinant AAV cap gene encoding an adeno-associated virus (AAV) capsid protein comprising the sequence set forth in SEQ ID NO:179 or SEQ ID NO:195. Disclosed herein is a recombinant AAV cap gene encoding an adeno-associated virus (AAV) capsid protein comprising the sequence set forth in SEQ ID NO:180 or SEQ ID NO:196. Disclosed herein is a recombinant AAV cap gene encoding an adeno-associated virus (AAV) capsid protein comprising the sequence set forth in SEQ ID NO:181 or SEQ ID NO:197. Disclosed herein is a recombinant AAV cap gene encoding an adeno-associated virus (AAV) capsid protein comprising the sequence set forth in SEQ ID NO:182 or SEQ ID NO:198. Disclosed herein is a recombinant AAV cap gene encoding an adeno-associated virus (AAV) capsid protein comprising the sequence set forth in SEQ ID NO:183 or SEQ ID NO:199. Disclosed herein is a recombinant AAV cap gene encoding an adeno-associated virus (AAV) capsid protein comprising the sequence set forth in SEQ ID NO:184 or SEQ ID NO:200. Disclosed herein is a recombinant AAV cap gene encoding an adeno-associated virus (AAV) capsid protein comprising the sequence set forth in SEQ ID NO:185 or SEQ ID NO:201. Disclosed herein is a recombinant AAV cap gene encoding an adeno-associated virus (AAV) capsid protein comprising the sequence set forth in SEQ ID NO:186 or SEQ ID NO:202. Disclosed herein is a recombinant AAV cap gene encoding an adeno-associated virus (AAV) capsid protein comprising the sequence set forth in SEQ ID NO:187 or SEQ ID NO:203. For example, in an aspect, a disclosed AAV capsid variant can comprise the sequence set forth below in Table 2.

Disclosed herein is a recombinant AAV cap gene encoding an adeno-associated virus (AAV) capsid protein comprising the sequence set forth in SEQ ID NO:01 but for one or more substitutions in the region of SEQ ID NO: 17. Disclosed herein is a recombinant AAV cap gene encoding an adeno-associated virus (AAV) capsid protein comprising the sequence set forth in SEQ ID NO:02 but for one or more substitutions in the region of SEQ ID NO:19. Disclosed herein is a recombinant AAV cap gene encoding an adeno-associated virus (AAV) capsid protein comprising the sequence set forth in SEQ ID NO:03 but for one or more substitutions in the region of SEQ ID NO:21. Disclosed herein is a recombinant AAV cap gene encoding an adeno-associated virus (AAV) capsid protein comprising the sequence set forth in SEQ ID NO:04 but for one or more substitutions in the region of SEQ ID NO:23. Disclosed herein is a recombinant AAV cap gene encoding an adeno-associated virus (AAV) capsid protein comprising the sequence set forth in SEQ ID NO:05 but for one or more substitutions in the region of SEQ ID NO:25. Disclosed herein is a recombinant AAV cap gene encoding an adeno-associated virus (AAV) capsid protein comprising the sequence set forth in SEQ ID NO:06 but for one or more substitutions in the region of SEQ ID NO:27. Disclosed herein is a recombinant AAV cap gene encoding an adeno-associated virus (AAV) capsid protein comprising the sequence set forth in SEQ ID NO:07 but for one or more substitutions in the region of SEQ ID NO:29. Disclosed herein is a recombinant AAV cap gene encoding an adeno-associated virus (AAV) capsid protein comprising the sequence set forth in SEQ ID NO:08 but for one or more substitutions in the region of SEQ ID NO:31. Disclosed herein is a recombinant AAV cap gene encoding an adeno-associated virus (AAV) capsid protein comprising the sequence set forth in SEQ ID NO:09 but for one or more substitutions in the region of SEQ ID NO:33. Disclosed herein is a recombinant AAV cap gene encoding an adeno-associated virus (AAV) capsid protein comprising the sequence set forth in SEQ ID NO:10 but for one or more substitutions in the region of SEQ ID NO:35. Disclosed herein is a recombinant AAV cap gene encoding an adeno-associated virus (AAV) capsid protein comprising the sequence set forth in SEQ ID NO:11 but for one or more substitutions in the region of SEQ ID NO:37. Disclosed herein is a recombinant AAV cap gene encoding an adeno-associated virus (AAV) capsid protein comprising the sequence set forth in SEQ ID NO:12 but for one or more substitutions in the region of SEQ ID NO:39. Disclosed herein is a recombinant AAV cap gene encoding an adeno-associated virus (AAV) capsid protein comprising the sequence set forth in SEQ ID NO:13 but for one or more substitutions in the region of SEQ ID NO:41. Disclosed herein is a recombinant AAV cap gene encoding an adeno-associated virus (AAV) capsid protein comprising the sequence set forth in SEQ ID NO:14 but for one or more substitutions in the region of SEQ ID NO:43. Disclosed herein is a recombinant AAV cap gene encoding an adeno-associated virus (AAV) capsid protein comprising the sequence set forth in SEQ ID NO:15 but for one or more substitutions in the region of SEQ ID NO:45. Disclosed herein is a recombinant AAV cap gene encoding an adeno-associated virus (AAV) capsid protein comprising the sequence set forth in SEQ ID NO:16 but for one or more substitutions in the region of SEQ ID NO:47.

Disclosed herein is a recombinant AAV cap gene encoding an adeno-associated virus (AAV) capsid protein comprising the sequence set forth in SEQ ID NO:01 but for one or more substitutions in the region of SEQ ID NO:18. Disclosed herein is a recombinant AAV cap gene encoding an adeno-associated virus (AAV) capsid protein comprising the sequence set forth in SEQ ID NO:02 but for one or more substitutions in the region of SEQ ID NO:20. Disclosed herein is a recombinant AAV cap gene encoding an adeno-associated virus (AAV) capsid protein comprising the sequence set forth in SEQ ID NO:03 but for one or more substitutions in the region of SEQ ID NO:22. Disclosed herein is a recombinant AAV cap gene encoding an adeno-associated virus (AAV) capsid protein comprising the sequence set forth in SEQ ID NO:04 but for one or more substitutions in the region of SEQ ID NO:24. Disclosed herein is a recombinant AAV cap gene encoding an adeno-associated virus (AAV) capsid protein comprising the sequence set forth in SEQ ID NO:05 but for one or more substitutions in the region of SEQ ID NO:26. Disclosed herein is a recombinant AAV cap gene encoding an adeno-associated virus (AAV) capsid protein comprising the sequence set forth in SEQ ID NO:06 but for one or more substitutions in the region of SEQ ID NO:28. Disclosed herein is a recombinant AAV cap gene encoding an adeno-associated virus (AAV) capsid protein comprising the sequence set forth in SEQ ID NO:07 but for one or more substitutions in the region of SEQ ID NO:30. Disclosed herein is a recombinant AAV cap gene encoding an adeno-associated virus (AAV) capsid protein comprising the sequence set forth in SEQ ID NO:08 but for one or more substitutions in the region of SEQ ID NO:32. Disclosed herein is a recombinant AAV cap gene encoding an adeno-associated virus (AAV) capsid protein comprising the sequence set forth in SEQ ID NO:09 but for one or more substitutions in the region of SEQ ID NO:34. Disclosed herein is a recombinant AAV cap gene encoding an adeno-associated virus (AAV) capsid protein comprising the sequence set forth in SEQ ID NO:10 but for one or more substitutions in the region of SEQ ID NO:36. Disclosed herein is a recombinant AAV cap gene encoding an adeno-associated virus (AAV) capsid protein comprising the sequence set forth in SEQ ID NO:11 but for one or more substitutions in the region of SEQ ID NO:38. Disclosed herein is a recombinant AAV cap gene encoding an adeno-associated virus (AAV) capsid protein comprising the sequence set forth in SEQ ID NO:12 but for one or more substitutions in the region of SEQ ID NO:40. Disclosed herein is a recombinant AAV cap gene encoding an adeno-associated virus (AAV) capsid protein comprising the sequence set forth in SEQ ID NO:13 but for one or more substitutions in the region of SEQ ID NO:42. Disclosed herein is a recombinant AAV cap gene encoding an adeno-associated virus (AAV) capsid protein comprising the sequence set forth in SEQ ID NO: 14 but for one or more substitutions in the region of SEQ ID NO:44. Disclosed herein is a recombinant AAV cap gene encoding an adeno-associated virus (AAV) capsid protein comprising the sequence set forth in SEQ ID NO:15 but for one or more substitutions in the region of SEQ ID NO:46. Disclosed herein is a recombinant AAV cap gene encoding an adeno-associated virus (AAV) capsid protein comprising the sequence set forth in SEQ ID NO: 16 but for one or more substitutions in the region of SEQ ID NO:48.

Disclosed herein is a recombinant AAV cap gene encoding an adeno-associated virus (AAV) capsid protein comprising a sequence having at least 95% identity to the sequence set forth in SEQ ID NO:01 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:02 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:03 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:04 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:05 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:06 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:07 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:08 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:09 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:10 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:11 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:12 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:13 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:14 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:15 with one or more substitutions in variable region IV (VR-IV). Disclosed herein is a recombinant AAV cap gene encoding an adeno-associated virus (AAV) capsid protein comprising a sequence having at least 95% identity to the sequence set forth in SEQ ID NO:16 with one or more substitutions in variable region IV (VR-IV).

Disclosed herein is a recombinant AAV cap gene encoding an adeno-associated virus (AAV) capsid protein comprising a sequence having at least 95% identity to the sequence set forth in SEQ ID NO:01 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:02 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:03 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:04 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:05 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:06 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:07 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:08 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:09 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:10 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:11 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:12 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:13 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:14 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:15 with one or more substitutions in variable region VIII (VR-VIII). Disclosed herein is a recombinant AAV cap gene encoding an adeno-associated virus (AAV) capsid protein comprising a sequence having at least 95% identity to the sequence set forth in SEQ ID NO:16 with one or more substitutions in variable region VIII (VR-VIII).

Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO:01. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO:02. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO:03. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO:04. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO:05. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO:06. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO:07. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO:08. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO:09. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO:10. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO: 11. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO:12. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO: 13. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO: 14. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO:15. Disclosed herein is a recombinant AAV cap gene encoding an adeno-associated virus (AAV) capsid protein comprising a sequence having one or more substitutions in variable region IV (VR-IV) and variable region VIII (VR-VIII) relative to SEQ ID NO: 16.

In an aspect, a disclosed encoded variant AAV capsid protein can be used to improve and/or can be used to enhance gene transfer efficiency in intestinal epithelial cells and/or colonic epithelial cells. In an aspect, a disclosed encoded variant AAV capsid protein can be used to improve and/or can be used to enhance gene transfer efficiency in intestinal epithelial cells and/or colonic epithelial cells. In an aspect, a disclosed encoded variant AAV capsid protein can be used to improve and/or can be used to enhance gene transfer efficiency in intestinal epithelial cells and/or colonic epithelial cells. In an aspect, a disclosed encoded variant AAV capsid protein can be used to improve and/or can be used to enhance the quality of the subject's life when compared to a pre-treatment level. In an aspect, a disclosed encoded variant AAV capsid protein can be used to diminish and/or decrease one or more symptoms associated with and/or related to the subject's diseases and/or disorder. In an aspect, a disclosed encoded variant AAV capsid protein can be used to prevent an undesired physiological change, disease, pathological condition, or disorder from occurring in the subject. In an aspect, a disclosed encoded variant AAV capsid protein can be used to inhibit a physiological change, disease, pathological condition, or disorder. i.e., arresting its development, in the subject. In an aspect, a disclosed encoded variant AAV capsid protein can be used to relieve a physiological change, disease, pathological condition, or disorder, i.e., causing regression of the disease, in the subject. In an aspect, a disclosed encoded variant AAV capsid protein can be used to improve the subject's quality of life by at least 50% when compared to the subject's pre-treatment quality of life.

In an aspect, a disclosed encoded variant AAV capsid protein can be used to improve and/or can be used to alleviate one or more gastrointestinal tract manifestations of cystic fibrosis in the subject. In an aspect, a GI tract manifestation of cystic fibrosis can comprise mucous inspissation, dysmotility, meconium ileus (MI), constipation, distal intestinal obstruction syndrome (DIOS), gastroesophageal reflux disease (GERD), small bowel bacterial overgrowth, or any combination thereof.

In an aspect, a disclosed encoded variant AAV capsid protein can be used to improve and/or can be used to alleviate nutritional failure in the subject. In an aspect, a disclosed encoded variant AAV capsid protein can be used to improve the malabsorption of fat, protein, and/or fat-soluble vitamins in the subject. In an aspect, a disclosed encoded variant AAV capsid protein can be used to improve the malabsorption of fat, protein, and/or fat-soluble vitamins in the subject. In an aspect, a disclosed encoded variant AAV capsid protein can be used to improve pancreatic insufficiency in the subject. In an aspect, a disclosed encoded variant AAV capsid protein can be used to decrease the risk for recurrent pancreatitis in the subject. In an aspect, a disclosed encoded variant AAV capsid protein can be used to reduce the risk of developing chronic lung disease in the subject. In an aspect, a disclosed encoded variant AAV capsid protein can be used to reduce the risk of developing a pulmonary infection and/or progressive pulmonary infection in the subject.

In an aspect, a disclosed encoded variant AAV capsid protein can be used to repair diseased and/or dysfunctional intestinal epithelium and/or colonic epithelium. In an aspect, a disclosed encoded variant AAV capsid protein can be used to reduce intestinal and/or colonic inflammation in the subject. In an aspect, a disclosed encoded variant AAV capsid protein can be used to treat a subject in need thereof. In an aspect, a disclosed encoded variant AAV capsid protein can be used in a method of delivering gene therapy to a subject in need thereof. In an aspect, a disclosed encoded variant AAV capsid protein can be incorporated into a disclose AAV capsid. In an aspect, a disclosed encoded variant AAV capsid can be incorporated into a disclosed AAV capsid.

In an aspect, a disclosed encoded variant AAV capsid protein can demonstrate improved tropism for one or more cell types and/or one or more tissue types. In an aspect, a disclosed encoded variant AAV capsid protein can exhibit improved transduction efficiency and/or properties when introduced to one or more cell types and/or one or more tissue types. In an aspect, AAVcc47 can efficiently transduce the cells of the intestinal epithelium and/or colonic epithelium. In an aspect. AAVcc47 can outperform wild-type AAV9 in the cells of the intestinal epithelium and/or colonic epithelium.

### 4. Cells

Disclosed herein is a cell comprising a disclosed nucleic acid molecule. Disclosed herein is a cell comprising a disclosed AAV having a variant capsid protein. Disclosed herein is a cell comprising a disclosed capsid having a disclosed variant. Disclosed herein is a cell comprising a disclosed viral vector. Disclosed herein is a cell comprising a rAAV vector particle. Disclosed herein are cells used to perform and/or effect a disclosed method. Disclosed herein are cells used to perform and/or effect a disclosed method of directed evolution of the AAV capsid protein. Disclosed herein are cells used to perform and/or effect a disclosed method of generating AAV particles. Disclosed herein are cells used to perform and/or effect a disclosed method of delivering a payload. Disclosed herein are producer cells capable of generating AAV having a disclosed AAV capsid variant.

### 5. Vectors and Vector Particles

Disclosed herein is a vector comprising a disclosed isolated nucleic acid molecule. Disclosed herein is a vector comprising a disclosed AAV capsid variant. Disclosed herein is an AAV vector comprising a disclosed isolated nucleic acid molecule. Disclosed herein is a AAV vector comprising a disclosed AAV capsid variant. Disclosed herein is a AAV vector comprising a capsid having a disclosed AAV capsid variant. Disclosed herein is a AAV vector for use in a disclosed method including methods of delivering a payload and methods of treating a subject in need thereof. Disclosed herein is a viral vector encoding a disclosed AAV capsid polypeptide. Disclosed herein is a rAAV vector particle comprising a disclosed AAV capsid polypeptide.

Disclosed herein is a rAAV vector particle comprising a nucleic acid sequence encoding a transgene, and, optionally, a transgene encoding a payload. In an aspect, a disclosed transgene can be operably linked to a disclosed nucleic acid sequence encoding a disclosed variant AAV capsid. In an aspect, a disclosed transgene can be provided on a second nucleic acid not operably linked to a disclosed nucleic acid encoding a disclosed variant AAV capsid.

Disclosed herein is an AAV vector comprising a variant AAV capsid protein having one or more substitutions in variable region IV (VR-IV). Disclosed herein is an AAV vector comprising a variant AAV capsid protein having one or more substitutions in variable region VIII (VR-VIII). Disclosed herein is an AAV vector comprising a variant AAV capsid protein having one or more substitutions in variable region IV and/or variable region VIII. Disclosed herein is an AAV vector comprising an adeno-associated virus (AAV) capsid protein comprising a sequence having one or more substitutions relative to the sequence set forth in SEQ ID NO:01. Disclosed herein is an AAV vector comprising an adeno-associated virus (AAV) capsid protein comprising a sequence having one or more substitutions relative to the sequence set forth in SEQ ID NO:02. Disclosed herein is an AAV vector comprising an adeno-associated virus (AAV) capsid protein comprising a sequence having one or more substitutions relative to the sequence set forth in SEQ ID NO:03. Disclosed herein is an AAV vector comprising an adeno-associated virus (AAV) capsid protein comprising a sequence having one or more substitutions relative to the sequence set forth in SEQ ID NO:04. Disclosed herein is an AAV vector comprising an adeno-associated virus (AAV) capsid protein comprising a sequence having one or more substitutions relative to the sequence set forth in SEQ ID NO:05. Disclosed herein is an AAV vector comprising an adeno-associated virus (AAV) capsid protein comprising a sequence having one or more substitutions relative to the sequence set forth in SEQ ID NO:06. Disclosed herein is an AAV vector comprising an adeno-associated virus (AAV) capsid protein comprising a sequence having one or more substitutions relative to the sequence set forth in SEQ ID NO:07. Disclosed herein is an AAV vector comprising an adeno-associated virus (AAV) capsid protein comprising a sequence having one or more substitutions relative to the sequence set forth in SEQ ID NO:08. Disclosed herein is an AAV vector comprising an adeno-associated virus (AAV) capsid protein comprising a sequence having one or more substitutions relative to the sequence set forth in SEQ ID NO:10. Disclosed herein is an AAV vector comprising an adeno-associated virus (AAV) capsid protein comprising a sequence having one or more substitutions relative to the sequence set forth in SEQ ID NO:11. Disclosed herein is an AAV vector comprising an adeno-associated virus (AAV) capsid protein comprising a sequence having one or more substitutions relative to the sequence set forth in SEQ ID NO:12. Disclosed herein is an AAV vector comprising an adeno-associated virus (AAV) capsid protein comprising a sequence having one or more substitutions relative to the sequence set forth in SEQ ID NO: 13. Disclosed herein is an AAV vector comprising an adeno-associated virus (AAV) capsid protein comprising a sequence having one or more substitutions relative to the sequence set forth in SEQ ID NO: 14. Disclosed herein is an AAV vector comprising an adeno-associated virus (AAV) capsid protein comprising a sequence having one or more substitutions relative to the sequence set forth in SEQ ID NO:15. Disclosed herein is an AAV vector comprising an adeno-associated virus (AAV) capsid protein comprising a sequence having one or more substitutions relative to the sequence set forth in SEQ ID NO: 16. For example, in an aspect, a disclosed wild-type sequence (Table 1) has one or more substitutions in one or more regions of the protein.

In an aspect, the disclosed one or more substitutions can comprise 1 substitution, 2 substitutions, 3 substitutions, 4 substitutions, 5 substitutions, 6 substitutions, 7 substitutions, 8 substitutions, 9 substitutions, or 10 substitutions. In an aspect, the disclosed one or more substitutions can comprise 7 substitutions.

Disclosed herein is an AAV vector comprising an adeno-associated virus (AAV) capsid protein comprising the sequence set forth in SEQ ID NO: 172 or SEQ ID NO: 188. Disclosed herein is an AAV vector comprising an adeno-associated virus (AAV) capsid protein comprising the sequence set forth in SEQ ID NO:173 or SEQ ID NO:189. Disclosed herein is an AAV vector comprising an adeno-associated virus (AAV) capsid protein comprising the sequence set forth in SEQ ID NO: 174 or SEQ ID NO:190. Disclosed herein is an AAV vector comprising an adeno-associated virus (AAV) capsid protein comprising the sequence set forth in SEQ ID NO:175 or SEQ ID NO:191. Disclosed herein is an AAV vector comprising an adeno-associated virus (AAV) capsid protein comprising the sequence set forth in SEQ ID NO:176 or SEQ ID NO:192. Disclosed herein is an AAV vector comprising an adeno-associated virus (AAV) capsid protein comprising the sequence set forth in SEQ ID NO: 177 or SEQ ID NO: 193. Disclosed herein is an AAV vector comprising an adeno-associated virus (AAV) capsid protein comprising the sequence set forth in SEQ ID NO:178 or SEQ ID NO:194. Disclosed herein is an AAV vector comprising an adeno-associated virus (AAV) capsid protein comprising the sequence set forth in SEQ ID NO:179 or SEQ ID NO:195. Disclosed herein is an AAV vector comprising an adeno-associated virus (AAV) capsid protein comprising the sequence set forth in SEQ ID NO:180 or SEQ ID NO:196. Disclosed herein is an AAV vector comprising an adeno-associated virus (AAV) capsid protein comprising the sequence set forth in SEQ ID NO: 181 or SEQ ID NO: 197. Disclosed herein is an AAV vector comprising an adeno-associated virus (AAV) capsid protein comprising the sequence set forth in SEQ ID NO:182 or SEQ ID NO:198. Disclosed herein is an AAV vector comprising an adeno-associated virus (AAV) capsid protein comprising the sequence set forth in SEQ ID NO:183 or SEQ ID NO:199. Disclosed herein is an AAV vector comprising an adeno-associated virus (AAV) capsid protein comprising the sequence set forth in SEQ ID NO:184 or SEQ ID NO:200. Disclosed herein is an AAV vector comprising an adeno-associated virus (AAV) capsid protein comprising the sequence set forth in SEQ ID NO:185 or SEQ ID NO:201. Disclosed herein is an AAV vector comprising an adeno-associated virus (AAV) capsid protein comprising the sequence set forth in SEQ ID NO:186 or SEQ ID NO:202. Disclosed herein is an AAV vector comprising an adeno-associated virus (AAV) capsid protein comprising the sequence set forth in SEQ ID NO: 187 or SEQ ID NO:203. For example, in an aspect, a disclosed AAV capsid variant can comprise the sequence set forth in Table 2.

Disclosed herein is an AAV vector comprising an adeno-associated virus (AAV) capsid protein comprising the sequence set forth in SEQ ID NO:01 but for one or more substitutions in the region of SEQ ID NO:17. Disclosed herein is an AAV vector comprising an adeno-associated virus (AAV) capsid protein comprising the sequence set forth in SEQ ID NO:02 but for one or more substitutions in the region of SEQ ID NO:19. Disclosed herein is an AAV vector comprising an adeno-associated virus (AAV) capsid protein comprising the sequence set forth in SEQ ID NO:03 but for one or more substitutions in the region of SEQ ID NO:21. Disclosed herein is an AAV vector comprising an adeno-associated virus (AAV) capsid protein comprising the sequence set forth in SEQ ID NO:04 but for one or more substitutions in the region of SEQ ID NO:23. Disclosed herein is an AAV vector comprising an adeno-associated virus (AAV) capsid protein comprising the sequence set forth in SEQ ID NO:05 but for one or more substitutions in the region of SEQ ID NO:25. Disclosed herein is an AAV vector comprising an adeno-associated virus (AAV) capsid protein comprising the sequence set forth in SEQ ID NO:06 but for one or more substitutions in the region of SEQ ID NO:27. Disclosed herein is an AAV vector comprising an adeno-associated virus (AAV) capsid protein comprising the sequence set forth in SEQ ID NO:07 but for one or more substitutions in the region of SEQ ID NO:29. Disclosed herein is an AAV vector comprising an adeno-associated virus (AAV) capsid protein comprising the sequence set forth in SEQ ID NO:08 but for one or more substitutions in the region of SEQ ID NO:31. Disclosed herein is an AAV vector comprising an adeno-associated virus (AAV) capsid protein comprising the sequence set forth in SEQ ID NO:09 but for one or more substitutions in the region of SEQ ID NO:33. Disclosed herein is an AAV vector comprising an adeno-associated virus (AAV) capsid protein comprising the sequence set forth in SEQ ID NO:10 but for one or more substitutions in the region of SEQ ID NO:35. Disclosed herein is an AAV vector comprising an adeno-associated virus (AAV) capsid protein comprising the sequence set forth in SEQ ID NO:11 but for one or more substitutions in the region of SEQ ID NO:37. Disclosed herein is an AAV vector comprising an adeno-associated virus (AAV) capsid protein comprising the sequence set forth in SEQ ID NO:12 but for one or more substitutions in the region of SEQ ID NO:39. Disclosed herein is an AAV vector comprising an adeno-associated virus (AAV) capsid protein comprising the sequence set forth in SEQ ID NO:13 but for one or more substitutions in the region of SEQ ID NO:41. Disclosed herein is an AAV vector comprising an adeno-associated virus (AAV) capsid protein comprising the sequence set forth in SEQ ID NO:14 but for one or more substitutions in the region of SEQ ID NO:43. Disclosed herein is an AAV vector comprising an adeno-associated virus (AAV) capsid protein comprising the sequence set forth in SEQ ID NO:15 but for one or more substitutions in the region of SEQ ID NO:45. Disclosed herein is an AAV vector comprising an adeno-associated virus (AAV) capsid protein comprising the sequence set forth in SEQ ID NO:16 but for one or more substitutions in the region of SEQ ID NO:47.

Disclosed herein is an AAV vector comprising an adeno-associated virus (AAV) capsid protein comprising the sequence set forth in SEQ ID NO:01 but for one or more substitutions in the region of SEQ ID NO:18. Disclosed herein is an AAV vector comprising an adeno-associated virus (AAV) capsid protein comprising the sequence set forth in SEQ ID NO:02 but for one or more substitutions in the region of SEQ ID NO:20. Disclosed herein is an AAV vector comprising an adeno-associated virus (AAV) capsid protein comprising the sequence set forth in SEQ ID NO:03 but for one or more substitutions in the region of SEQ ID NO:22. Disclosed herein is an AAV vector comprising an adeno-associated virus (AAV) capsid protein comprising the sequence set forth in SEQ ID NO:04 but for one or more substitutions in the region of SEQ ID NO:24. Disclosed herein is an AAV vector comprising an adeno-associated virus (AAV) capsid protein comprising the sequence set forth in SEQ ID NO:05 but for one or more substitutions in the region of SEQ ID NO:26. Disclosed herein is an AAV vector comprising an adeno-associated virus (AAV) capsid protein comprising the sequence set forth in SEQ ID NO:06 but for one or more substitutions in the region of SEQ ID NO:28. Disclosed herein is an AAV vector comprising an adeno-associated virus (AAV) capsid protein comprising the sequence set forth in SEQ ID NO:07 but for one or more substitutions in the region of SEQ ID NO:30. Disclosed herein is an AAV vector comprising an adeno-associated virus (AAV) capsid protein comprising the sequence set forth in SEQ ID NO:08 but for one or more substitutions in the region of SEQ ID NO:32. Disclosed herein is an AAV vector comprising an adeno-associated virus (AAV) capsid protein comprising the sequence set forth in SEQ ID NO:09 but for one or more substitutions in the region of SEQ ID NO:34. Disclosed herein is an AAV vector comprising an adeno-associated virus (AAV) capsid protein comprising the sequence set forth in SEQ ID NO:10 but for one or more substitutions in the region of SEQ ID NO:36. Disclosed herein is an AAV vector comprising an adeno-associated virus (AAV) capsid protein comprising the sequence set forth in SEQ ID NO:11 but for one or more substitutions in the region of SEQ ID NO:38. Disclosed herein is an AAV vector comprising an adeno-associated virus (AAV) capsid protein comprising the sequence set forth in SEQ ID NO:12 but for one or more substitutions in the region of SEQ ID NO:40. Disclosed herein is an AAV vector comprising an adeno-associated virus (AAV) capsid protein comprising the sequence set forth in SEQ ID NO:13 but for one or more substitutions in the region of SEQ ID NO:42. Disclosed herein is an AAV vector comprising an adeno-associated virus (AAV) capsid protein comprising the sequence set forth in SEQ ID NO:14 but for one or more substitutions in the region of SEQ ID NO:44. Disclosed herein is an AAV vector comprising an adeno-associated virus (AAV) capsid protein comprising the sequence set forth in SEQ ID NO:15 but for one or more substitutions in the region of SEQ ID NO:46. Disclosed herein is an AAV vector comprising an adeno-associated virus (AAV) capsid protein comprising the sequence set forth in SEQ ID NO:16 but for one or more substitutions in the region of SEQ ID NO:48.

Disclosed herein is an AAV vector comprising an adeno-associated virus (AAV) capsid protein comprising a sequence having at least 95% identity to the sequence set forth in SEQ ID NO:01 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO:02 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO:03 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO:04 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO:05 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO:06 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO:07 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO:08 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO:09 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO: 10 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO:11 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO:12 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO: 13 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO:14 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO: 15 with one or more substitutions in variable region IV (VR-IV). Disclosed herein is an AAV vector comprising an adeno-associated virus (AAV) capsid protein comprising a sequence having at least 95% identity to the sequence set forth in SEQ ID NO: 16 with one or more substitutions in variable region IV (VR-IV).

Disclosed herein is an AAV vector comprising an adeno-associated virus (AAV) capsid protein comprising a sequence having at least 95% identity to the sequence set forth in SEQ ID NO:01 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO:02 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO:03 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO:04 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO:05 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO:06 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO:07 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO:08 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO:09 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO:10 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO:11 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO: 12 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO: 13 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO:14 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO: 15 with one or more substitutions in variable region VIII (VR-VIII). Disclosed herein is an AAV vector comprising an adeno-associated virus (AAV) capsid protein comprising a sequence having at least 95% identity to the sequence set forth in SEQ ID NO:16 with one or more substitutions in variable region VIII (VR-VIII).

Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:01. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:02. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:03. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:04. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:05. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:06. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:07. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:08. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:09. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:10. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:11. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:12. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:13. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:14. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO:15. Disclosed herein is an AAV vector comprising an adeno-associated virus (AAV) capsid protein comprising a sequence having one or more substitutions in variable region IV (VR-IV) and variable region VIII (VR-VIII) relative to SEQ ID NO: 16.

In an aspect, a disclosed AAV vector can be used to improve and/or can be used to enhance gene transfer efficiency in intestinal epithelial cells and/or colonic epithelial cells. In an aspect, a disclosed AAV vector can be used to improve and/or can be used to enhance gene transfer efficiency in intestinal epithelial cells and/or colonic epithelial cells. In an aspect, a disclosed AAV vector can be used to improve and/or can be used to enhance gene transfer efficiency in intestinal epithelial cells and/or colonic epithelial cells. In an aspect, a disclosed AAV vector can be used to improve and/or can be used to enhance the quality of the subject's life when compared to a pre-treatment level. In an aspect, a disclosed AAV vector can be used to diminish and/or decrease one or more symptoms associated with and/or related to the subject's diseases and/or disorder. In an aspect, a disclosed AAV vector can be used to prevent an undesired physiological change, disease, pathological condition, or disorder from occurring in the subject. In an aspect, a disclosed AAV vector can be used to inhibit a physiological change, disease, pathological condition, or disorder, i.e., arresting its development, in the subject. In an aspect, a disclosed AAV vector can be used to relieve a physiological change, disease, pathological condition, or disorder, i.e., causing regression of the disease, in the subject. In In an aspect, a disclosed AAV vector can be used to improve the subject's quality of life by at least 50% when compared to the subject's pre-treatment quality of life.

In an aspect, a disclosed AAV vector can be used to improve and/or can be used to alleviate one or more gastrointestinal tract manifestations of cystic fibrosis in the subject. In an aspect, a GI tract manifestation of cystic fibrosis can comprise mucous inspissation, dysmotility, meconium ileus (MI), constipation, distal intestinal obstruction syndrome (DIOS), gastroesophageal reflux disease (GERD), small bowel bacterial overgrowth, or any combination thereof. In an aspect, a disclosed AAV vector can be used to improve and/or can be used to alleviate nutritional failure in the subject. In an aspect, a disclosed AAV vector can be used to improve the malabsorption of fat, protein, and/or fat-soluble vitamins in the subject. In an aspect, a disclosed AAV vector can be used to improve the malabsorption of fat, protein, and/or fat-soluble vitamins in the subject.

In an aspect, a disclosed AAV vector can be used to improve pancreatic insufficiency in the subject. In an aspect, a disclosed AAV vector can be used to decrease the risk for recurrent pancreatitis in the subject. In an aspect, a disclosed AAV vector can be used to reduce the risk of developing chronic lung disease in the subject. In an aspect, a disclosed AAV vector can be used to reduce the risk of developing a pulmonary infection and/or progressive pulmonary infection in the subject. In an aspect, a disclosed AAV vector can be used to repair diseased and/or dysfunctional intestinal epithelium and/or colonic epithelium.

In an aspect, a disclosed AAV vector can be used to reduce intestinal and/or colonic inflammation in the subject. In an aspect, a disclosed AAV vector can be used to treat a subject in need thereof. In an aspect, a disclosed AAV vector can be used in a method of delivering gene therapy to a subject in need thereof.

In an aspect, a disclosed AAV vector can demonstrate improved tropism for one or more cell types and/or one or more tissue types. In an aspect, a disclosed AAV vector can exhibit improved transduction efficiency and/or properties when introduced to one or more cell types and/or one or more tissue types. In an aspect, AAVcc47 can efficiently transduce the cells of the intestinal epithelium and/or colonic epithelium. In an aspect, AAVcc47 can outperform wild-type AAV9 in the cells of the intestinal epithelium and/or colonic epithelium.

In an aspect, a disclosed AAV vector can be AAV1, AAV2, AAV3 (including 3a and 3b), AAV4, AAV5, AAV6. AAV7, AAV8, AAVrh8, AAV9. AAV10, AAVrh10, AAV1I, AAV12, AAV13, AAVrh39, AAVrh43, or AAVcy.7. In an aspect, a disclosed AAV vector can be bovine AAV, caprine AAV, canine AAV, equine AAV, ovine AAV, avian AAV, primate AAV, or non-primate AAV. In an aspect, a disclosed AAV vector can be AAV-DJ, AAV-HAE1. AAV-HAE2, AAVM41, AAV-1829. AAV2 Y/F, AAV2 TN, AAV2i8, AAV2.5. AAV9.45, AAV9.61, AAV-B1. AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, a disclosed AAV vector can comprise one or more regulatory elements. A disclosed vector can comprise a ubiquitous promoter operably linked to a disclosed isolated nucleic acid molecule, wherein the ubiquitous promoter drives the expression of a disclosed transgene or gene of interest, a disclosed encoded polypeptide, a disclosed encoded therapeutic agent, or both. A disclosed vector can comprise a tissue specific promoter operably linked to a disclosed isolated nucleic acid molecule, wherein the tissue specific promoter drives the expression of a disclosed transgene or gene of interest, a disclosed encoded polypeptide, a disclosed encoded therapeutic agent, or both.

In an aspect, a disclosed vector can be formulated for administration via one or more routes. Such routes are well known to those skilled in the art and include, but are not limited to, the following: colonoscopy-guided administration, oral administration, transdermal administration, administration by inhalation, nasal administration, topical administration, intravaginal administration, ophthalmic administration, intraaural administration, otic administration, inter utero administration, intracerebral administration, rectal administration, sublingual administration, buccal administration, and parenteral administration, including injectable such as intravenous administration, intra-arterial administration, intramuscular administration, and subcutaneous administration. Administration of a disclosed vector can be continuous or intermittent.

In an aspect, a disclosed vector can be used in a disclosed method. In an aspect, a disclosed vector can be used in a disclosed method of delivering a payload. In an aspect, a disclosed vector can be used in a disclosed method of treating of subject.

### 6. Pharmaceutical Formulations

Disclosed herein is a pharmaceutical formulation comprising a disclosed AAV in a pharmaceutically acceptable carrier. Disclosed herein is a pharmaceutical formulation comprising a disclosed AAV vector in a pharmaceutically acceptable carrier. Disclosed herein is a pharmaceutical formulation comprising a disclosed nucleic acid molecule in a pharmaceutically acceptable carrier.

In an aspect, a disclosed pharmaceutical formulation can be administered to a subject in need thereof. In an aspect, a disclosed AAV vector can comprise an AAV having a variant capsid. Variants AAV capsid proteins are disclosed herein. In an aspect, a disclosed pharmaceutical formulation can be administered to a subject in need thereof to deliver a payload. In an aspect, a disclosed pharmaceutical formulation can be administered to a subject in need thereof to treat and/or prevent a disease or disorder. Pharmaceutically acceptable carriers are known to the art and are discussed supra.

In an aspect, a disclosed pharmaceutical formulation can be formulated for administration via one or more routes. Such routes are well known to those skilled in the art and include, but are not limited to, the following: colonoscopy-guided administration, oral administration, transdermal administration, administration by inhalation, nasal administration, topical administration, intravaginal administration, ophthalmic administration, intraaural administration, otic administration, inter utero administration, intracerebral administration, rectal administration, sublingual administration, buccal administration, and parenteral administration, including injectable such as intravenous administration, intra-arterial administration, intramuscular administration, and subcutaneous administration. Administration of a disclosed pharmaceutical formulation can be continuous or intermittent.

In an aspect, a disclosed pharmaceutical formulation can be used in a disclosed method. In an aspect, a disclosed pharmaceutical formulation can be used in a disclosed method of delivering a payload.

### 7. Kits

Disclosed herein is a kit comprising one or more disclosed compositions. In an aspect, a composition of a disclosed kit can comprise a disclosed isolated nucleic acid molecule, a disclosed plasmid, a disclosed variant AAV capsid protein, a disclosed AAV comprising a capsid having a variant AAV capsid protein, a disclosed AAV vector, a disclosed pharmaceutical composition, or a combination thereof. In an aspect, a disclosed kit can comprise a combination of one or more active agents. In an aspect, a disclosed kit can comprise at least two components constituting the kit. Together, the components constitute a functional unit for a given purpose (such as, for example, performing directed evolution on AAV capsid proteins, generating AAV particles, or delivering a payload). Individual member components can be physically packaged together or separately. For example, a disclosed kit comprising an instruction for using the kit may or may not physically include the instruction with other individual member components. Instead, the instruction can be supplied as a separate member component, either in a paper form or an electronic form that can be supplied on computer readable memory device or downloaded from an internet website, or as recorded presentation. In an aspect, a disclosed kit for use in a disclosed method can comprise one or more containers holding a disclosed composition (i.e., a disclosed AAV capsid protein, a disclosed AAV, a disclosed vector, a disclosed plasmid, a disclosed primer, a disclosed pharmaceutical formulation, or any combination thereof) and a label or package insert with instructions for use.

In an aspect, a disclosed kit can contain one or more additional agents (e.g., excipients, buffers, active agents, biologically active agents, pharmaceutically active agents, immune-based therapeutic agents, clinically approved agents, or a combination thereof). In an aspect, one or more active agents can treat, inhibit, and/or ameliorate one or more comorbidities in a subject. In an aspect, one or more active agents can treat, inhibit, and/or ameliorate a disease, a disorder, an infection, a symptom, a complication, or a combination thereof. In an aspect, suitable containers include, for example, bottles, vials, syringes, blister pack, etc. The containers can be formed from a variety of materials such as glass or plastic. The container can hold a disclosed composition or a pharmaceutical formulation comprising a disclosed composition and can have a sterile access port (for example the container can be an intravenous solution bag or a vial having a stopper pierceable by a hypodermic injection needle). The label or package insert can indicate that a disclosed composition or a pharmaceutical formulation comprising a disclosed composition can be used for treating, preventing, inhibiting, and/or ameliorating a disease, a disorder, an infection, a symptom, a complication, or a combination thereof. A disclosed kit can comprise additional components necessary for administration such as, for example, other buffers, diluents, filters, needles, and syringes. The term “package insert” can refer to instructions customarily included in commercial packages of therapeutic products, that contain information about the indications, usage, dosage, administration, contraindications and/or warnings concerning the use of such therapeutic products.

In an aspect, a disclosed kit can comprise some or all of the components necessary to practice and/or perform one or more disclosed methods.

### C. Methods

### 1. Methods of Delivering a Payload

Disclosed herein is a method for delivering a payload, the method comprising contacting one or more cells in a subject with a therapeutically effective amount of a disclosed nucleic acid molecule encoding a gene of interest or a transgene; and expressing the encoded gene of interest or transgene.

Disclosed herein is a method for delivering a payload, the method comprising contacting one or more cells in a subject with a therapeutically effective amount of an AAV vector encoding a gene of interest or a transgene; and expressing the encoded gene of interest or transgene.

Disclosed herein is a method for delivering a payload, the method comprising contacting one or more cells in a subject with a therapeutically effective amount of a disclosed AAV vector (i) comprising a disclosed variant capsid protein and (ii) encoding a gene of interest or a transgene; and expressing the encoded gene of interest or transgene.

Disclosed herein is a method of delivering a payload, the method comprising contacting one or more cells with a disclosed nucleic acid molecule or with a disclosed rAAV vector, wherein the contacting allows for expression of the payload in the one or more cells.

In an aspect, a disclosed AAV vector can comprise a disclosed variant AAV capsid proteins. In an aspect, a disclosed variant AAV capsid protein can comprise any variant AAV capsid protein disclosed herein.

In an aspect, a disclosed AAV vector can comprise AAVcc47. In an aspect, a disclosed variant AAV capsid protein can comprise the sequence set forth in any one of SEQ ID NO:58-SEQ ID NO:66. In an aspect, a disclosed variant AAV capsid protein can comprise the sequence set forth in any one of SEQ ID NO:76-SEQ ID NO:84. In an aspect, a disclosed variant AAV capsid protein can comprise the sequence set forth in any one of SEQ ID NO:85-SEQ ID NO:165. In an aspect, a disclosed variant AAV capsid protein can comprise the sequence set forth in any one of SEQ ID NO:172-SEQ ID NO:187. In an aspect, a disclosed variant AAV capsid protein can comprise the sequence set forth in any one of SEQ ID NO: 188-SEQ ID NO:203.

In an aspect, a disclosed payload can refer to a nucleic acid that is encapsidated within a disclosed AAV vector. In an aspect, a disclosed payload nucleic acid can encode a polypeptide, an inhibitory RNA, an antibody or antibody reagent, an oligonucleotide, or a miRNA. In an aspect, a disclosed payload refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid. In an aspect, a disclosed payload can comprise any nucleic acid that is useful for modulating the expression in a target cell transduced or contacted with the AAV particle carrying the payload. In an aspect, modulation can be by supplementation of the payload in a target cell or tissue. In an aspect, modulation can be gene replacement of the payload in a target cell or tissue. In an aspect, modulation can be by inhibition using a modulatory nucleic acid of the payload in a target cell or tissue. In an aspect, a disclosed payload can comprise a combination of coding and non-coding nucleic acid sequences, and can be codon-optimized. In an aspect, a disclosed payload can comprise one or more regulatable elements. In an aspect, a disclosed payload can encode a messenger RNA (mRNA) can be encoded by a disclosed payload. In an aspect, a disclosed payload can encode a gene therapy product. A gene therapy product can comprise a polypeptide, RNA molecule, or other gene product that, when expressed in a target cell, provides a desired therapeutic effect. In an aspect, a gene therapy product can comprise a substitute for a non-functional gene that is absent or mutated. In an aspect, a disclosed payload nucleic acid can encode a transgene having a beneficial or desirable gene product (such as, for example, CFTR). In an aspect, a disclosed payload can encode one or more a base-editing components and/or one or more gRNA targeting the region to be edited.

In an aspect, a subject can have cystic fibrosis. In an aspect, a subject can have any genetic disease or disorder that affects the gastrointestinal system. In an aspect, a subject can have colitis (e.g., infections colitis, ulcerative colitis, Crohn's disease, ischemic colitis, or any combination thereof). In an aspect, a subject can have peptic cystic fibrosis, ulcer disease, gastritis, gastroenteritis, celiac disease, Crohn's disease, gallstones, fecal incontinence, lactose intolerance, Hirschsprung disease, abdominal adhesions, Barrett's esophagus, appendicitis, indigestion (dyspepsia), intestinal pseudo-obstruction, pancreatitis, short bowel syndrome, Whipple's disease, Zollinger-Ellison syndrome, malabsorption syndromes, hepatitis, or any combination thereof.

In an aspect of a disclosed method, a subject can be treatment-naïve. In an aspect, a subject can have received prior treatment.

In an aspect, a therapeutically effective amount of disclosed vector can be delivered via intravenous (IV) administration or colonoscopy-guided administration and can comprise a range of about 1×10¹⁰ vg/kg to about 2×10¹⁴vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a therapeutically effective amount of disclosed vector can be delivered via intravenous (IV) administration or colonoscopy-guided administration and can comprise a range of about 1×10¹² vg per subject total to about 1×10¹⁷vg per subject total. In an aspect, a therapeutically effective amount of disclosed vector can be delivered via intravenous (IV) administration and can comprise a range of about 1×10¹² vg per subject total, about 1×10¹³vg per subject total, about 1×10¹⁴ vg per subject total, about 1×10¹⁵ vg per subject total, about 1×10¹⁶ vg per subject total, or about 1×10¹⁷ vg per subject total. In an aspect, a disclosed vector can be administered in a single dose, or in multiple doses (such as 2, 3, 4, 5, 6, 7, 8, 9 or 10 doses) as needed for the desired therapeutic results. In an aspect, a therapeutically effective amount of disclosed vector can comprise a range determined by a skilled person. In an aspect, a therapeutically effective amount of disclosed vector can be delivered via intravenous (IV) administration and can comprise a range of about 1×10¹² vg per subject total to about 1×10¹⁷ vg per subject total. In an aspect, a therapeutically effective amount of disclosed vector can be delivered via intravenous (IV) administration or colonoscopy-guided administration and can comprise a range of about 1×10¹² vg per subject total, about 1×10¹³vg per subject total, about 1×10¹⁴ vg per subject total, about 1×10¹⁵ vg per subject total, about 1×10¹⁶ vg per subject total, or about 1×10¹⁷ vg per subject total. In an aspect, a therapeutically effective amount of disclosed vector can be delivered by colonoscopy-guided administration and can comprise a range of about 1×10¹² vg per subject total to about 1×10¹⁷ vg per subject total. In an aspect, a therapeutically effective amount of disclosed vector can be delivered by colonoscopy-guided administration and can comprise a range of about 1×10¹² vg per subject total, about 1×10¹³vg per subject total, about 1×10¹⁴ vg per subject total, about 1×10¹⁵vg per subject total, about 1×10¹⁶ vg per subject total, or about 1×10¹⁷vg per subject total.

In an aspect, a disclosed method can be used to improve and/or can be used to enhance gene transfer efficiency in intestinal epithelial cells and/or colonic epithelial cells. In an aspect, a disclosed method can be used to improve and/or can be used to enhance gene transfer efficiency in intestinal epithelial cells and/or colonic epithelial cells. In an aspect, a disclosed method can be used to improve and/or can be used to enhance gene transfer efficiency in intestinal epithelial cells and/or colonic epithelial cells. In an aspect, a disclosed method can be used to improve and/or can be used to enhance the quality of the subject's life when compared to a pre-treatment level. In an aspect, a disclosed method can be used to diminish and/or decrease one or more symptoms associated with and/or related to the subject's diseases and/or disorder. In an aspect, a disclosed method can be used to prevent an undesired physiological change, disease, pathological condition, or disorder from occurring in the subject. In an aspect, a disclosed method can be used to inhibit a physiological change, disease, pathological condition, or disorder, i.e., arresting its development, in the subject. In an aspect, a disclosed method can be used to relieve a physiological change, disease, pathological condition, or disorder, i.e., causing regression of the disease, in the subject. In an aspect, a disclosed method can be used to improve the subject's quality of life by at least 50% when compared to the subject's pre-treatment quality of life.

In an aspect, a disclosed method can be used to improve and/or can be used to alleviate one or more gastrointestinal tract manifestations of cystic fibrosis in the subject. In an aspect, a GI tract manifestation of cystic fibrosis can comprise mucous inspissation, dysmotility, meconium ileus (MI), constipation, distal intestinal obstruction syndrome (DIOS), gastroesophageal reflux disease (GERD), small bowel bacterial overgrowth, or any combination thereof. In an aspect, a disclosed method can be used to improve and/or can be used to alleviate nutritional failure in the subject. In an aspect, a disclosed method can be used to improve the malabsorption of fat, protein, and/or fat-soluble vitamins in the subject. In an aspect, a disclosed method can be used to improve the malabsorption of fat, protein, and/or fat-soluble vitamins in the subject. In an aspect, a disclosed method can be used to improve pancreatic insufficiency in the subject. In an aspect, a disclosed method can be used to decrease the risk for recurrent pancreatitis in the subject.

In an aspect, a disclosed method can be used to reduce the risk of developing chronic lung disease in the subject. In an aspect, a disclosed method can be used to reduce the risk of developing a pulmonary infection and/or progressive pulmonary infection in the subject.

In an aspect, a disclosed method can be used to repair diseased and/or dysfunctional intestinal epithelium and/or colonic epithelium. In an aspect, a disclosed method can be used to reduce intestinal and/or colonic inflammation in the subject.

In an aspect, a disclosed method can be used to treat a subject in need thereof. In an aspect, a disclosed method can be used in a method of delivering gene therapy to a subject in need thereof.

In an aspect of a disclosed method, restoring the activity and/or functionality of a missing, deficient, and/or mutant protein or enzyme (e.g., CFTR) can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme (e.g., CFTR). In an aspect, restoration can be a partial or incomplete restoration. In an aspect, restoration can be complete or near complete restoration such that the level of expression, activity, and/or functionality (for CFTR, for example) is similar to that of a wild-type or control level.

In an aspect of a disclosed method, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect of a disclosed method, contacting a cell can comprising methods known to the art. For example, contacting can comprise administering to a subject one or more disclosed enzymes or recombinant enzymes, one or more of the disclosed isolated nucleic acid molecules, one or more disclosed AAV vectors, one or more disclosed pharmaceutical formulations, or any combination thereof.

In an aspect, administering can comprise colongoscopy-guided, intravenous, intraarterial, intramuscular, intraperitoneal, subcutaneous, intra-CSF, intrathecal, intraventricular, intrahepatic, hepatic intra-arterial, hepatic portal vein (HPV), or in utero administration. In an aspect, a disclosed composition, a disclosed enzyme or disclosed recombinant enzyme, a disclosed isolated nucleic acid molecule, a disclosed pharmaceutical formulation, and/or a disclosed AAV vector can be administered via colonoscopy-guided administration in combination with RNAi, antisense oligonucleotides, miRNA, one or more small molecules, one or more therapeutic agents, one or more proteasome inhibitors, one or more replacement enzymes, one or more immune modulators, and/or a gene editing system. In an aspect, a disclosed composition, a disclosed enzyme or disclosed recombinant enzyme, a disclosed isolated nucleic acid molecule, a disclosed pharmaceutical formulation, and/or a disclosed AAV vector can be administered via LNP administration. In an aspect, a disclosed composition, a disclosed isolated nucleic acid molecule, a disclosed pharmaceutical formulation, and/or a disclosed AAV vector can be concurrently and/or serially administered to a subject via multiple routes of administration. For example, in an aspect, administering a disclosed composition, a disclosed enzyme or disclosed recombinant enzyme, a disclosed isolated nucleic acid molecule, a disclosed pharmaceutical formulation, and/or a disclosed AAV vector can comprise IV administration and colonoscopy-guided administration. In an aspect, a disclosed method can employ multiple routes of administration to the subject. In an aspect, a disclosed method can employ a first route of administration that can be the same or different as a second and/or subsequent routes of administration.

In an aspect, colonoscopy-guided administration can comprise creating a bubble under the mucosa. In an aspect, a disclosed AAV vector can be injected by colonoscopy-guided injection into the submucosa of the organ.

In an aspect, a therapeutically effective amount of a disclosed replacement enzyme or disclosed recombinant enzyme can comprise about 0.01 mg/kg body weight to about 100 mg/kg body weight. In an aspect, a disclosed enzyme or disclosed recombinant enzyme can be therapeutically effective when the dose comprises about 0.01 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg body weight.

In an aspect of a disclosed method of delivering a payload, a therapeutically effective amount of disclosed AAV vector can be delivered to a subject via intravenous (IV) administration and/or colonoscopy-guided administration and can comprise a range of about 1×10¹⁰ vg/kg to about 2×10¹⁴vg/kg. In an aspect, for example, a disclosed AAV vector can be administered at a dose of about 1×10¹¹ vg/kg to about 8×10¹³ vg/kg or about 1×10¹² vg/kg to about 8×10¹³ vg/kg. In an aspect, a disclosed AAV vector can be administered at a dose of about 1×10¹³ vg/kg to about 6×10¹³ vg/kg. In an aspect, a disclosed AAV vector can be administered at a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, a disclosed AAV vector can be administered at a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³, or no more than about 1×10¹⁴ vg/kg. In an aspect, a disclosed AAV vector can be administered to a subject at a dose of about 1×10¹² vg/kg. In an aspect, a disclosed AAV vector can be administered to a subject at a dose of about 1×10¹¹ vg/kg. In an aspect, a therapeutically effective amount of disclosed vector can be delivered via intravenous (IV) administration and can comprise a range of about 1×10¹² vg per subject total to about 1×10¹⁷ vg per subject total. In an aspect, a therapeutically effective amount of disclosed AAV vector can be delivered via intravenous (IV) administration and/or colonoscopy-guided administration and can comprise a range of about 1×10¹² vg per subject total, about 1×10¹³vg per subject total, about 1×10¹⁴ vg per subject total, about 1×10¹⁵vg per subject total, about 1×10¹⁶ vg per subject total, or about 1×10¹⁷vg per subject total. In an aspect, a disclosed AAV vector can be administered in a single dose, or in multiple doses (such as 2, 3, 4, 5, 6, 7, 8, 9 or 10 doses) as needed for the desired therapeutic results.

In an aspect, a disclosed method of delivering a payload can further comprise administering to the subject a therapeutically effective amount of a therapeutic agent. A therapeutic agent can be any disclosed agent that effects a desired clinical outcome.

In an aspect of a disclosed method delivering a payload, the administering step can treat a subject in need thereof. In an aspect, treating a subject can comprising administering one or more times to the subject one or more additional therapies. In an aspect, a disclosed method of delivering a payload can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method of delivering a payload can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway. For example, in an aspect, a disclosed method can comprise administering to the subject one or more times pancreatic enzyme replacement therapy (PERT). In an aspect, a diose of PERT can comprise about 500 to about 2500 units lipase per kg body weight per meal or about less than 10,000 units lipase per kg body weight per day.

In an aspect, a disclosed method of delivering a payload can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed AAV vector. In an aspect, a disclosed immune modulator can be administered subcutaneously about 15 minutes before a disclosed AAV vector. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed AAV vector, a disclosed pharmaceutical formulation, or a combination thereof. In an aspect, a disclosed immune modulator can be administered subcutaneously about 15 minutes before or a few days before a disclosed isolated nucleic acid molecule, a disclosed AAV vector, a disclosed pharmaceutical formulation, or a combination thereof. In an aspect, a disclosed immune modulator can be administered concurrently with a disclosed isolated nucleic acid molecule, a disclosed AAV vector, a disclosed pharmaceutical formulation, or a combination thereof.

In an aspect, a disclosed method of improving and/or enhancing transgene efficacy and/or expression can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a method of delivering a payload can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method can further comprise administering lipid nanoparticles (LNPs). In an aspect, LNPs can be organ-targeted (such as, for example, the intestinal epithelium and/or the colonic epithelium). In an aspect, LNPs can be liver-targeted or testes-targeted. For example, in an aspect, mRNA therapy with LNP encapsulation for systemic delivery to a subject has the potential to restore the functionality and/or structural integrity of a missing, deficient, and/or mutant protein or enzyme.

In an aspect, a disclosed method of delivering a payload can further comprise plasmapheresis and immunosuppression. In an aspect, a disclosed method can comprise using immunosuppression to decrease the T cell, B cell, and/or plasma cell population, decrease the innate immune response, inflammatory response, and antibody levels in general.

In an aspect, a disclosed method can comprise repeating a disclosed administering step such as, for example, repeating the administering of a disclosed enzyme, a disclosed recombinant enzyme (e.g., PERT), a disclosed isolated nucleic acid molecule, a disclosed AAV vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method of delivering a payload can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more disclosed enzymes or recombinant enzymes (e.g., PERT), one or more of the disclosed isolated nucleic acid molecules, one or more disclosed AAV vectors, one or more disclosed pharmaceutical formulations, or any combination thereof administered to a subject, or by changing the frequency of administration of one or more disclosed enzymes or recombinant enzymes, one or more of the disclosed isolated nucleic acid molecules, one or more disclosed AAV vectors, one or more disclosed pharmaceutical formulations, or any combination thereof to a subject, or by changing the duration of time one or more disclosed enzymes or recombinant enzymes, one or more of the disclosed isolated nucleic acid molecules, one or more disclosed AAV vectors, one or more disclosed pharmaceutical formulations, or any combination thereof are administered to a subject.

In an aspect, a disclosed method can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In as aspect, a disclosed method can comprise concurrent administration of one or more of the following: one or more disclosed enzymes or recombinant enzymes (e.g., PERT), one or more of the disclosed isolated nucleic acid molecules, one or more disclosed AAV vectors, one or more disclosed pharmaceutical formulations, one or more disclosed therapeutic agents, one or more disclosed immune modulators, one or more disclosed proteasome inhibitors, one or more disclosed immunosuppressive agents, one or more disclosed compounds that exert therapeutic effect against B cells, one or more disclosed compounds that targets or alters antigen presentation or humoral or cell mediated immune response, or any combination thereof.

In an aspect, a disclosed immune modulator can be administered prior to or after the administration of a disclosed therapeutic agent. In an aspect, a disclosed method of delivering a payload can further comprise generating one or more disclosed enzymes or disclosed recombinant enzymes, one or more of the disclosed isolated nucleic acid molecules, one or more disclosed AAV vectors, one or more disclosed pharmaceutical formulations, or any combination thereof.

For example, in an aspect, a disclosed method of delivering a payload can further comprise generating a disclosed AAV viral or non-viral vector. In an aspect, generating a disclosed viral vector can comprise generating an AAV vector or a recombinant AAV (such as those disclosed herein).

In an aspect, a disclosed method of delivering a payload can further comprise gene editing one or more relevant genes (such as, for example, a missing, deficient, and/or mutant protein or enzyme), wherein editing includes but is not limited to single gene knockout, loss of function screening of multiple genes at one, gene knockin, or a combination thereof.

In an aspect of a disclosed method, a payload can comprise one or more base-editing components and one or more sgRNA targeting the region to be edited. In an aspect, a disclosed region to be edited can comprise a W1282X CFTR mutation and/or a R1283G mutation. In an aspect, a disclosed base-editing component can comprise an adenine base editor.

In an aspect, a disclosed method of delivering a payload can further comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. In an aspect, a disclosed Cas9 can be from Staphylococcus aureus or Streptococcus pyogenes.

In an aspect, a disclosed method of delivering a payload can further comprise generating and/or validating one or more disclosed enzymes or disclosed recombinant enzymes, one or more of the disclosed isolated nucleic acid molecules, one or more disclosed AAV vectors, one or more disclosed pharmaceutical formulations, or any combination thereof.

In an aspect of a disclosed method, a disclosed enzyme, a disclosed recombinant enzyme, a disclosed isolated nucleic acid molecule, a disclosed AAV vector, a disclosed pharmaceutical formulation, or any combination thereof can be delivered and/or administered prior to, concurrent, or after the delivery and/or administration of enzyme replacement therapy, protein replacement, gene therapy, a recombinant product, or any combination thereof.

In an aspect, a disclosed method of delivering a payload can further comprise reducing and/or minimizing vector-mediated immunotoxicity and/or transgene immunogenicity (e.g., the ability to induce specific immunity). In an aspect, vector-mediated immunotoxicity and/or transgene immunogenicity can decrease and/or reduce the efficacy of the recombinant product encoded by the transgene. In an aspect, vector-mediated immunotoxicity and/or transgene immunogenicity can decrease and/or reduce the ability and/or likelihood of re-dosing a subject with one or more disclosed enzymes or recombinant enzymes, one or more of the disclosed isolated nucleic acid molecules, one or more disclosed AAV vectors, one or more disclosed pharmaceutical formulations, or any combination thereof. In an aspect, vector-mediated immunotoxicity and/or transgene immunogenicity can decrease and/or reduce the ability and/or likelihood of re-dosing a subject with gene therapy, enzyme replacement therapy, protein replacement, or any combination thereof.

In an aspect, a disclosed method can further comprise administering one or more times one or more disclosed enzymes or recombinant enzymes, one or more of the disclosed isolated nucleic acid molecules, one or more disclosed AAV vectors, one or more disclosed pharmaceutical formulations, or any combination thereof.

In an aspect, a disclosed method can further comprise measuring and/or determining a subject's pre-treatment level of one or more clinical and/or metabolic indicators (such as, for example, the expression of CFTR). In an aspect, a disclosed method can further comprise measuring and/or determining one or more times a subject's level of one or more clinical and/or metabolic indicators (such as CFTR).

2. Methods of Generating and/or Making AAV Particles

Disclosed herein is a method of generating AAV particles, the method comprising delivering to one or more cells a nucleic acid molecule comprising a nucleic acid sequence encoding a variant AAV capsid protein; culturing the one or more cells; and harvesting the AAV particles from the one or more producer cells. Disclosed herein is a method of generating AAV particles, the method comprising delivering to one or more cells three plasmids, wherein the first plasmid is a helper plasmid, wherein the second plasmid is RepCap plasmid, wherein the second plasmid comprises a nucleic acid molecule comprising a nucleic acid sequence encoding a variant AAV capsid protein, and wherein the third plasmid is the cis-plasmid or transfer plasmid; culturing the one or more cells; and harvesting the AAV particles from the one or more cells. Disclosed herein is a method for making an AAV particle, the method comprising providing one or more cells comprising a disclosed AAV vector and culturing the one or more cells under conditions allowing for forming an AAV particle.

In an aspect, a disclosed method can comprise expressing a disclosed encoded variant AAV capsid protein. In an aspect, a disclosed method can comprise culturing the one or more cells in a media. In an aspect, a disclosed method can comprise harvesting the AAV particles. In an aspect, a disclosed method can comprise purifying the harvested AAV particles. In an aspect, a disclosed method can comprise using the purified AAV particles in gene therapy. In an aspect, a disclosed method can comprise delivering to the one or more cells a helper plasmid. In an aspect, a disclosed method can comprise delivering to the one or more cells a cis-plasmid or a transfer plasmid encoding a gene of interest or a transgene. In an aspect, disclosed secreted AAV particles can comprise the gene of interest or the transgene. In an aspect, disclosed secreted AAV particles can comprise one or more base-editing components and/or one or more gRNAs.

In an aspect, a disclosed method of generating and/or making AAV particles can comprise validating the purity of the AAV particles and/or the functionality of the AAV particles.

### 3. Methods of Treating a Subject

Disclosed herein is a method of treating a subject, the method comprising administering one or more times to a subject in need thereof a therapeutically effective amount of a disclosed AAV vector or a disclosed pharmaceutical composition. Disclosed herein is a method for treating a subject, the method comprising contacting one or more cells in a subject with a therapeutically effective amount of a disclosed AAV vector encoding a payload, a gene of interest or a transgene; and expressing the encoded payload, the encoded gene of interest, or the encoded transgene.

Disclosed herein is a method for treating a subject, the method comprising contacting one or more cells in a subject with a therapeutically effective amount of a disclosed AAV vector (i) comprising a disclosed variant capsid protein and (ii) encoding a payload, a gene of interest, or a transgene; and expressing the encoded payload, the encoded gene of interest, or the encoded transgene.

Disclosed herein is a method of treating a subject, the method comprising contacting one or more cells in the subject with a disclosed nucleic acid molecule or with a disclosed rAAV vector, wherein the contacting allows for expression of the encoded payload, the encoded gene of interest, or the encoded transgene in the one or more cells.

In an aspect, a disclosed AAV vector can comprise a disclosed variant AAV capsid proteins. In an aspect, a disclosed variant AAV capsid protein can comprise any variant AAV capsid protein disclosed herein.

In an aspect, a disclosed AAV vector can comprise AAVcc47. In an aspect, a disclosed variant AAV capsid protein can comprise the sequence set forth in any one of SEQ ID NO:58-SEQ ID NO:66. In an aspect, a disclosed variant AAV capsid protein can comprise the sequence set forth in any one of SEQ ID NO:76-SEQ ID NO:84. In an aspect, a disclosed variant AAV capsid protein can comprise the sequence set forth in any one of SEQ ID NO:85-SEQ ID NO: 165. In an aspect, a disclosed variant AAV capsid protein can comprise the sequence set forth in any one of SEQ ID NO:172-SEQ ID NO:187. In an aspect, a disclosed variant AAV capsid protein can comprise the sequence set forth in any one of SEQ ID NO:188-SEQ ID NO:203.

In an aspect, a disclosed payload can refer to a nucleic acid that is encapsidated within a disclosed AAV vector. In an aspect, a disclosed payload nucleic acid can encode a polypeptide, an inhibitory RNA, an antibody or antibody reagent, an oligonucleotide, or a miRNA. In an aspect, a disclosed payload refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid. In an aspect, a disclosed payload can comprise any nucleic acid that is useful for modulating the expression in a target cell transduced or contacted with the AAV particle carrying the payload. In an aspect, modulation can be by supplementation of the payload in a target cell or tissue. In an aspect, modulation can be gene replacement of the payload in a target cell or tissue. In an aspect, modulation can be by inhibition using a modulatory nucleic acid of the payload in a target cell or tissue. In an aspect, a disclosed payload can comprise a combination of coding and non-coding nucleic acid sequences, and can be codon-optimized. In an aspect, a disclosed payload can comprise one or more regulatable elements. In an aspect, a disclosed payload can encode a messenger RNA (mRNA) can be encoded by a disclosed payload. In an aspect, a disclosed payload can encode a gene therapy product. A gene therapy product can comprise a polypeptide, RNA molecule, or other gene product that, when expressed in a target cell, provides a desired therapeutic effect. In an aspect, a gene therapy product can comprise a substitute for anon-functional gene that is absent or mutated. In an aspect, a disclosed payload nucleic acid can encode a transgene having a beneficial or desirable gene product (such as, for example, CFTR). In an aspect, a disclosed payload can encode one or more a base-editing components and/or one or more gRNA targeting the region to be edited.

In an aspect, a subject can have cystic fibrosis. In an aspect, a subject can have any genetic disease or disorder that affects the gastrointestinal system. In an aspect, a subject can have colitis (e.g., infections colitis, ulcerative colitis, Crohn's disease, ischemic colitis, or any combination thereof). In an aspect, a subject can have peptic cystic fibrosis, ulcer disease, gastritis, gastroenteritis, celiac disease, Crohn's disease, gallstones, fecal incontinence, lactose intolerance, Hirschsprung disease, abdominal adhesions, Barrett's esophagus, appendicitis, indigestion (dyspepsia), intestinal pseudo-obstruction, pancreatitis, short bowel syndrome, Whipple's disease, Zollinger-Ellison syndrome, malabsorption syndromes, hepatitis, or any combination thereof.

In an aspect of a disclosed method, a subject can be treatment-naïve. In an aspect, a subject can have received prior treatment.

In an aspect, a therapeutically effective amount of disclosed vector can be delivered via intravenous (IV) administration or colonoscopy-guided administration and can comprise a range of about 1×10¹⁰ vg/kg to about 2×10¹⁴ vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹ at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰ no more than about 5×10¹⁰, no more than about 1×10¹¹ no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a therapeutically effective amount of disclosed vector can be delivered via intravenous (IV) administration or colonoscopy-guided administration and can comprise a range of about 1×10¹² vg per subject total to about 1×10¹⁷vg per subject total. In an aspect, a therapeutically effective amount of disclosed vector can be delivered via intravenous (IV) administration or colonoscopy-guided administration and can comprise a range of about 1×10¹² vg per subject total, about 1×10¹³vg per subject total, about 1×10¹⁴ vg per subject total, about 1×10¹⁵vg per subject total, about 1×10¹⁶ vg per subject total, or about 1×10¹⁷ vg per subject total. In an aspect, a disclosed vector can be administered in a single dose, or in multiple doses (such as 2, 3, 4, 5, 6, 7, 8, 9 or 10 doses) as needed for the desired therapeutic results. In an aspect, a therapeutically effective amount of disclosed vector can comprise a range determined by a skilled person. In an aspect, a therapeutically effective amount of disclosed vector can be delivered via intravenous (IV) administration or colonoscopy-guided administration and can comprise a range of about 1×10¹² vg per subject total to about 1×10¹⁷ vg per subject total. In an aspect, a therapeutically effective amount of disclosed vector can be delivered via intravenous (IV) administration or colonoscopy-guided administration and can comprise a range of about 1×10¹² vg per subject total, about 1×10¹³ vg per subject total, about 1×10¹⁴ vg per subject total, about 1×10¹⁵ vg per subject total, about 1×10¹⁶ vg per subject total, or about 1×10¹⁷vg per subject total. In an aspect, a therapeutically effective amount of disclosed vector can be delivered by colonoscopy-guided administration and can comprise a range of about 1×10¹² vg per subject total to about 1×10¹⁷vg per subject total. In an aspect, a therapeutically effective amount of disclosed vector can be delivered by colonoscopy-guided administration and can comprise a range of about 1×10¹² vg per subject total, about 1×10¹³vg per subject total, about 1×10¹⁴ vg per subject total, about 1×10¹⁵vg per subject total, about 1×10¹⁶ vg per subject total, or about 1×10¹⁷vg per subject total.

In an aspect, a disclosed method of treating a subject can be used to improve and/or can be used to enhance gene transfer efficiency in intestinal epithelial cells and/or colonic epithelial cells. In an aspect, a disclosed method of treating a subject can be used to improve and/or can be used to enhance gene transfer efficiency in intestinal epithelial cells and/or colonic epithelial cells. In an aspect, a disclosed method of treating a subject can be used to improve and/or can be used to enhance gene transfer efficiency in intestinal epithelial cells and/or colonic epithelial cells. In an aspect, a disclosed method of treating a subject can be used to improve and/or can be used to enhance the quality of the subject's life when compared to a pre-treatment level. In an aspect, a disclosed method of treating a subject can be used to diminish and/or decrease one or more symptoms associated with and/or related to the subject's diseases and/or disorder. In an aspect, a disclosed method of treating a subject can be used to prevent an undesired physiological change, disease, pathological condition, or disorder from occurring in the subject. In an aspect, a disclosed method of treating a subject can be used to inhibit a physiological change, disease, pathological condition, or disorder, i.e., arresting its development, in the subject. In an aspect, a disclosed method of treating a subject can be used to relieve a physiological change, disease, pathological condition, or disorder, i.e., causing regression of the disease, in the subject. In an aspect, a disclosed method of treating a subject can be used to improve the subject's quality of life by at least 50% when compared to the subject's pre-treatment quality of life.

In an aspect, a disclosed method of treating a subject can be used to improve and/or can be used to alleviate one or more gastrointestinal tract manifestations of cystic fibrosis in the subject. In an aspect, a GI tract manifestation of cystic fibrosis can comprise mucous inspissation, dysmotility, meconium ileus (MI), constipation, distal intestinal obstruction syndrome (DIOS), gastroesophageal reflux disease (GERD), small bowel bacterial overgrowth, or any combination thereof.

In an aspect, a disclosed method of treating a subject can be used to improve and/or can be used to alleviate nutritional failure in the subject. In an aspect, a disclosed method of treating a subject can be used to improve the malabsorption of fat, protein, and/or fat-soluble vitamins in the subject. In an aspect, a disclosed method of treating a subject can be used to improve the malabsorption of fat, protein, and/or fat-soluble vitamins in the subject. In an aspect, a disclosed method of treating a subject can be used to improve pancreatic insufficiency in the subject. In an aspect, a disclosed method of treating a subject can be used to decrease the risk for recurrent pancreatitis in the subject. In an aspect, a disclosed method of treating a subject can be used to reduce the risk of developing chronic lung disease in the subject. In an aspect, a disclosed method of treating a subject can be used to reduce the risk of developing a pulmonary infection and/or progressive pulmonary infection in the subject. In an aspect, a disclosed method of treating a subject can be used to repair diseased and/or dysfunctional intestinal epithelium and/or colonic epithelium. In an aspect, a disclosed method of treating a subject can be used to reduce intestinal and/or colonic inflammation in the subject. In an aspect, a disclosed method of treating a subject can be used to treat a subject in need thereof. In an aspect, a disclosed method of treating a subject can be used in a method of delivering gene therapy to a subject in need thereof.

In an aspect of a disclosed method, restoring the activity and/or functionality of a missing, deficient, and/or mutant protein or enzyme (e.g., CFTR) can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme (e.g., CFTR). In an aspect, restoration can be a partial or incomplete restoration. In an aspect, restoration can be complete or near complete restoration such that the level of expression, activity, and/or functionality (for CFTR, for example) is similar to that of a wild-type or control level.

In an aspect of a disclosed method of a treating a subject, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect of a disclosed method, contacting a cell can comprising methods known to the art. For example, contacting can comprise administering to a subject one or more disclosed enzymes or recombinant enzymes, one or more of the disclosed isolated nucleic acid molecules, one or more disclosed AAV vectors, one or more disclosed pharmaceutical formulations, or any combination thereof.

In an aspect, administering can comprise colonoscopy-guided, intravenous, intraarterial, intramuscular, intraperitoneal, subcutaneous, intra-CSF, intrathecal, intraventricular, intrahepatic, hepatic intra-arterial, hepatic portal vein (HPV), or in utero administration. In an aspect, a disclosed composition, a disclosed enzyme or disclosed recombinant enzyme, a disclosed isolated nucleic acid molecule, a disclosed pharmaceutical formulation, and/or a disclosed AAV vector can be administered via colonoscopy-guided administration in combination with RNAi, antisense oligonucleotides, miRNA, one or more small molecules, one or more therapeutic agents, one or more proteasome inhibitors, one or more replacement enzymes, one or more immune modulators, and/or a gene editing system. In an aspect, a disclosed composition, a disclosed enzyme or disclosed recombinant enzyme, a disclosed isolated nucleic acid molecule, a disclosed pharmaceutical formulation, and/or a disclosed AAV vector can be administered via LNP administration. In an aspect, a disclosed composition, a disclosed isolated nucleic acid molecule, a disclosed pharmaceutical formulation, and/or a disclosed AAV vector can be concurrently and/or serially administered to a subject via multiple routes of administration. For example, in an aspect, administering a disclosed composition, a disclosed enzyme or disclosed recombinant enzyme, a disclosed isolated nucleic acid molecule, a disclosed pharmaceutical formulation, and/or a disclosed AAV vector can comprise IV administration and colonoscopy-guided administration. In an aspect, a disclosed method can employ multiple routes of administration to the subject. In an aspect, a disclosed method can employ a first route of administration that can be the same or different as a second and/or subsequent routes of administration.

In an aspect, colonoscopy-guided administration can comprise creating a bubble under the mucosa. In an aspect, a disclosed AAV vector can be injected by colonoscopy-guided injection into the submucosa of the organ.

In an aspect, a therapeutically effective amount of a disclosed replacement enzyme or disclosed recombinant enzyme can comprise about 0.01 mg/kg body weight to about 100 mg/kg body weight. In an aspect, a disclosed enzyme or disclosed recombinant enzyme can be therapeutically effective when the dose comprises about 0.01 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg body weight.

In an aspect, a disclosed method of treating a subject can further comprise administering to the subject a therapeutically effective amount of a therapeutic agent. A therapeutic agent can be any disclosed agent that effects a desired clinical outcome.

In an aspect of a disclosed method of a treating a subject, the administering step can treat a subject in need thereof. In an aspect, treating a subject can comprising administering one or more times to the subject one or more additional therapies. In an aspect, a disclosed method of delivering a payload can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method of treating a subject can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway. For example, in an aspect, a disclosed method can comprise administering to the subject one or more times pancreatic enzyme replacement therapy (PERT). In an aspect, a diose of PERT can comprise about 500 to about 2500 units lipase per kg body weight per meal or about less than 10,000 units lipase per kg body weight per day.

In an aspect, a disclosed method of treating a subject can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed AAV vector. In an aspect, a disclosed immune modulator can be administered subcutaneously about 15 minutes before a disclosed AAV vector. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed AAV vector, a disclosed pharmaceutical formulation, or a combination thereof. In an aspect, a disclosed immune modulator can be administered subcutaneously about 15 minutes before or a few days before a disclosed isolated nucleic acid molecule, a disclosed AAV vector, a disclosed pharmaceutical formulation, or a combination thereof. In an aspect, a disclosed immune modulator can be administered concurrently with a disclosed isolated nucleic acid molecule, a disclosed AAV vector, a disclosed pharmaceutical formulation, or a combination thereof.

In an aspect of a disclosed method of a treating a subject can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect of a disclosed method of a treating a subject can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method can further comprise administering lipid nanoparticles (LNPs). In an aspect, LNPs can be organ-targeted (such as, for example, the intestinal epithelium and/or the colonic epithelium). In an aspect, LNPs can be liver-targeted or testes-targeted. For example, in an aspect, mRNA therapy with LNP encapsulation for systemic delivery to a subject has the potential to restore the functionality and/or structural integrity of a missing, deficient, and/or mutant protein or enzyme.

In an aspect, a disclosed method of delivering a payload can further comprise plasmapheresis and immunosuppression. In an aspect, a disclosed method can comprise using immunosuppression to decrease the T cell, B cell, and/or plasma cell population, decrease the innate immune response, inflammatory response, and antibody levels in general.

In an aspect, a disclosed method can comprise repeating a disclosed administering step such as, for example, repeating the administering of a disclosed enzyme, a disclosed recombinant enzyme (e.g., PERT), a disclosed isolated nucleic acid molecule, a disclosed AAV vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method of treating a subject can comprise modifying one or more of the disclosed steps. For example, modifying one or more of steps of a disclosed method can comprise modifying or changing one or more features or aspects of one or more steps of a disclosed method. For example, in an aspect, a disclosed method can be altered by changing the amount of one or more disclosed enzymes or recombinant enzymes (e.g., PERT), one or more of the disclosed isolated nucleic acid molecules, one or more disclosed AAV vectors, one or more disclosed pharmaceutical formulations, or any combination thereof administered to a subject, or by changing the frequency of administration of one or more disclosed enzymes or recombinant enzymes, one or more of the disclosed isolated nucleic acid molecules, one or more disclosed AAV vectors, one or more disclosed pharmaceutical formulations, or any combination thereof to a subject, or by changing the duration of time one or more disclosed enzymes or recombinant enzymes, one or more of the disclosed isolated nucleic acid molecules, one or more disclosed AAV vectors, one or more disclosed pharmaceutical formulations, or any combination thereof are administered to a subject.

In an aspect, a disclosed method of treating a subject can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In an aspect, a disclosed method of treating a subject can comprise concurrent administration of one or more of the following: one or more disclosed enzymes or recombinant enzymes (e.g., PERT), one or more of the disclosed isolated nucleic acid molecules, one or more disclosed AAV vectors, one or more disclosed pharmaceutical formulations, one or more disclosed therapeutic agents, one or more disclosed immune modulators, one or more disclosed proteasome inhibitors, one or more disclosed immunosuppressive agents, one or more disclosed compounds that exert therapeutic effect against B cells, one or more disclosed compounds that targets or alters antigen presentation or humoral or cell mediated immune response, or any combination thereof.

In an aspect, a disclosed immune modulator can be administered prior to or after the administration of a disclosed therapeutic agent.

In an aspect, a disclosed method of treating a subject can further comprise generating one or more disclosed enzymes or disclosed recombinant enzymes, one or more of the disclosed isolated nucleic acid molecules, one or more disclosed AAV vectors, one or more disclosed pharmaceutical formulations, or any combination thereof.

In an aspect, a disclosed method of treating a subject can further comprise generating a disclosed AAV viral or non-viral vector. In an aspect, a disclosed method of treating a subject can comprise generating an AAV vector or a recombinant AAV (such as those disclosed herein).

In an aspect, a disclosed method of treating a subject can further comprise gene editing one or more relevant genes (such as, for example, a missing, deficient, and/or mutant protein or enzyme), wherein editing includes but is not limited to single gene knockout, loss of function screening of multiple genes at one, gene knockin, or a combination thereof.

In an aspect of a disclosed method, a payload can comprise one or more base-editing components and one or more sgRNA targeting the region to be edited. In an aspect, a disclosed region to be edited can comprise a W1282X CFTR mutation and/or a R1283G mutation. In an aspect, a disclosed base-editing component can comprise an adenine base editor.

In an aspect, a disclosed method of treating a subject can further comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. In an aspect, a disclosed Cas9 can be from Staphylococcus aureus or Streptococcus pyogenes.

In an aspect, a disclosed method of treating a subject can further comprise generating and/or validating one or more disclosed enzymes or disclosed recombinant enzymes, one or more of the disclosed isolated nucleic acid molecules, one or more disclosed AAV vectors, one or more disclosed pharmaceutical formulations, or any combination thereof.

In an aspect of a disclosed method, a disclosed enzyme, a disclosed recombinant enzyme, a disclosed isolated nucleic acid molecule, a disclosed AAV vector, a disclosed pharmaceutical formulation, or any combination thereof can be delivered and/or administered prior to, concurrent, or after the delivery and/or administration of enzyme replacement therapy, protein replacement, gene therapy, a recombinant product, or any combination thereof.

In an aspect, a disclosed method of treating a subject can further comprise reducing and/or minimizing vector-mediated immunotoxicity and/or transgene immunogenicity (e.g., the ability to induce specific immunity). In an aspect, vector-mediated immunotoxicity and/or transgene immunogenicity can decrease and/or reduce the efficacy of the recombinant product encoded by the transgene. In an aspect, vector-mediated immunotoxicity and/or transgene immunogenicity can decrease and/or reduce the ability and/or likelihood of re-dosing a subject with one or more disclosed enzymes or recombinant enzymes, one or more of the disclosed isolated nucleic acid molecules, one or more disclosed AAV vectors, one or more disclosed pharmaceutical formulations, or any combination thereof. In an aspect, vector-mediated immunotoxicity and/or transgene immunogenicity can decrease and/or reduce the ability and/or likelihood of re-dosing a subject with gene therapy, enzyme replacement therapy, protein replacement, or any combination thereof.

In an aspect, a disclosed method of treating a subject can further comprise administering one or more times one or more disclosed enzymes or recombinant enzymes, one or more of the disclosed isolated nucleic acid molecules, one or more disclosed AAV vectors, one or more disclosed pharmaceutical formulations, or any combination thereof.

In an aspect, a disclosed method of treating a subject can further comprise measuring and/or determining a subject's pre-treatment level of one or more clinical and/or metabolic indicators (such as, for example, the expression of CFTR). In an aspect, a disclosed method can further comprise measuring and/or determining one or more times a subject's level of one or more clinical and/or metabolic indicators (such as CFTR).

### D. Miscellaneous

Disclosed herein is a method of introducing into an organ of a subject a recombinant AAV vector, the method comprising injecting into the organ the recombinant AAV vector such that the AAV vector is introduced into the organ. In an aspect, a disclosed organ can comprise the intestine. In an aspect, a disclosed organ can comprise the colon. In an aspect, a disclosed recombinant AAV vector can be injected into the submucosa of the organ. In an aspect, a disclosed AAV vector can be injected by colonoscopy-guided injection into the submucosa of the organ. In an aspect, a disclosed AAV vector can comprise a cross-species compatible AAV recombinant vector. In an aspect, a disclosed AAV vector can comprise AAV.cc47. In an aspect, a disclosed recombinant AAV vector can comprise an AAV capsid protein variant, wherein the capsid protein variant comprises a peptide having the sequence of SEQ ID NO:58 or a sequence with at least 90% or at least 95% identity thereto. In an aspect, a disclosed recombinant AAV vector can comprise an AAV capsid protein variant, wherein the AAV capsid variant comprises the sequence of SEQ ID NO:58, or a sequence with 1-10, 11-20, 20-30, or 30-50 amino acid substitutions relative thereto. In an aspect, a disclosed recombinant AAV vector can comprise a vector genome. In an aspect, a disclosed recombinant AAV vector genome can be encapsulated by an AAV capsid comprising the AAV capsid protein variant. In an aspect, a disclosed vector genome can comprise a transgene located between the first ITR and the second ITR. In an aspect, a disclosed transgene can encode a molecule selected from the group consisting of a therapeutic RNA, a therapeutic protein, a gene-editing molecule, a nuclease and combinations thereof. In an aspect, a disclosed transgene can encode a nuclease. In an aspect, a disclosed nuclease can comprise a Cas9 nuclease. In an aspect, a disclosed nuclease can comprise a Cas12a nuclease. In an aspect, a disclosed transgene can comprise a gene-editing molecule. In an aspect, a disclosed gene editing molecule can comprise a guide RNA (gRNA). In an aspect, a disclosed gRNA can comprise a single guide RNA (sgRNA). In an aspect, a disclosed AAV capsid protein variant can comprise a peptide having the sequence of SEQ ID NO:58.

TABLE 3
Sequences in Sequence Listing

| SEQ ID NO | SEQUENCE DESCRIPTION |
|---|---|
| 1 | WT Capsid from AAV1 |
| 2 | WT Capsid from AAV2 |
| 3 | WT Capsid from AAV3b |
| 4 | WT Capsid from AAV4 |
| 5 | WT Capsid from AAV5 |
| 6 | WT Capsid from AAV6 |
| 7 | WT Capsid from AAV7 |
| 8 | WT Capsid from AAV8 |
| 9 | WT Capsid from AAV9 |
| 10 | WT Capsid from AAV10 |
| 11 | WT Capsid from AAV11 |
| 12 | WT Capsid from AAV12 |
| 13 | WT Capsid from AAV13 |
| 14 | WT Capsid from AAV rh8 |
| 15 | WT Capsid from AAV rh10 |
| 16 | WT Capsid from AAV rh32.33 |
| 17 | AAV1 (450-459)-Target Region #1 |
| 18 | AAV1 (451-457)-Target Region #2 |
| 19 | AAV2 (449-458)-Target Region #1 |
| 20 | AAV2 (450-456)-Target Region #2 |
| 21 | AAV3b (449-458)-Target Region #1 |
| 22 | AAV3b (450-456)-Target Region #2 |
| 23 | AAV4 (443-452)-Target Region #1 |
| 24 | AAV4 (444-450)-Target Region #2 |
| 25 | AAV5 (442-451)-Target Region #1 |
| 26 | AAV5 (443-449)-Target Region #2 |
| 27 | AAV6 (450-459)-Target Region #1 |
| 28 | AAV6 (451-457)-Target Region #2 |
| 29 | AAV7 (451-460)-Target Region #1 |
| 30 | AAV7 (452-458)-Target Region #2 |
| 31 | AAV8 (452-461)-Target Region #1 |
| 32 | AAV8 (453-459)-Target Region #2 |
| 33 | AAV9 (410-460)-Target Region #1 |
| 34 | AAV9 (452-458)-Target Region #2 |
| 35 | AAV10 (452-461)-Target Region #1 |
| 36 | AAV10 (453-459)-Target Region #2 |
| 37 | AAV1I (442 -451)-Target Region #1 |
| 38 | AAV11 (443 -449)-Target Region #2 |
| 39 | AAV12 (451-460)-Target Region #1 |
| 40 | AAV12 (452-458)-Target Region #2 |
| 41 | AAV13 (448-457)-Target Region #1 |
| 42 | AAV13 (449-455)-Target Region #2 |
| 43 | AAVrh8 (451-460)-Target Region #1 |
| 44 | AAVrh8 (452-458)-Target Region #2 |
| 45 | AAVrh10 (452-461)-Target Region #1 |
| 46 | AAVrh10 (453-459)-Target Region #2 |
| 47 | AAVrh32.33 (442-451)-Target Region #1 |
| 48 | AAVrh32.33 (443-449)-Target Region #2 |
| 49 | AAV.cc41 (IV 452-458 of AAV9) |
| 50 | AAV.cc42 (IV 452-458 of AAV9) |
| 51 | AAV.cc43 (IV 452-458 of AAV9) |
| 52 | AAV.cc44 (IV 452-458 of AAV9) |
| 53 | AAV.cc45 (IV 452-458 of AAV9) |
| 54 | AAV.cc46 (IV 452-458 of AAV9) |
| 55 | AAV.cc47 (IV 452-458 of AAV9) |
| 56 | AAV.cc48 (IV 452-458 of AAV9) |
| 57 | AAV.cc49 (IV 452-458 of AAV9) |
| 58 | AAV.cc41 (full length) |
| 59 | AAV.cc42 (full length) |
| 60 | AAV.cc43 (full length) |
| 61 | AAV.cc44 (full length) |
| 62 | AAV.cc45 (full length) |
| 63 | AAV.cc46 (full length) |
| 64 | AAV.cc47 (full length) |
| 65 | AAV.cc48 (full length) |
| 66 | AAV.cc49 (full length) |
| 67 | AAV.cc81 (VIII 586-592 of AAV9) |
| 68 | AAV.cc82 (VIII 586-592 of AAV9) |
| 69 | AAV.cc83 (VIII 586-592 of AAV9) |
| 70 | AAV.cc84 (VIII 586-592 of AAV9) |
| 71 | AAV.cc85 (VIII 586-592 of AAV9) |
| 72 | AAV.cc86 (VIII 586-592 of AAV9) |
| 73 | AAV.cc87 (VIII 586-592 of AAV9) |
| 74 | AAV.cc88 (VIII 586-592 of AAV9) |
| 75 | AAV.cc89 (VIII 586-592 of AAV9) |
| 76 | AAV.cc81 (full length) |
| 77 | AAV.cc82 (full length) |
| 78 | AAV.cc83 (full length) |
| 79 | AAV.cc84 (full length) |
| 80 | AAV.cc85 (full length) |
| 81 | AAV.cc86 (full length) |
| 82 | AAV.cc87 (full length) |
| 83 | AAV.cc88 (full length) |
| 84 | AAV.cc89 (full length) |
| 85 | AAV.cc41-81 (full length) |
| 86 | AAV.cc41-82 (full length) |
| 87 | AAV.cc41-83 (full length) |
| 88 | AAV.cc41-84 (full length) |
| 89 | AAV.cc41-85 (full length) |
| 90 | AAV.cc41-86 (full length) |
| 91 | AAV.cc41-87 (full length) |
| 92 | AAV.cc41-88 (full length) |
| 93 | AAV.cc41-89 (full length) |
| 94 | AAV.cc42-81 (full length) |
| 95 | AAV.cc42-82 (full length) |
| 96 | AAV.cc42-83 (full length) |
| 97 | AAV.cc42-84 (full length) |
| 98 | AAV.cc42-85 (full length) |
| 99 | AAV.cc42-86 (full length) |
| 100 | AAV.cc42-87 (full length) |
| 101 | AAV.cc42-88 (full length) |
| 102 | AAV.cc42-89 (full length) |
| 103 | AAV.cc43-81 (full length) |
| 104 | AAV.cc43-82 (full length) |
| 105 | AAV.cc43-83 (full length) |
| 106 | AAV.cc43-84 (full length) |
| 107 | AAV.cc43-85 (full length) |
| 108 | AAV.cc43-86 (full length) |
| 109 | AAV.cc43-87 (full length) |
| 110 | AAV.cc43-88 (full length) |
| 111 | AAV.cc43-89 (full length) |
| 112 | AAV.cc44-81 (full length) |
| 113 | AAV.cc44-82 (full length) |
| 114 | AAV.cc44-83 (full length) |
| 115 | AAV.cc44-84 (full length) |
| 116 | AAV.cc44-85 (full length) |
| 117 | AAV.cc44-86 (full length) |
| 118 | AAV.cc44-87 (full length) |
| 119 | AAV.cc44-88 (full length) |
| 120 | AAV.cc44-89 (full length) |
| 121 | AAV.cc45-81 (full length) |
| 122 | AAV.cc45-82 (full length) |
| 123 | AAV.cc45-83 (full length) |
| 124 | AAV.cc45-84 (full length) |
| 125 | AAV.cc45-85 (full length) |
| 126 | AAV.cc45-86 (full length) |
| 127 | AAV.cc45-87 (full length) |
| 128 | AAV.cc45-88 (full length) |
| 129 | AAV.cc45-89 (full length) |
| 130 | AAV.cc46-81 (full length) |
| 131 | AAV.cc46-82 (full length) |
| 132 | AAV.cc46-83 (full length) |
| 133 | AAV.cc46-84 (full length) |
| 134 | AAV.cc46-85 (full length) |
| 135 | AAV.cc46-86 (full length) |
| 136 | AAV.cc46-87 (full length) |
| 137 | AAV.cc46-88 (full length) |
| 138 | AAV.cc46-89 (full length) |
| 139 | AAV.cc47-81 (full length) |
| 140 | AAV.cc47-82 (full length) |
| 141 | AAV.cc47-83 (full length) |
| 142 | AAV.cc47-84 (full length) |
| 143 | AAV.cc47-85 (full length) |
| 144 | AAV.cc47-86 (full length) |
| 145 | AAV.cc47-87 (full length) |
| 146 | AAV.cc47-88 (full length) |
| 147 | AAV.cc47-89 (full length) |
| 148 | AAV.cc48-81 (full length) |
| 149 | AAV.cc48-82 (full length) |
| 150 | AAV.cc48-83 (full length) |
| 151 | AAV.cc48-84 (full length) |
| 152 | AAV.cc48-85 (full length) |
| 153 | AAV.cc48-86 (full length) |
| 154 | AAV.cc48-87 (full length) |
| 155 | AAV.cc48-88 (full length) |
| 156 | AAV.cc48-89 (full length) |
| 157 | AAV.cc49-81 (full length) |
| 158 | AAV.cc49-82 (full length) |
| 159 | AAV.cc49-83 (full length) |
| 160 | AAV.cc49-84 (full length) |
| 161 | AAV.cc49-85 (full length) |
| 162 | AAV.cc49-86 (full length) |
| 163 | AAV.cc49-87 (full length) |
| 164 | AAV.cc49-88 (full length) |
| 165 | AAV.cc49-89 (full length) |
| 166 | WT Capsid from hum.8 |
| 167 | WT Capsid from AAV DJ |
| 168 | WT Capsid from AAV CAM130 |
| 169 | WT Capsid from AAV 1RX |
| 170 | WT Capsid from AAV 218g9 |
| 171 | WT Capsid from AAV 4.69 |
| 172 | Variant Capsid from AAV1 |
| 173 | Variant Capsid from AAV2 |
| 174 | Variant Capsid from AAV3b |
| 175 | Variant Capsid from AAV4 |
| 176 | Variant Capsid from AAV5 |
| 177 | Variant Capsid from AAV6 |
| 178 | Variant Capsid from AAV7 |
| 179 | Variant Capsid from AAV8 |
| 180 | Variant Capsid from AAV9 |
| 181 | Variant Capsid from AAV10 |
| 182 | Variant Capsid from AAV11 |
| 183 | Variant Capsid from AAV12 |
| 184 | Variant Capsid from AAV13 |
| 185 | Variant Capsid from AAVrh8 |
| 186 | Variant Capsid from AAVrh10 |
| 187 | Variant Capsid from AAVrh32.33 |
| 188 | Variant Capsid from AAV1 |
| 189 | Variant Capsid from AAV2 |
| 190 | Variant Capsid from AAV3b |
| 191 | Variant Capsid from AAV4 |
| 192 | Variant Capsid from AAV5 |
| 193 | Variant Capsid from AAV6 |
| 194 | Variant Capsid from AAV7 |
| 195 | Variant Capsid from AAV8 |
| 196 | Variant Capsid from AAV9 |
| 197 | Variant Capsid from AAV10 |
| 198 | Variant Capsid from AAV11 |
| 199 | Variant Capsid from AAV12 |
| 200 | Variant Capsid from AAV13 |
| 201 | Variant Capsid from AAVrh8 |
| 202 | Variant Capsid from AAVrh10 |
| 203 | Variant Capsid from AAVrh32.33 |

### VII. EXAMPLES

Cystic Fibrosis (CF) is a chronic disease affecting multiple organs due to mutations in the CFTR gene. Current therapies have limited efficacy, and gene editing is a promising strategy for permanent CFTR restoration. However, CRISPR/Cas9-mediated gene editing has low efficiency and off-target effects, while base editors (BEs) provide precise changes without double-stranded breaks or off-target effects. In vivo delivery of BEs to target organs is challenging, especially to somatic tissues such as the gastrointestinal tract, which require efficient delivery to stem cells for successful editing.

### Example 1

### Treatment of Cystic Fibrosis with Base Editing Components

Cystic fibrosis (CF) is an inherited disease caused by mutations in the cystic fibrosis transmembrane receptor (CFTR) gene. CF causes mucus accumulation in the lungs and intestines. Many CF mutations do not respond to drug therapies. Thus, gene editing technology is a promising approach to treat CF. Base editors edit single point mutations in the DNA with minimal to no off-target editing. However, base editing has not been applied to genetic diseases of the intestine, such as CF, in vivo. For this project, it was hypothesized that intravenous and/or colonoscopy-guided delivery of base editing components using an adeno-associated virus (AAV) will successfully repair the W1282 CTFR point mutation in the intestine. A novel AAV was developed that infects intestinal stem cells with remarkably high (>70%) efficiency. A base editor was optimized and sgRNA was targeted to successfully repair the W1282X CTFR mutation in vitro. The ability of this optimized AAV-base editor system was then assessed for its ability to repair the W1282X CFTR point mutation in a mouse model and to edit intestinal stem cells in a porcine model. Results from these studies will lead to the development of this technology to target intestinal disease associated with CF and other inborn genetic disorders.

The nonsense W1282X mutation in the cystic fibrosis transmembrane conductance regulator (CFTR) gene leads to the production of a truncated CFTR protein, causing a severe disease form of cystic fibrosis. Current therapies are targeted at restoring function to the dysfunctional CFTR protein, which are not applicable for patients who altogether lack intact the CFTR protein.

Decreased mRNA and protein stability of W1282X limits response to modulator therapy. Gene editing to correct the mutation presents an exciting and novel alternative therapy for these mutations. Base editors are a promising approach for gene-editing because they demonstrate higher efficiency and fewer off-target effects compared to commonly-used CRISPR/Cas9 gene-editing approaches that result in double-stranded DNA breaks.

Adenine base editors (ABEs), consisting of a partially inactivated Cas9 protein fused to an adenosine deaminase, convert A⋅T base pairs to G⋅C when paired with a single guide RNA targeting the base of interest. The long-term goal of this project is to perform in vivo base editing for the W1282X mutation.

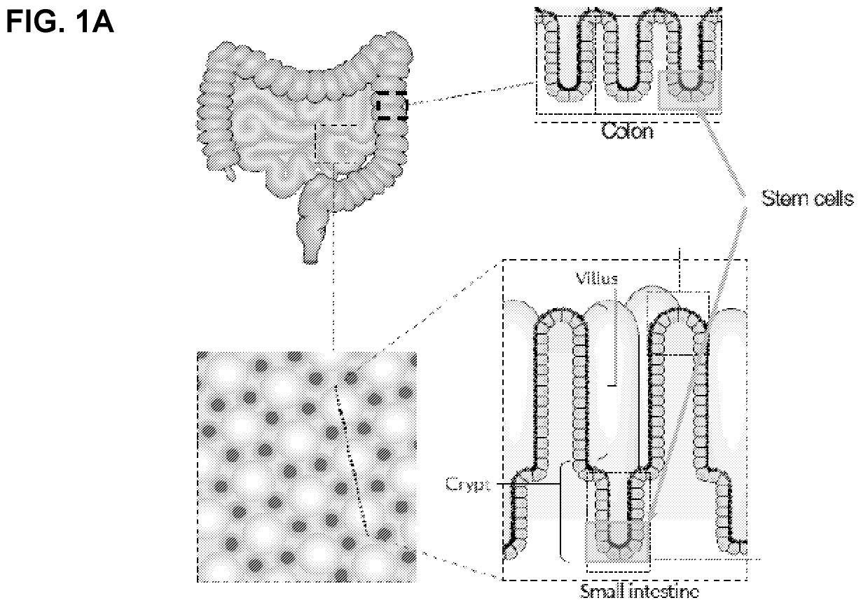

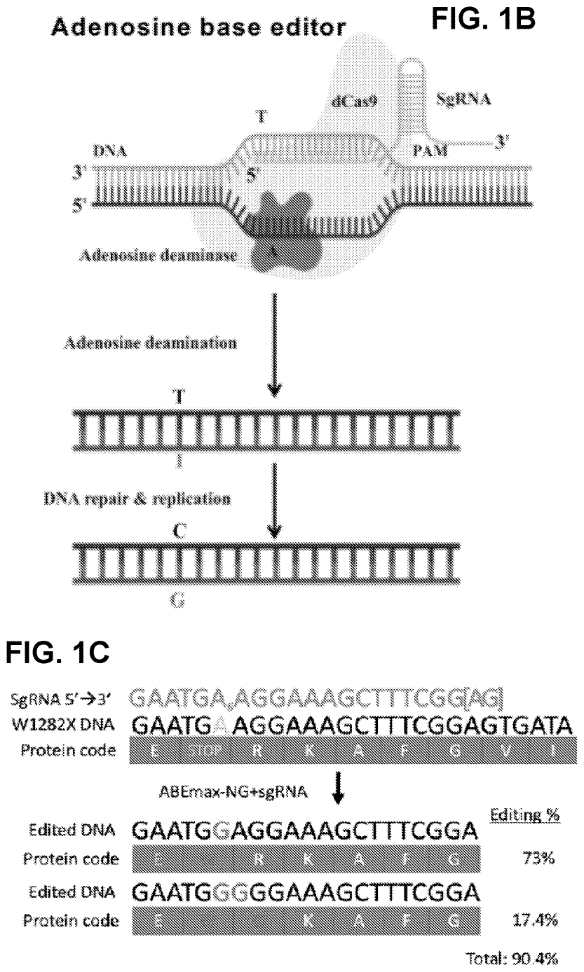

FIG. 1A-FIG. 1C show somatic gene editing in the intestine in vivo by base editing is predicted to repair the W1282X nonsense mutation in accordance with one embodiment of the present disclosure. FIG. 1A shows the intestinal epithelium demonstrating the crypt-villi structure (orange boxes indicate the position of intestinal stem cells) (adopted from Gehart H, et al. (2018) Nat Rev Gastroenterol Hepatol. 16:19-34). FIG. 1B shows the ABE is guided to a specific DNA segment by a guide RNA (SgRNA), with a target base (A₆) within the editing window of the ABE (position 4-7). The ABE results in an A to G conversion (adopted from Yarra R, et al. (2021) Plant Cell Rep. 40:595-604). FIG. 1C shows the proposed BE approach in the W1282X CF mouse model. The protospacer adjacent motif (PAM) of the sgRNA is in brackets. The most common (73%) edit restores the wild-type tryptophan AA. The second most common (17.4%) produces tryptophan and a likely clinically insignificant R1283G mutation. Together, the expected likelihood to restore the CFTR protein to functionality is 90.4% of all edits produced by the BE strategy. BE predictions from BE HIVE (www.crisprbehive.design).

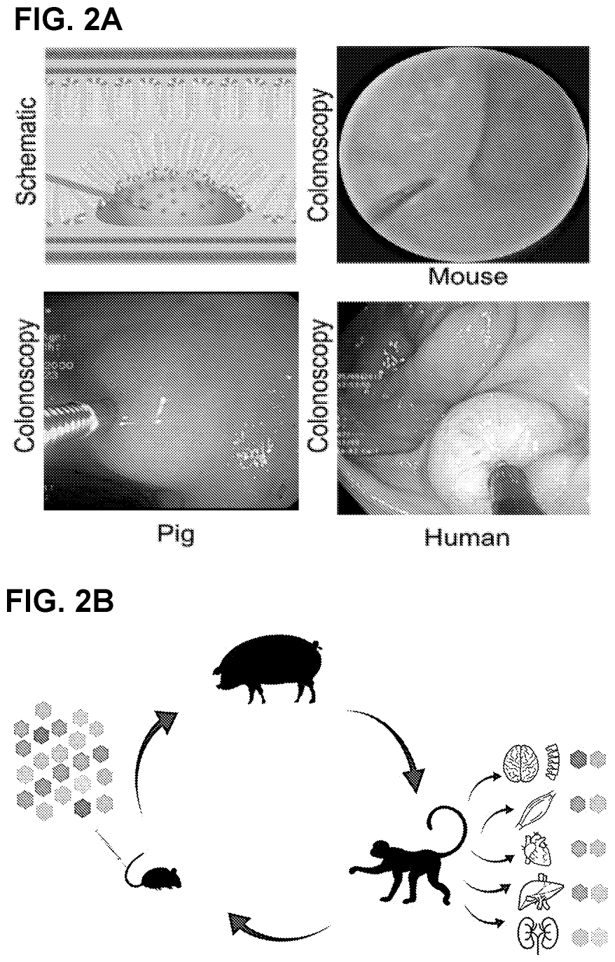

FIG. 2A-FIG. 2B show images and schematics of colonoscopy-guided injection and evolution scheme of cross-species compatible AAVs (ccAAVs) in accordance with one embodiment of the present disclosure. FIG. 2A is a schematic of colonoscopy-guided injection demonstrating placement of the needle into the submucosa, resulting in a “bubble” under the mucosa, as well as representative colonoscopic images from mouse, pig, and human, demonstrating the cross-species utility of the experimental approach. FIG. 2B shows capsid libraries are cycled through mice, pigs, and monkeys. The parental capsid library is injected in mice and AAV genomes are amplified from murine tissues, generating a new capsid library, which is then injected into a pig. This process is repeated until a final capsid library is generated from the rhesus macaque tissue harvest. This process generates AAVs with the highest tropism for target tissues in multiple species and greatest potential for clinical translation into humans.

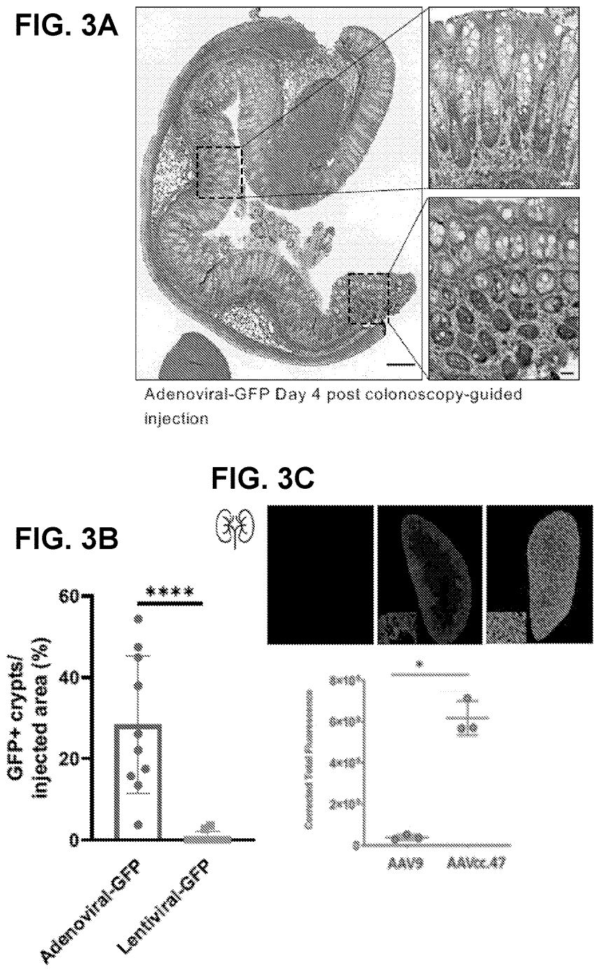

FIG. 3A-FIG. 3C shows data showing that colonoscopy-guided injection of adenoviral GFP efficiently transduces colonic stem cells and AAV.cc47 transduces target tissues with high efficiency compared to AAV9 and AAV9 and AAV.cc47 packaging self-complementary Cbh-mCherry were delivered IV (1e12 vg/mouse) in accordance with one embodiment of the present disclosure. FIG. 3A shows a representative image of immunohistochemistry labeling of GFP+ cells (brown staining) in murine colonic tissue collected 4 days after colonoscopy-guided injection of adenoviral-GFP. Scale bar is 200 μm. Insets demonstrate GFP+ colonic epithelium. Scale bar is 20 m. FIG. 3B shows the number of colonic crypts with >2 GFP+ cells were quantified as a percentage of the injected area for adenoviral-GFP and lentiviral-GFP (identified as the cross section with GFP positivity in the muscularis mucosae). n=10 animals; ****p<0.0001. FIG. 3C shows representative images and total mCherry Fluorescence in the kidney 28 days post injection are shown (insets=white dotted line). *p<0.05.

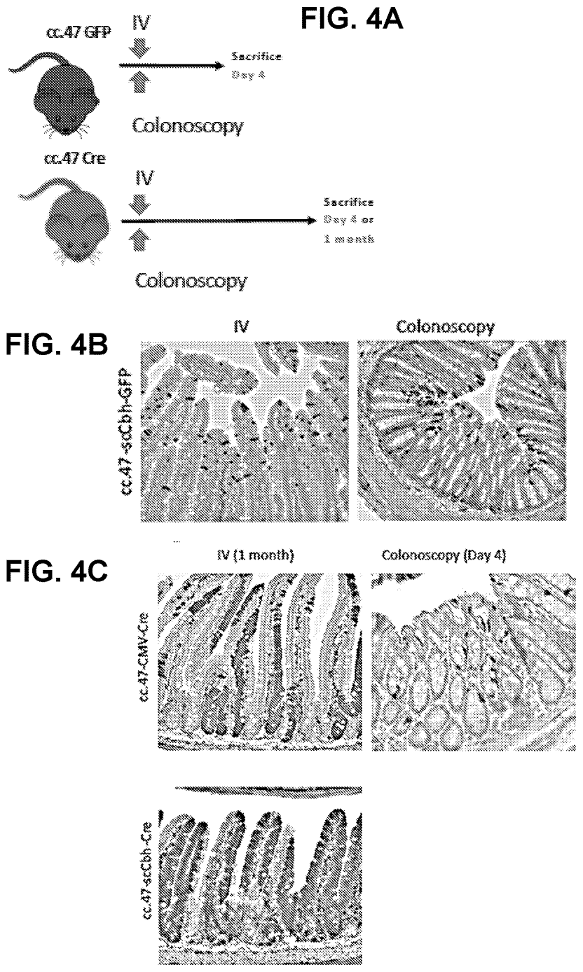

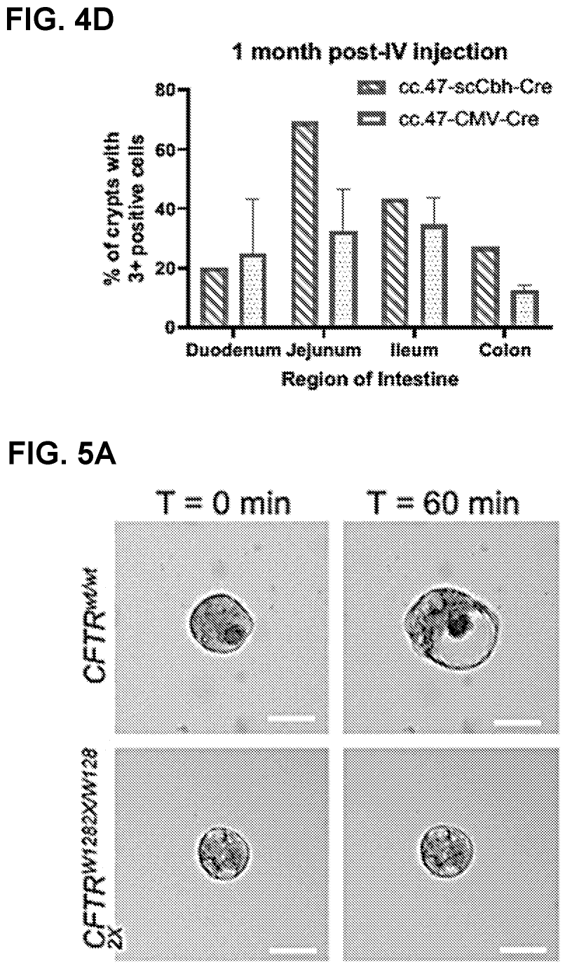

FIG. 4A-FIG. 4D provide images, graphs, and schematics showing AAVcc.47 results in high efficiency of intestinal epithelial transfection. FIG. 4A shows schematic of evaluation of cc.47 carrying either a fluorescent marker, GFP or Cre (for injection into R26R-LSL-tdTomato mice). Mice were injected by intravenous or colonoscopy-guided routes, then sacrificed at various timepoints to evaluate efficiency of viral transfection. FIG. 4B provides representative IHC images of intestine collected 4 days after injection of AAVcc.47-scCbh-GFP by IV or colonoscopy routes (n=10 per group). Brown-stained cells have expressed the GFP introduced by the virus. FIG. 4C shows representative IHC images of intestine collected 4 days or one month after injection of either AAVcc.47-CMV-Cre or AAVcc.47-scCbh-Cre by IV or colonoscopy routes. Brown-stained cells have expressed tdTomato after removal of the STOP codon by Cre expression. FIG. 4D shows preliminary quantification of the % of lineage trace events (indicating stem cell transfection) in the indicated groups. N=1 (IV, AAVcc.47-scCbh-Cre), n=3 (IV, AAVcc.47-CMV-Cre).

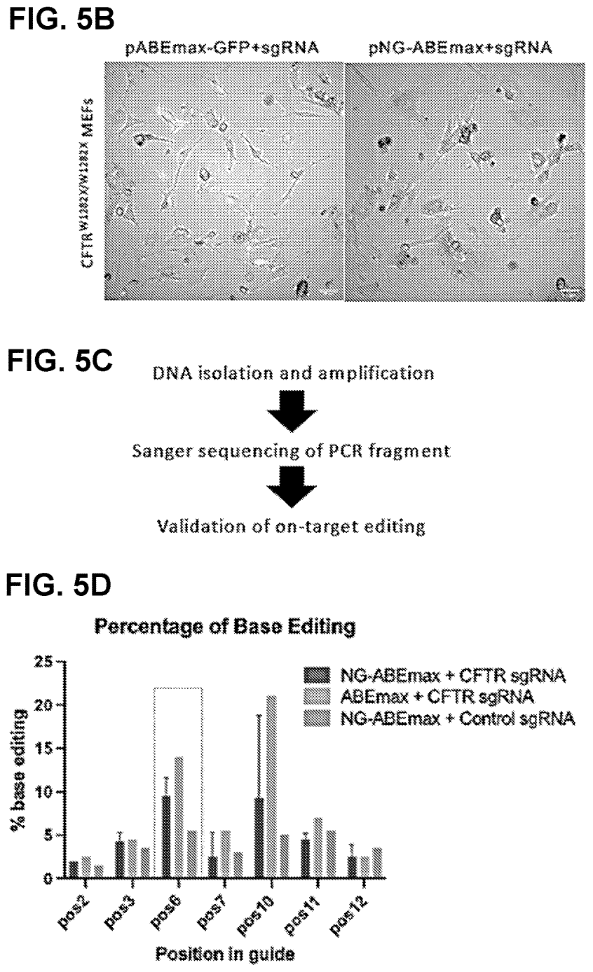

FIG. 5A-FIG. 5D are images, schematics and data showing successful electroporation of CFTR^{W1282X/W1282X} mouse embryonic fibroblasts with ABE and sgRNA to edit W1282X mutation. FIG. 5A shows forskolin stimulation assay was performed on organoids isolated from CFTR^{wt/wt} and CFTR^{W1282X/W1282X} littermates. Organoids were imaged pre and post application of 5 mM forskolin. Scale bar is 20 m. FIG. 5B shows representative images of CFTR^{W1282X/W1282X} mouse embryonic fibroblasts post electroporation with either pABEmax-GFP or pNG-ABEmax and sgRNA targeting the W1282X CFTR mutation. Scale bar is 50 m. Electroporation efficiency based on ABEmax-GFP (% of GFP+ cells/total cells) averaged 24% (n=5). FIG. 5C shows steps for validation of on-target editing using the electroporated MEFs in FIG. 5A. FIG. 5D shows the percentage of base editing in MEFs. After the Sanger sequencing was performed, the sequences were analyzed using EditR software. Percentage of edits was quantified at each adenosine base within the guide sequence as these are the potential sites of base editing with an ABE. The mutated base, and therefore the position of interest, is located at position 6 (denoted by the dashed box). For NG-ABEmax+CFTR sgRNA, n=2. For the other two conditions, n=1. Samples had a transfection efficiency of approximately 25%.

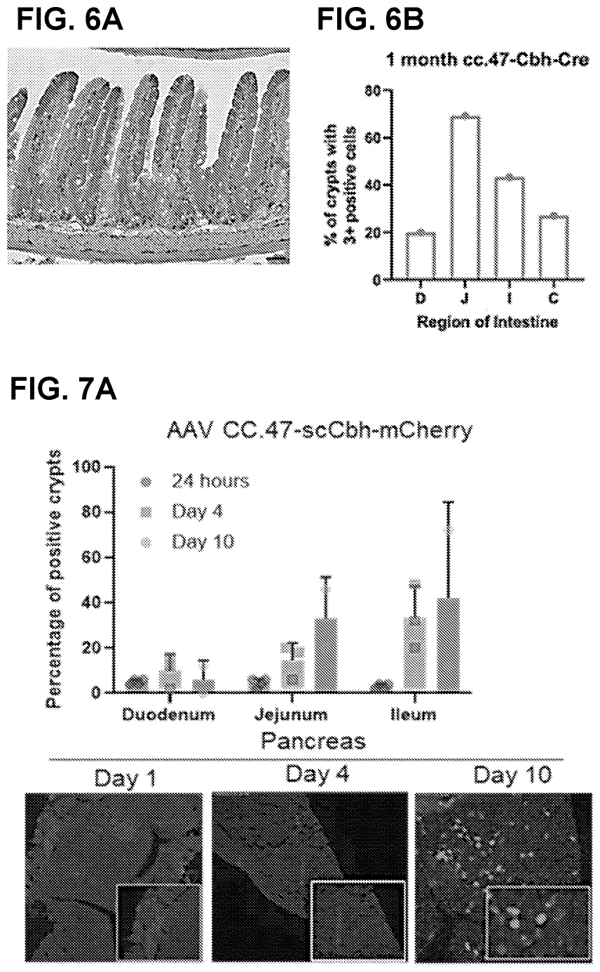

FIG. 6A-FIG. 6B show that AAV.cc47 efficiently transduced intestinal and colonic epithelium. Cbh-Cre packaged by AAV.cc47 were delivered at a dose of 6e11 vg/mouse into LSL-tdTomato reporter mice. FIG. 6A shows tdTomato immunohistochemistry in jejunum. FIG. 6B shows fluorescence in the (D) duodenum, (J) jejunum, (I) ileum, and (C) colon epithelium, which was quantified at one month. Scale bar 20 mm. Evidence for efficient introduction of a recombinant AAV vector into intestine was generated. (FIG. 6A-FIG. 6B)

### Example 2

### Transduction of the Intestinal Epithelium by AAVcc.47

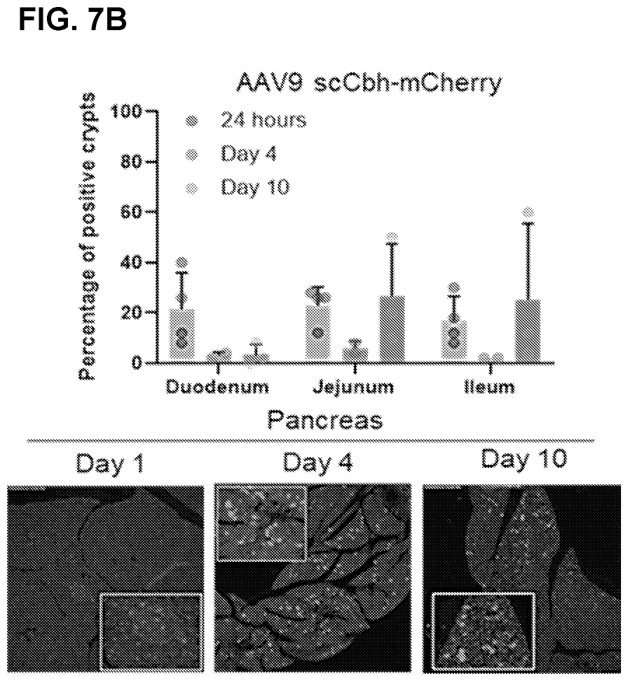

AAVcc.47 and AAV9 carrying an mCherry fluorescent reporter were delivered to wild-type mice, and then the intestine and the pancreas were harvested for analysis of mCherry transduction. The data show that AAVcc.47 transduced both the intestine and the pancreas more efficiently than AAV9 (FIG. 7A-FIG. 7B).

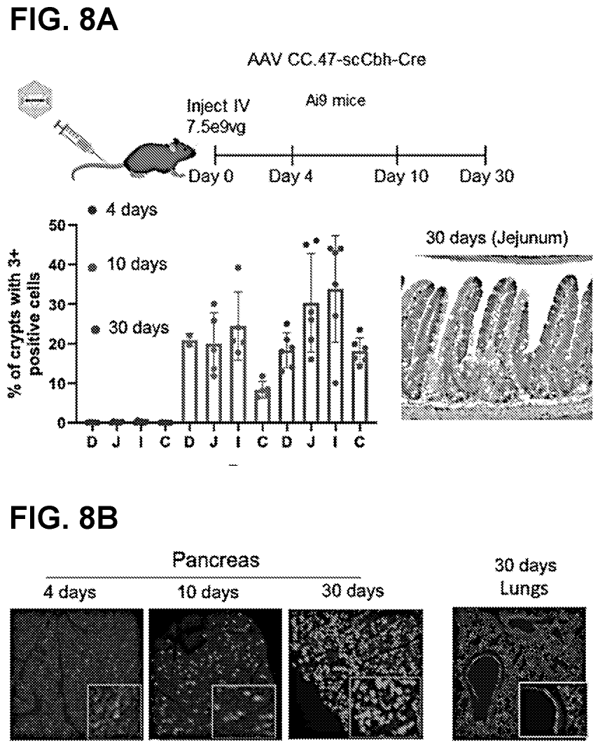

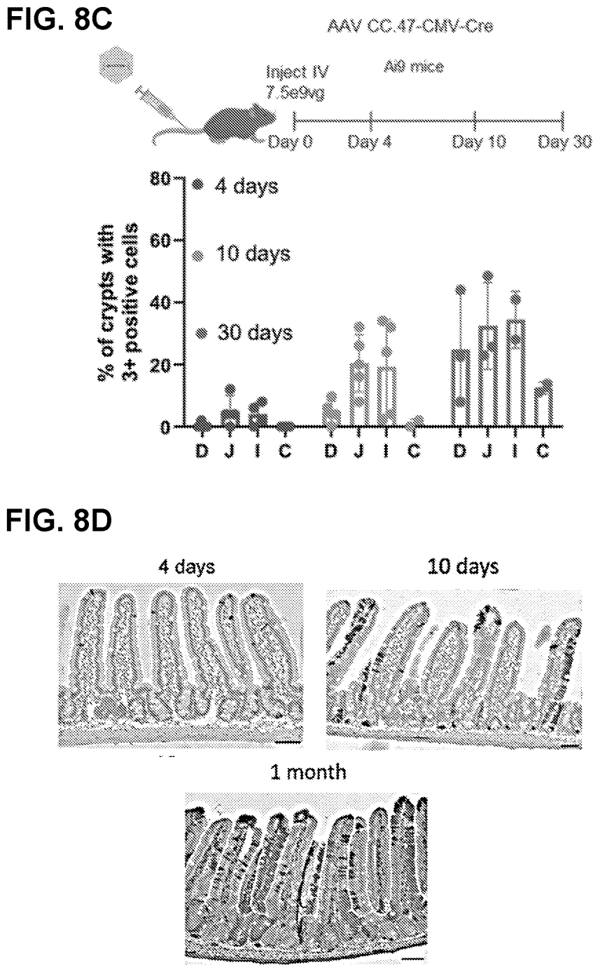

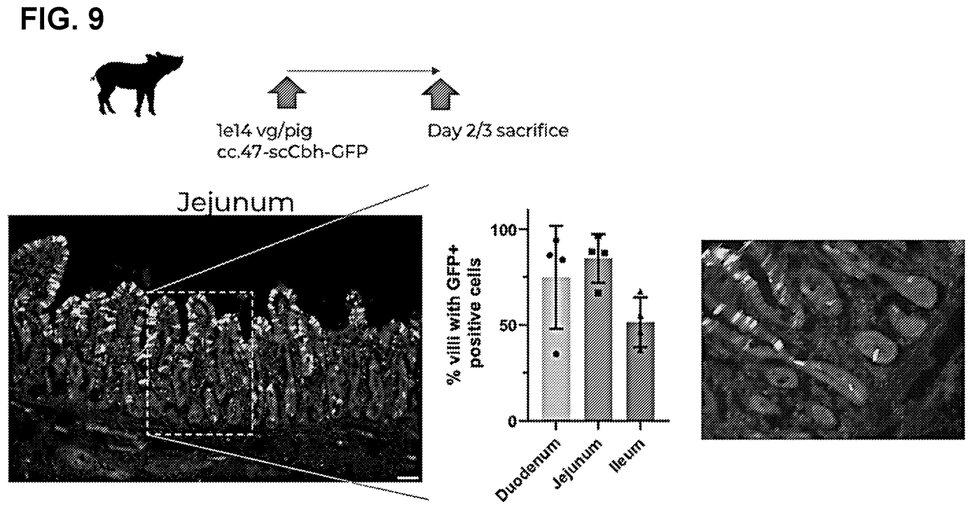

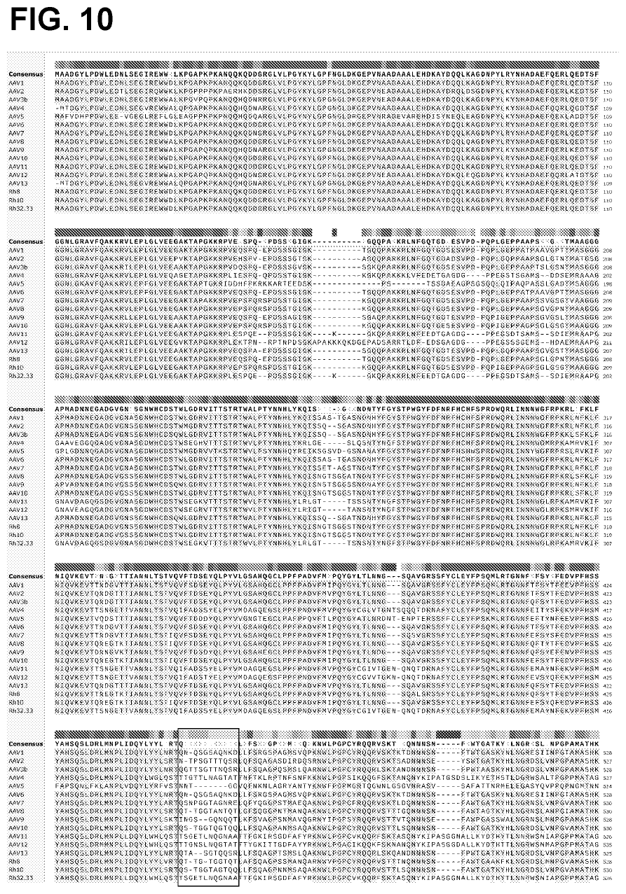

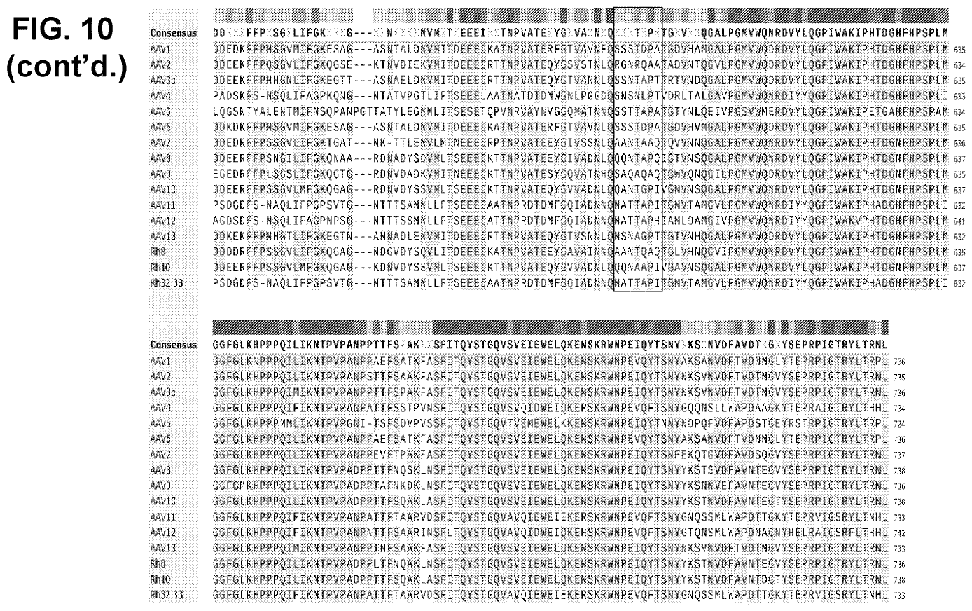

Two versions of AAVcc.47 (i.e., one having a hybrid chicken beta-actin promoter (scCBh-GFP) promoter and one having a CMV promoter) with Cre recombinase were delivered to tdTomato reporter mice (Ai9 mice). Then, the transduction efficiency in the intestinal epithelium, pancreas, and lung were characterized. Transduction was more efficient in the intestinal epithelium using scCbh virus compared to CMV virus. Widespread recombination was also identified in the pancreas and in the lung (FIG. 8A-FIG. 8D). The ability of AAVcc.47 to transduce the intestinal epithelium was then evaluated in a pig model. These studies showed that AAVcc.47 GFP virus transduced intestinal epithelial cells, including stem cells, in a large animal pig model (FIG. 9A-FIG. 9D).

### Example 3

### Transduction of the Intestinal Epithelium by Other AAV Variants

Several disclosed AAV9 variants are used here including those listed in the table below.

| AAV | Identifier | Variant Sequence |
|---|---|---|
| AAV9 (WT) | SEQ ID NO: 09 | NGSGQNQ (WT) |
| AAV.cc41 | SEQ ID NO: 58 | EGGTVHA |
| AAV.cc42 | SEQ ID NO: 59 | FYGTDSA |
| AAV.cc43 | SEQ ID NO: 60 | HGQSASR |
| AAV.cc44 | SEQ ID NO: 61 | DTPTNQA |
| AAV.cc45 | SEQ ID NO: 62 | ITRQAYQ |
| AAV.cc46 | SEQ ID NO: 63 | RMFKSNQ |
| AAV.cc48 | SEQ ID NO: 65 | KHFLQGE |
| AAV.cc49 | SEQ ID NO: 66 | MGRERAG |

Each variant AAV having one or more substitutions in VR4 carries a mCherry fluorescent reporter are selected (along with wild-type AAV) and are delivered to wild-type mice. The intestine and the pancreas are harvested for analysis of mCherry transduction. The data focus on the transduction of both the intestine and the pancreas by each of the disclosed variant AAVs. The level of transduction achieved by each variant AAV is compared to the other variants as well as to that of wild-type AAV9.

Next, two versions of each variant AAV (i.e., one carrying a scChb promoter and one carrying a CMV promoter) with Cre recombinase are delivered to tdTomato reporter mice (i.e., Ai9 mice). Then, the transduction efficiency is characterized for the intestinal epithelium, pancreas, and lung. The transduction is compared between the two versions of each variant AAV as well as to the two versions of wild-type AAV9 and the versions of other variant AAVs.

The ability of each variant AAV to transduce the intestinal epithelium is further evaluated in a pig model. The transduction of transduced intestinal epithelial cells, including stem cells, in a large animal pig model are quantified. Moreover, whether there is a restoration (complete or partial) one or more aspects of cellular homeostasis and/or cellular functionality and/or metabolic dysregulation is determined. Any restoration can be quantified and compared to a pre-existing level such as, for example, a pre-treatment level, or compared to a control level or a reference level (e.g., determined, for example, using one or more subjects not having a missing, deficient, and/or mutant protein or enzyme).

### Example 4

### Transduction of the Intestinal Epithelium by Other AAV Variants

Several disclosed capsid variants from various AAV serotypes are used here including those listed in the table below.

| Serotype | Identifier |
|---|---|
| AAV1 | SEQ ID NO: 172 or SEQ ID NO: 188 |
| AAV2 | SEQ ID NO: 173 or SEQ ID NO: 189 |
| AAV3 | SEQ ID NO: 174 or SEQ ID NO: 190 |
| AAV4 | SEQ ID NO: 175 or SEQ ID NO: 191 |
| AAV5 | SEQ ID NO: 176 or SEQ ID NO: 192 |
| AAV6 | SEQ ID NO: 177 or SEQ ID NO: 196 |
| AAV7 | SEQ ID NO: 178 or SEQ ID NO: 194 |
| AAV8 | SEQ ID NO: 179 or SEQ ID NO: 195 |
| AAV9 | SEQ ID NO: 180 or SEQ ID NO: 196 |
| AAV10 | SEQ ID NO: 181 or SEQ ID NO: 197 |
| AAV11 | SEQ ID NO: 182 or SEQ ID NO: 198 |
| AAV12 | SEQ ID NO: 183 or SEQ ID NO: 199 |
| AAV13 | SEQ ID NO: 184 or SEQ ID NO: 200 |
| AAV rh8 | SEQ ID NO: 185 or SEQ ID NO: 201 |
| AAV rh10 | SEQ ID NO: 186 or SEQ ID NO: 202 |
| AAV rh32.33 | SEQ ID NO: 187 or SEQ ID NO: 203 |

Each variant AAV having one or more substitutions in VR4 carries a mCherry fluorescent reporter are selected (along with wild-type AAV) and are delivered to wild-type mice. The intestine and the pancreas are harvested for analysis of mCherry transduction. The data focus on the transduction of both the intestine and the pancreas by each of the disclosed variant AAVs. The level of transduction achieved by each variant AAV is compared to the other variants as well as to that of wild-type AAV9.

Next, two versions of each variant AAV (i.e., one carrying a scChb promoter and one carrying a CMV promoter) with Cre recombinase are delivered to tdTomato reporter mice (Ai9 mice). Then, the transduction efficiency is characterized for the intestinal epithelium, pancreas, and lung. The transduction is compared between the two versions of each variant AAV as well as to the two versions of wild-type AAV9 and the versions of other variant AAVs. The ability of each variant AAV to transduce the intestinal epithelium is further evaluated in a pig model. The transduction of transduced intestinal epithelial cells, including stem cells, in a large animal pig model is quantified.

Moreover, whether there is a restoration (complete or partial) one or more aspects of cellular homeostasis and/or cellular functionality and/or metabolic dysregulation is determined. Any restoration can be quantified and compared to a pre-existing level such as, for example, a pre-treatment level, or compared to a control level or a reference level (e.g., determined, for example, using one or more subjects not having a missing, deficient, and/or mutant protein or enzyme).

### Example 5

### Transduction of the Intestinal Epithelium by Other AAV Variants Having Substitutions in VR8

Several disclosed variant AAVs (each having one or more substitutions in Variable Region 8 (VIII)) are used here including those listed in the table below.

| AAV | Identifier | Variant Sequence |
|---|---|---|
| AAV9 (WT) | SEQ ID NO: 09 | SAQAQAQ (WT) |
| AAV.cc81 | SEQ ID NO: 76 | LNSSVPS |
| AAV.cc82 | SEQ ID NO: 77 | YMDHQVS |
| AAV.cc83 | SEQ ID NO: 78 | TSDSLVS |
| AAV.cc84 | SEQ ID NO: 79 | NAVGALS |
| AAV.cc85 | SEQ ID NO: 80 | MPISHHE |
| AAV.cc86 | SEQ ID NO: 81 | DSGARGA |
| AAV.cc87 | SEQ ID NO: 82 | NVALALG |
| AAV.cc88 | SEQ ID NO: 83 | GALRMGM |
| AAV.cc89 | SEQ ID NO: 84 | LSGEGAV |

Each variant AAV having one or more substitutions in VR8 carries a mCherry fluorescent reporter are selected (along with wild-type AAV) and are delivered to wild-type mice. The intestine and the pancreas are harvested for analysis of mCherry transduction. The data focus on the transduction of both the intestine and the pancreas by each of the disclosed variant AAVs. The level of transduction achieved by each variant AAV is compared to the other variants as well as to that of wild-type AAV9.

Next, two versions of each variant AAV (i.e., one carrying a scChb promoter and one carrying a CMV promoter) with Cre recombinase are delivered to tdTomato reporter mice (i.e., Ai9 mice). Then, the transduction efficiency is characterized for the intestinal epithelium, pancreas, and lung. The transduction is compared between the two versions of each variant AAV as well as to the two versions of wild-type AAV9 and the versions of other variant AAVs.

The ability of each variant AAV to transduce the intestinal epithelium is further evaluated in a pig model. The transduction of transduced intestinal epithelial cells, including stem cells, in a large animal pig model are quantified. Moreover, whether there is a restoration (complete or partial) one or more aspects of cellular homeostasis and/or cellular functionality and/or metabolic dysregulation is determined. Any restoration can be quantified and compared to a pre-existing level such as, for example, a pre-treatment level, or compared to a control level or a reference level (e.g., determined, for example, using one or more subjects not having a missing, deficient, and/or mutant protein or enzyme).

### Example 6

### Transduction of the Intestinal Epithelium by Other AAV Variants Having Substitutions in VR4 and VR8

Several disclosed variant AAVs (each having one or more substitutions in Variable Region 8 (VIII)) are used here including those provided, for example, in (i) any one of SEQ ID NO:85-SEQ ID NO:93, (ii) any one of SEQ ID NO:94-SEQ ID NO: 102, (iii) any one of SEQ ID NO:103-SEQ ID NO:111, (iv) any one of SEQ ID NO:112-SEQ ID NO:120, (v) any one of SEQ ID NO:121-SEQ ID NO: 129, (vi) any one of SEQ ID NO:130-SEQ ID NO:138, (vii) any one SEQ ID NO: 139-SEQ ID NO: 147, (viii) any one of SEQ ID NO: 148-SEQ ID NO: 156, and (viii) any one of SEQ ID NO: 157-SEQ ID NO: 165.

Each variant AAV having one or more substitutions in VR4 and VR8 carries a mCherry fluorescent reporter are selected (along with wild-type AAV) and are delivered to wild-type mice. The intestine and the pancreas are harvested for analysis of mCherry transduction. The data focus on the transduction of both the intestine and the pancreas by each of the disclosed variant AAVs. The level of transduction achieved by each variant AAV is compared to the other variants as well as to that of wild-type AAV9.

Next, two versions of each variant AAV (i.e., one carrying a scChb promoter and one carrying a CMV promoter) with Cre recombinase are delivered to tdTomato reporter mice (i.e., Ai9 mice). Then, the transduction efficiency is characterized for the intestinal epithelium, pancreas, and lung. The transduction is compared between the two versions of each variant AAV as well as to the two versions of wild-type AAV9 and the versions of other variant AAVs.

The ability of each variant AAV to transduce the intestinal epithelium is further evaluated in a pig model. The transduction of transduced intestinal epithelial cells, including stem cells, in a large animal pig model are quantified. Moreover, whether there is a restoration (complete or partial) one or more aspects of cellular homeostasis and/or cellular functionality and/or metabolic dysregulation is determined. Any restoration can be quantified and compared to a pre-existing level such as, for example, a pre-treatment level, or compared to a control level or a reference level (e.g., determined, for example, using one or more subjects not having a missing, deficient, and/or mutant protein or enzyme).

### SUMMARY OF EXAMPLES

Together, these studies established that AAV vectors having variant capsid proteins have an improved tropism for intestinal epithelium and/or colonic epithelium. This finding holds significant and broad applications for gene therapy for a wide range of gastrointestinal disorders.

What is claimed is:

1. A variant AAV capsid protein comprising the sequence set forth in any one of SEQ ID NO:172-SEQ ID NO:203.

2. A variant AAV capsid protein comprising the sequence set forth in any one of SEQ ID NO:58-SEQ ID NO:66.

3. An isolated nucleic acid molecule comprising a nucleic acid sequence encoding the variant AAV capsid protein of claim 1 or claim 2.

4. An isolated nucleic acid molecule comprising a nucleic acid sequence encoding the variant AAV capsid protein of claim 1 or claim 2 and a nucleic acid sequence encoding a transgene and/or a payload.

5. An AAV vector comprising a nucleic acid sequence encoding the variant AAV capsid protein of claim 1 or claim 2.

6. An AAV vector comprising a nucleic acid sequence encoding the variant AAV capsid protein of claim 1 or claim 2 and a nucleic acid sequence encoding a transgene and/or a payload.

7. The AAV vector of claim 6, wherein the transgene and/or payload encodes one or more base editing components.

8. The AAV vector of claim 7, wherein the one or more base editing components comprise an adenine base editor.

9. The AAV vector of claim 7 or claim 8, further comprising a nucleic acid sequence encoding one or more gRNAs.

10. The AAV vector of claim 9, wherein the one or more gRNAs target the CFTR gene.

11. The AAV vector of claim 10, wherein the one or more gRNAs target the W1282X nonsense mutation.

12. The AAV vector of any one of claim 5-9, wherein the vector demonstrates an improved tropism for intestinal epithelium and/or colonic epithelium when compared to a wild-type AAV vector.

13. A cell comprising the AAV vector of any one of claims 5-claim 12.

14. A pharmaceutical formulation, comprising: the AAV vector of any one of claims 5-12.

15. A method of treating a subject, the method comprising:
administering to a subject in need thereof the AAV vector of any one of claims 5-12 or the pharmaceutical formulation of claim 14;
wherein, following the administering of the vector or the pharmaceutical formulation, one or more aspects of cellular homeostasis and/or cellular functionality in the subject is improved and/or restored.

16. The method of claim 15, wherein the subject has one or more genetic diseases or disorders that affect the intestines and/or colon.

17. The method of claim 16, wherein the subject has cystic fibrosis.

18. The method of any one of claims 15-18, wherein administering comprises intravenous administration and/or colonoscopy guided administration to the intestines and/or colon.

19. The method of any one of claims 15-18, wherein the improved and/or restored cellular homeostasis and/or cellular functionality occurs in the intestinal epithelium and/or the colonic epithelium.

20. The method of any one of claims 15-19, wherein the expression of CFTR is increased and/or enhanced when compared to a pre-treatment expression level.

21. The method of any one of claims 15-20, further comprising administering one or more additional therapeutic agents.

22. The method of claim 21, wherein the one or more additional therapeutic agents comprise pancreatic enzyme replacement therapy (PERT).

23. The method of any one of claims 15-22, wherein following the administering step, the subject's risk of developing a pulmonary infection and/or progressive pulmonary infection is decreased.
